(19) United States
(12) Patent Application Publication
(10) Pub. No.: US 2024/0247266 A1
(43) Pub. Date: Jul. 25, 2024

(54) METHODS AND COMPOSITIONS FOR TREATING AN ANGIOTENSINOGEN- (AGT-) ASSOCIATED DISORDER

(71) Applicant: Alnylam Pharmaceuticals, Inc., Cambridge, MA (US)

(72) Inventors: Stephen Albert Huang, Lexington, MA (US); Kenji P. Fujita, Millburn, NJ (US); Kang Lin, Arlington, MA (US); Nitin Mehrotra, Acton, MA (US); Gabriel Robbie, Brookline, MA (US); Jongtae Lee, Wayland, MA (US); Sagar Agarwal, Lexington, MA (US)

(21) Appl. No.: 18/542,950

(22) Filed: Dec. 18, 2023

Related U.S. Application Data
(63) Continuation of application No. PCT/US2022/035523, filed on Jun. 29, 2022.
(60) Provisional application No. 63/276,808, filed on Nov. 8, 2021.
(60) Provisional application No. 63/216,758, filed on Jun. 30, 2021.

(51) Int. Cl. C12N 15/113; A61K 31/4184; A61K 31/713; A61P 9/12

(52) U.S. Cl. CPC C12N 15/1136; A61K 31/4184; A61K 31/713; A61P 9/12; C12N 2310/14; C12N 2310/315; C12N 2310/321; C12N 2310/323; C12N 2310/33; C12N 2310/351

(57) ABSTRACT

The present invention relates to methods of inhibiting the expression of an AGT gene in a subject, as well as methods for treating subjects having an AGT-associated disorder, e.g., hypertension, using RNAi agents, e.g., double stranded RNAi agents, targeting the AGT gene. The invention also relates to methods of decreasing blood pressure levels in a subject using such RNAi agents to inhibit expression of an AGT gene.

### RELATED APPLICATIONS

This application is a 35 § U.S.C. 111(a) continuation application which claims the benefit of priority to PCT/US2022/035523, filed on Jun. 29, 2022, which, in turn, claims the benefit of priority to U.S. Provisional Application No. 63/216,758, filed on Jun. 30, 2021, and U.S. Provisional Application No. 63/276,808, filed on Nov. 8, 2021. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Mar. 13, 2024, is named “121301-16703_SL.xml” and is 53,084 bytes in size.

### BACKGROUND OF THE INVENTION

The renin-angiotensin-aldosterone system (RAAS) plays a crucial role in the regulation of blood pressure. The RAAS cascade begins with the release of renin by the juxtaglomerular cells of the kidney into the circulation. Renin secretion is stimulated by several factors, including Na+ load in the distal tubule, β-sympathetic stimulation, or reduced renal perfusion. Active renin in the plasma cleaves angiotensinogen (produced by the liver) to angiotensin I, which is then converted by circulating and locally expressed angiotensin-converting enzyme (ACE) to angiotensin II. Most of the effects of angiotensin II on the RAAS are exerted by its binding to angiotensin II type 1 receptors (AT₁R), leading to arterial vasoconstriction, tubular and glomerular effects, such as enhanced Na+ reabsorption or modulation of glomerular filtration rate. In addition, together with other stimuli such as adrenocorticotropin, anti-diuretic hormone, catecholamines, endothelin, serotonin, and levels of Mg2+ and K+, AT₁R stimulation leads to aldosterone release which, in turn, promotes Na+ and K+ excretion in the renal distal convoluted tubule.

Dysregulation of the RAAS leading to, for example, excessive angiotensin II production or AT₁R stimulation results in hypertension which can lead to, e.g., increased oxidative stress, promotion of inflammation, hypertrophy, and fibrosis in the heart, kidneys, and arteries, and result in, e.g., left ventricular fibrosis, arterial remodeling, and glomerulosclerosis.

Hypertension is the most prevalent, controllable disease in developed countries, affecting 20-50% of adult populations. Hypertension is a major risk factor for various diseases, disorders and conditions such as, shortened life expectancy, chronic kidney disease, stroke, myocardial infarction, heart failure, aneurysms (e.g. aortic aneurysm), peripheral artery disease, heart damage (e.g., heart enlargement or hypertrophy) and other cardiovascular related diseases, disorders, or conditions. In addition, hypertension has been shown to be an important risk factor for cardiovascular morbidity and mortality accounting for, or contributing to, 62% of all strokes and 49% of all cases of heart disease. In 2017, changes in the guidelines for diagnosis, prevention, and treatment of hypertension were developed providing goals for even lower blood pressure to further decrease risk of development of diseases and disorders associated with hypertension (see, e.g., Reboussin et al. Systematic Review for the 2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. JAm Coll Cardiol. 2017 Nov. 7. pii: S0735-1097(17)41517-8. doi: 10.1016/j.jacc.2017.11.004; and Whelton et al. (2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. JAm Coll Cardiol. 2017 Nov. 7. pii: S0735-1097(17)41519-1. doi: 10.1016/j.jacc.2017.11.006).

Despite the number of anti-hypertensive drugs available for treating hypertension, more than two-thirds of subjects are not controlled with one anti-hypertensive agent and require two or more anti-hypertensive agents selected from different drug classes. This further reduces the number of subjects with controlled blood pressure as adherence is reduced and side-effects are increased with increasing numbers of medications. Furthermore, several studies have suggested a potential relationship between chronic use of antihypertensive medications and deterioration in kidney function finding that antihypertensive agents to control blood pressure also impact kidney function independently of their effect on blood pressure (Tomlinson,et al (2013) PLoS ONE 8(11) Article ID e78465; The SPRINT Research Group (2015) NEJM 373(22):2103-2116, ClinicalTrials.gov number, NCT01206062; Kidney Disease: Improving Global Outcomes (KDIGO) CKD Work Group (2013) Kidney International Supplements 3:1-150; Kamaroff, et al. (2018(Hindawi International J Chron Dis Article ID 1382705).

Accordingly, there is a need in the art for additional methods and therapies to treat subjects having hypertension.

### SUMMARY OF THE INVENTION

The invention provides methods and compositions for inhibiting the expression of an angiotensinogen (AGT) gene, for treating a subject having a disorder that would benefit from reduction in AGT expression, for treating a subject having an AGT-associated disorder, and for decreasing blood pressure in a subject. The methods include administering to the subject a fixed dose of an RNAi agent, e.g., a double stranded RNAi agent, targeting an AGT gene.

In one aspect, the present invention provides a method for inhibiting the expression of an angiotensinogen (AGT) gene in a subject. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a nucleotide sequence comprising at least 19 contiguous nucleotides of the nucleotide sequence UGUACUCUCAUUGUGGAUGACGA (SEQ ID NO: 9) and the sense strand comprises a nucleotide sequence comprising at least 19 contiguous nucleotides of the nucleotide sequence GUCAUCCACAAUGAGAGUACA (SEQ ID NO: 10); wherein the double-stranded RNAi agent, or salt thereof, comprises at least one modified nucleotide; and wherein at least one of the modifications on the nucleotides is a thermally destabilizing nucleotide modification, thereby inhibiting the expression of the AGT gene in the subject.

In another aspect, the present invention provides a method for treating a subject that would benefit from reduction in angiotensinogen (AGT) expression, e.g., a subject at risk of developing an AGT-associated disorder, e.g., hypertension. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a nucleotide sequence comprising at least 19 contiguous nucleotides of the nucleotide sequence UGUACUCUCAUUGUGGAUGACGA (SEQ ID NO: 9) and the sense strand comprises a nucleotide sequence comprising at least 19 contiguous nucleotides of the nucleotide sequence GUCAUCCACAAUGAGAGUACA (SEQ ID NO: 10); wherein the double-stranded RNAi agent, or salt thereof, comprises at least one modified nucleotide; and wherein at least one of the modifications on the nucleotides is a thermally destabilizing nucleotide modification, thereby treating the subject that would benefit from reduction in AGT expression.

In one aspect, the present invention provides a method for treating a subject having an angiotensinogen-(AGT-) associated disorder, e.g., hypertension. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a nucleotide sequence comprising at least 19 contiguous nucleotides of the nucleotide sequence UGUACUCUCAUUGUGGAUGACGA (SEQ ID NO: 9) and the sense strand comprises a nucleotide sequence comprising at least 19 contiguous nucleotides of the nucleotide sequence GUCAUCCACAAUGAGAGUACA (SEQ ID NO: 10); wherein the double-stranded RNAi agent, or salt thereof, comprises at least one modified nucleotide; wherein at least one of the modifications on the nucleotides is a thermally destabilizing nucleotide modification, thereby treating the subject having the AGT-associated disorder.

In another aspect, the present invention provides a method for decreasing blood pressure level in a subject, such as a subject having an AGT-associated disorder, e.g., hypdertension. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a nucleotide sequence comprising at least 19 contiguous nucleotides of the nucleotide sequence UGUACUCUCAUUGUGGAUGACGA (SEQ ID NO: 9) and the sense strand comprises a nucleotide sequence comprising at least 19 contiguous nucleotides of the nucleotide sequence GUCAUCCACAAUGAGAGUACA (SEQ ID NO: 10); wherein the double-stranded RNAi agent, or salt thereof, comprises at least one modified nucleotide; wherein at least one of the modifications on the nucleotides is a thermally destabilizing nucleotide modification, thereby decreasing the blood pressure level in the subject.

In some embodiments, the fixed dose is administered to the subject at an interval of once a month. In other embodiments, the fixed dose is administered to the subject at an interval of once a quarter. In some embodiments, the fixed dose is administered to the subject at an interval of biannually.

In some embodiments, the subject is administered a fixed dose of about 50 mg to about 200 mg. In other embodiments, the subject is administered a fixed dose of about 200 mg to about 400 mg. In some embodiments, the subject is administered a fixed dose of about 400 mg to about 800 mg.

In some embodiments, the subject is administered a fixed dose of about 100 mg. In some embodiments, the subject is administered a fixed dose of about 200 mg. In some embodiments, the subject is administered a fixed dose of about 300 mg. In some embodiments, the subject is administered a fixed dose of about 400 mg. In some embodiments, the subject is administered a fixed dose of about 500 mg. In other embodiments, the subject is administered a fixed dose of about 600 mg. In some embodiments, the subject is administered a fixed dose of about 800 mg.

In some embodiments, the subject is administered a fixed dose of about 150 mg about once every six months.

In some embodiment, the subject is administered a fixed dose of about 300 mg about once every six months.

In some embodiment, the subject is administered a fixed dose of about 300 mg about once every three months.

In some embodiment, the subject is administered a fixed dose of about 600 mg about once every six months.

In some embodiments, the double stranded RNAi agent, or salt thereof, is administered to the subject subcutaneously or intravenously. In some embodiments, the subcutaneous administration is subcutaneous injection, e.g., subcutaneous self-administration. In other embodiments, the intravenous administration is intravenous injection.

In some embodiments, the antisense strand comprises a nucleotide sequence comprising at least 20 contiguous nucleotides of the nucleotide sequence UGUACUCUCAUUGUGGAUGACGA (SEQ ID NO: 9) and the sense strand comprises a nucleotide sequence comprising at least 20 contiguous nucleotides of the nucleotide sequence GUCAUCCACAAUGAGAGUACA (SEQ ID NO: 10).

In other embodiments, the antisense strand comprises a nucleotide sequence comprising at least 21 contiguous nucleotides of the nucleotide sequence UGUACUCUCAUUGUGGAUGACGA (SEQ ID NO: 9) and the sense strand comprises a nucleotide sequence comprising at least 20 contiguous nucleotides of the nucleotide sequence GUCAUCCACAAUGAGAGUACA (SEQ ID NO: 10).

In some embodiments, the antisense strand comprises a nucleotide sequence comprising at least 22 contiguous nucleotides of the nucleotide sequence UGUACUCUCAUUGUGGAUGACGA (SEQ ID NO: 9) and the sense strand comprises a nucleotide sequence comprising at least 20 contiguous nucleotides of the nucleotide sequence GUCAUCCACAAUGAGAGUACA (SEQ ID NO: 10).

In some embodiments, the antisense strand comprises the nucleotide sequence UGUACUCUCAUUGUGGAUGACGA (SEQ ID NO: 9) and the sense strand comprises the nucleotide sequence GUCAUCCACAAUGAGAGUACA (SEQ ID NO: 10).

In some embodiments, the antisense strand consists of the nucleotide sequence UGUACUCUCAUUGUGGAUGACGA (SEQ ID NO: 9) and the sense strand consists of the nucleotide sequence GUCAUCCACAAUGAGAGUACA (SEQ ID NO: 10).

In some embodiments, substantially all of the nucleotides of the sense strand are modified nucleotides. In other embodiments, substantially all of the nucleotides of the antisense strand are modified nucleotides.

In some embodiments, all of the nucleotides of the sense strand are modified nucleotides. In some embodiments, all of the nucleotides of the antisense strand are modified nucleotides.

In some embodiments, at least one of the nucleotide modifications is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), and a 2-0-(N-methylacetamide) modified nucleotide; and combinations thereof.

In some embodiments, at least one of the nucleotide modifications is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), and a 2-O—(N-methylacetamide) modified nucleotide; and combinations thereof.

In some embodiments, the double stranded region is 19-23 nucleotide pairs in length, 19-21 nucleotide pairs in length, 21-23 nucleotide pairs in length, or 21 nucleotide pairs in length.

In some embodiments, each strand is independently 19-23 nucleotides in length, 19-25 nucleotides in length, or 21-23 nucleotides in length. In some embodiments, the sense strand is 21 nucleotides in length, and the antisense strand is 23 nucleotides in length.

In some embodiments, at least one strand comprises a 3′ overhang of at least 1 nucleotide or a 3′ overhang of at least 2 nucleotides.

In some embodiments, the double-stranded RNAi agent, or salt thereof, further comprises at least one phosphorothioate or methylphosphonate internucleotide linkage. In some embodiments, the phosphorothioate or methylphosphonate internucleotide linkage is at the 3′-terminus of one strand. In some embodiments, the strand is the antisense strand. In other embodiments, the strand is the sense strand.

In some embodiments, the phosphorothioate or methylphosphonate internucleotide linkage is at the 5′-terminus of one strand. In some embodiments, the strand is the antisense strand. In other embodiments, the strand is the sense strand.

In some embodiments, the phosphorothioate or methylphosphonate internucleotide linkage is at the both the 5′- and 3′-terminus of one strand. In some embodiments, the strand is the antisense strand.

In one aspect, the present invention provides a method for inhibiting the expression of an angiotensinogen (AGT) gene in a subject. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a modified nucleotide sequence comprising at least 19 contiguous nucleotides of the modified nucleotide sequence usGfsuac(Tgn)cucauugUfgGfaugacsgsa (SEQ ID NO: 11) and the sense strand comprises a modified nucleotide sequence comprising at least 19 contiguous nucleotides of the modified nucleotide sequence gsuscaucCfaCfAfAfugagaguaca (SEQ ID NO: 12); wherein the chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage, thereby inhibiting the expression of the AGT gene in the subject.

In another aspect, the present invention provides a method for treating a subject that would benefit from reduction in AGT expression, e.g., a subject at risk of developing an AGT-associated disorder, e.g., hypertension. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi, or salt thereof, agent comprises a sense strand and an antisense strand forming a double stranded region, wherein the antisense strand comprises a modified nucleotide sequence comprising at least 19 contiguous nucleotides of the modified nucleotide sequence usGfsuac(Tgn)cucauugUfgGfaugacsgsa (SEQ ID NO: 11) and the sense strand comprises a modified nucleotide sequence comprising at least 19 contiguous nucleotides of the modified nucleotide sequence gsuscaucCfaCfAfAfugagaguaca (SEQ ID NO: 12); wherein chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage, thereby treating the subject that would benefit from reduction in AGT expression.

In one aspect, the present invention provides a method for treating a subject having an AGT-associated disorder, e.g., hypertension. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a modified nucleotide sequence comprising at least 19 contiguous nucleotides of the modified nucleotide sequence usGfsuac(Tgn)cucauugUfgGfaugacsgsa (SEQ ID NO: 11) and the sense strand comprises a modified nucleotide sequence comprising at least 19 contiguous nucleotides of the modified nucleotide sequence gsuscaucCfaCfAfAfugagaguaca (SEQ ID NO: 12); wherein chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage, thereby treating the subject having the AGT-associated disorder.

In another aspect, the present invention provides a method for decreasing blood pressure level in a subject. The method includes administering to the subject a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of a double-stranded ribonucleic acid (RNAi) agent, or salt thereof, wherein the double-stranded RNAi agent, or salt thereof, comprises a sense strand and an antisense strand forming a double stranded region, wherein the antisense strand comprises a modified nucleotide sequence comprising at least 19 contiguous nucleotides of the modified nucleotide sequence usGfsuac(Tgn)cucauugUfgGfaugacsgsa (SEQ ID NO: 11) and the sense strand comprises a modified nucleotide sequence comprising at least 19 contiguous nucleotides of the modified nucleotide sequence gsuscaucCfaCfAfAfugagaguaca (SEQ ID NO: 12); wherein chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage, thereby decreasing the blood pressure level in the subject.

In some embodiments, the fixed dose is administered to the subject at an interval of once a month. In other embodiments, the fixed dose is administered to the subject at an interval of once a quarter. In some embodiments, the fixed dose is administered to the subject at an interval of biannually.

In some embodiments, the subject is administered a fixed dose of about 50 mg to about 200 mg. In other embodiments, the subject is administered a fixed dose of about 200 mg to about 400 mg. In some embodiments, the subject is administered a fixed dose of about 400 mg to about 800 mg.

In some embodiments, the subject is administered a fixed dose of about 100 mg. In some embodiments, the subject is administered a fixed dose of about 200 mg. In some embodiments, the subject is administered a fixed dose of about 300 mg. In some embodiments, the subject is administered a fixed dose of about 400 mg. In some embodiments, the subject is administered a fixed dose of about 500 mg. In other embodiments, the subject is administered a fixed dose of about 600 mg. In some embodiments, the subject is administered a fixed dose of about 800 mg.

In some embodiments, the subject is administered a fixed dose of about 150 mg about once every six months.

In some embodiment, the subject is administered a fixed dose of about 300 mg about once every six months.

In some embodiment, the subject is administered a fixed dose of about 300 mg about once every three months.

In some embodiment, the subject is administered a fixed dose of about 600 mg about once every six months.

In some embodiments, the subject is administered a fixed dose of about 800 mg about once every three months.

In some embodiments, the subject is administered a fixed dose of about 800 mg about once every six months.

In some embodiments, the double stranded RNAi agent, or salt thereof, is administered to the subject subcutaneously or intravenously. In some embodiments, the subcutaneous administration is subcutaneous injection, e.g., subcutaneous self-administration. In other embodiments, the intravenous administration is intravenous injection.

In some embodiments, the antisense strand comprises a modified nucleotide sequence comprising at least 20 contiguous nucleotides of the modified nucleotide sequence usGfsuac(Tgn)cucauugUfgGfaugacsgsa (SEQ ID NO: 11) and the sense strand comprises a modified nucleotide sequence comprising at least 20 contiguous nucleotides of the modified nucleotide sequence gsuscaucCfaCfAfAfugagaguaca (SEQ ID NO: 12).

In some embodiments, the antisense strand comprises a modified nucleotide sequence comprising at least 21 contiguous nucleotides of the modified nucleotide sequence usGfsuac(Tgn)cucauugUfgGfaugacsgsa (SEQ ID NO: 11) and the sense strand comprises a modified nucleotide sequence comprising at least 20 contiguous nucleotides of the modified nucleotide sequence gsuscaucCfaCfAfAfugagaguaca (SEQ ID NO: 12).

In some embodiments, the antisense strand comprises a modified nucleotide sequence comprising at least 22 contiguous nucleotides of the modified nucleotide sequence usGfsuac(Tgn)cucauugUfgGfaugacsgsa (SEQ ID NO: 11) and the sense strand comprises a modified nucleotide sequence comprising at least 20 contiguous nucleotides of the modified nucleotide sequence gsuscaucCfaCfAfAfugagaguaca (SEQ ID NO: 12).

In some embodiments, the antisense strand comprises a modified nucleotide sequence usGfsuac(Tgn)cucauugUfgGfaugacsgsa (SEQ ID NO: 11) and the sense strand comprises a modified nucleotide sequence gsuscaucCfaCfAfAfugagaguaca (SEQ ID NO: 12).

In other embodiments, the antisense strand consists of a modified nucleotide sequence usGfsuac(Tgn)cucauugUfgGfaugacsgsa (SEQ ID NO: 11) and the sense strand consists of a modified nucleotide sequence gsuscaucCfaCfAfAfugagaguaca (SEQ ID NO: 12).

In some embodiments, the double stranded RNAi agent, or salt thereof, further comprises a ligand. In other embodiments, the ligand is conjugated to the 3′ end of the sense strand.

In some embodiments, the ligand is an N-acetylgalactosamine (GalNAc) derivative. In other embodiments, the GalNAc derivative comprises one or more GalNAc derivatives attached through a monovalent, bivalent, or trivalent branched linker.

In some embodiments, the ligand is

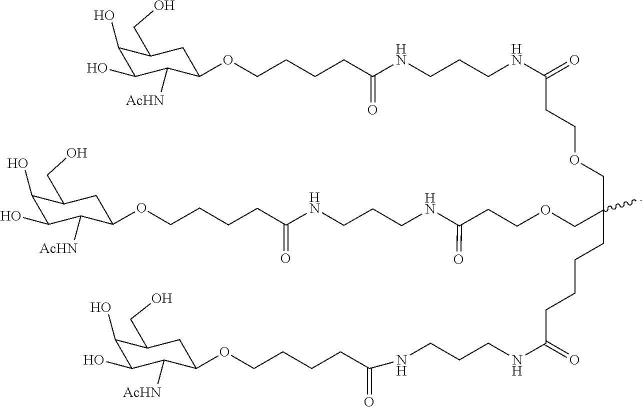 [SMILES: CC(=O)NC1C(OCCCCC(=O)NCCCNC(=O)CCCCC(C)(COCCC(=O)NCCCNC(=O)CCCCOC2CC(CO)C(O)C(O)C2NC(C)=O)COCCC(=O)NCCCNC(=O)CCCCOC2CC(CO)C(O)C(O)C2NC(C)=O)CC(CO)C(O)C1O]

In other embodiments, the 3′ end of the sense strand is conjugated to the ligand as shown in the following schematic

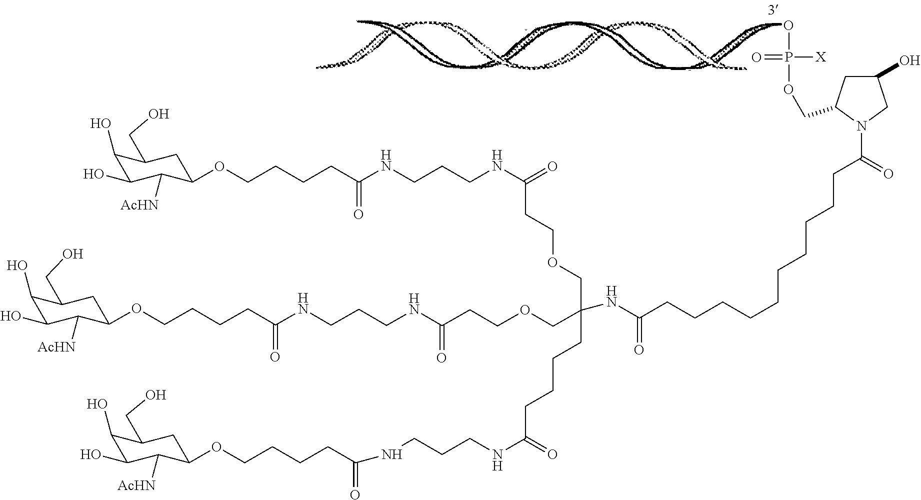

and, wherein X is O or S.

In some embodiments, the subject is a human. In some embodiments, the subject has a systolic blood pressure of at least 130 mm Hg or a diastolic blood pressure of at least 80 mm Hg. In other embodiments, the subject has a systolic blood pressure of at least 140 mm Hg or a diastolic blood pressure of at least 80 mm Hg.

In some embodiments, the subject is part of a group susceptible to salt sensitivity, is overweight, is obese, is pregnant, is planning to become pregnant, has type 2 diabetes, has type 1 diabetes, or has reduced kidney function.

In some embodiments, the disorder that would benefit from reduction in AGT expression is an AGT-associated disorder. In one embodiment, the AGT-associated disorder is hypertension. In other embodiments, the AGT-associated disorder is selected from the group consisting of high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, non-alcoholic Steatohepatitis (NASH), non-alcoholic fatty liver disease (NAFLD); glucose intolerance, type 2 diabetes, and metabolic syndrome. In one embodiment, the AGT-associated disorder is hypertension. In one embodiment, the hypertension is selected from the group consisting of high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, and hypertensive nephropathy.

In some embodiments, the blood pressure comprises systolic blood pressure and/or diastolic blood pressure.

In some embodiments, administering results in a decrease in AGT expression by at least 30%, 40% 50%, 60%, 70%, 80%, 90%, or 95%. In some embodiments, the AGT protein level in blood or serum sample of the subject is decreased by at least 30%, 40%, 50%, 60%, 70%, 80%, 90%, or 95%.

In some embodiments, administering results in a decrease in systolic blood pressure and/or diastolic blood pressure. In some embodiments, the systolic blood pressure and/or diastolic blood pressure is decreased by at least 4 mmHg, 5 mmHg, 6 mmHg, 7 mmHg, 8 mmHg, 9 mmHg, 10 mmHg or 20 mmHg.

In some embodiments, the methods further comprise administering to the subject an additional therapeutic agent for treatment of hypertension. In some embodiments, the additional therapeutic agent is selected from the group consisting of a diuretic, an angiotensin converting enzyme (ACE) inhibitor, an angiotensin II receptor antagonist, a beta-blocker, a vasodialator, a calcium channel blocker, an aldosterone antagonist, an alpha2-agonist, a renin inhibitor, an alpha-blocker, a peripheral acting adrenergic agent, a selective D1 receptor partial agonist, a nonselective alpha-adrenergic antagonist, a synthetic, a steroidal antimineralocorticoid agent; a combination of any of the foregoing; and a hypertension therapeutic agent formulated as a combination of agents. In some embodiments, the additional therapeutic agent comprises an angiotensin II receptor antagonist. In other embodiments, the angiotensin II receptor antagonist is selected from the group consisting of losartan, valsartan, olmesartan, eprosartan, irbesartan, and azilsartan.

In some embodiments, the additional therapeutic agent comprises a hypertension therapeutic agent. In some embodiments, the hypertension therapeutic agent is selected from the group consisting of olmesartan, amlodipine, and indapamide. In some embodiments, the method comprises administering to the subject a fixed dose of about 600 mg of the double stranded RNAi agent of the present invention, e.g., AD-85481, and a hypertension therapeutic agent selected from the group consisting of olmesartan, amlodipine, and indapamide.

In some embodiments, the method comprises administering to the subject a fixed dose of about 600 mg of AD-85481, and olmesartan. In some embodiments, the method comprises administering to the subject a fixed dose of about 600 mg of AD-85481, and amlodipine. In some embodiments, the method comprises administering to the subject a fixed dose of about 600 mg of AD-85481, and indapamide.

In some embodiments, the method further comprises selecting a subject whose blood pressure is not adequately controlled by standard of care antihypertensive medications.

In some embodiments, the RNAi agent is administered as a pharmaceutical composition.

In some embodiments, the RNAi agent is administered in an unbuffered solution. In some embodiments, the unbuffered solution is saline or water.

In some embodiments, the RNAi agent is administered with a buffer solution. In some embodiments, the buffer solution comprises acetate, citrate, prolamine, carbonate, or phosphate or any combination thereof. In some embodiments, the buffer solution is phosphate buffered saline (PBS).

The present invention also provides a kit for performing the methods of the invention, as described hererin. The kit comprises a) the RNAi agent, and b) instructions for use, and c) optionally, means for administering the RNAi agent to the subject.

In another aspect, the present invention also provides a pharmaceutical composition for treating an AGT-associated disorder comprising a double stranded ribonucleic acid (RNAi) agent, or salt thereof, for inhibiting expression of angiotensinogen (AGT). The pharmaceutical composition comprises a dsRNA agent comprising a sense strand and an antisense strand forming a double stranded region, wherein the antisense strand comprises a nucleotide sequence comprising at least 19 contiguous nucleotides of the nucleotide sequence of UGUACUCUCAUUGUGGAUGACGA (SEQ ID NO: 9) and the sense strand comprises a nucleotide sequence comprising at least 19 contiguous nucleotides of the nucleotide sequence of GUCAUCCACAAUGAGAGUACA (SEQ ID NO: 10); wherein no more than five of the nucleotides do not comprise a modification; wherein at least one of the nucleotide modifications is a thermally destabilizing nucleotide modification; wherein the double stranded RNAi agent is administered at a dose of at least 50 mg/dose no more than once per month, e.g. a dose of about 50 mg to about 800 mg about once per month (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg).

In certain embodiments, the antisense strand comprises a nucleotide sequence comprising at least 20 contiguous nucleotides of the nucleotide sequence UGUACUCUCAUUGUGGAUGACGA (SEQ ID NO: 9), In certain embodiments, the sense strand further comprises a nucleotide sequence comprising at least 20 contiguous nucleotides of the nucleotide sequence GUCAUCCACAAUGAGAGUACA (SEQ ID NO: 10).

In certain embodiments, the antisense strand comprises a nucleotide sequence comprising at least 21 contiguous nucleotides of the nucleotide sequence UGUACUCUCAUUGUGGAUGACGA (SEQ ID NO: 9). In certain embodiments, the sense strand further comprises the nucleotide sequence GUCAUCCACAAUGAGAGUACA (SEQ ID NO: 10).

In certain embodiments, the antisense strand a nucleotide sequence comprising comprises at least 22 contiguous nucleotides of the nucleotide sequence UGUACUCUCAUUGUGGAUGACGA (SEQ ID NO: 9), In certain embodiments, the sense strand further comprises the nucleotide sequence GUCAUCCACAAUGAGAGUACA (SEQ ID NO: 10) In certain embodiments, antisense strand comprises the nucleotide sequence UGUACUCUCAUUGUGGAUGACGA (SEQ ID NO: 9). In certain embodiments, the sense strand further comprises the nucleotide sequence GUCAUCCACAAUGAGAGUACA (SEQ ID NO:10).

In certain embodiments, the nucleotide sequence of the antisense strand consists of UGUACUCUCAUUGUGGAUGACGA (SEQ ID NO: 9). In certain embodiments, the nucleotide sequence of the sense strand further consists of GUCAUCCACAAUGAGAGUACA (SEQ ID NO: 10).

In certain embodiments, all of the nucleotides of the sense strand and all of the nucleotides of the antisense strand comprise a nucleotide modification.

In certain embodiments, at least one of the nucleotide modifications is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), and a 2-O—(N-methylacetamide) modified nucleotide; and combinations thereof. In certain embodiments, at least one of the nucleotide modifications is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), and a 2-O—(N-methylacetamide) modified nucleotide; and combinations thereof.

In certain embodiments, the nucleotide modifications are selected from the group consisting of 2′-methoxyethyl, 2′-fluoro, a 2′-deoxy-modified nucleotide, and GNA; and combinations thereof.

In certain embodiments, the double stranded region is of a length selected from: 19-23 nucleotide pairs in length, 19-21 nucleotide pairs in length, 21-23 nucleotide pairs in length, 21 nucleotide pairs in length, 19-30 nucleotide pairs in length, 19-25 nucleotide pairs in length, 23-27 nucleotide pairs in length. In certain embodiments, the double stranded region has a length of 19-21 nucleotiede pairs in length.

In certain embodiments, each strand of the double stranded RNAi or salt thereof, is independently of a length selected from 19-30 nucleotides in length 19-23 nucleotides in length, and 21-23 nucleotides in length. In certain embodiments, each strand is independently 21-23 nucleotides in length. In certain embodiments, the sense strand is 21 nucleotides in length, and the antisense strand is 23 nucleotides in length.

In certain embodiments, at least one strand comprises a 3′ overhang of at least 1 nucleotide. In certain embodiments, at least one strand comprises a 3′ overhang of at least 2 nucleotides.

In certain embodiments, the agent further comprises at least one phosphorothioate or methylphosphonate internucleotide linkage. In certain embodiments, the phosphorothioate or methylphosphonate internucleotide linkage is at the 3′-terminus of one strand. In certain embodiments, the strand is the antisense strand. In certain embodiments, the strand is the sense strand.

In certain embodiments, the phosphorothioate or methylphosphonate internucleotide linkage is at the 5′-terminus of one strand. In certain embodiments, the strand is the antisense strand. In certain embodiments, the strand is the sense strand.

In certain embodiments, the phosphorothioate or methylphosphonate internucleotide linkage is at the both the 5′- and 3′-terminus of one strand. In certain embodiments, the strand is the antisense strand.

The invention provides a pharmaceutical composition for treating an AGT-associated disorder comprising a double stranded ribonucleic acid (RNAi) agent, or salt thereof, for inhibiting expression of angiotensinogen (AGT). The pharmaceutical composition comprises a double stranded RNAi agent, or salt thereof, agent comprising a sense strand and an antisense strand forming a double stranded region, wherein the antisense strand comprises a modified nucleotide sequence comprising at least 19 contiguous nucleotides of the modified nucleotide sequence usGfsuac(Tgn)cucauugUfgGfaugacsgsa (SEQ ID NO: 11) and the sense strand comprises a modified nucleotide sequence comprising at least 19 contiguous nucleotides of the modified nucleotide sequence gsuscaucCfaCfAfAfugagaguaca (SEQ ID NO: 12); wherein chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage; and wherein the pharmaceutical composition is administered at a dose of at least 50 mg/dose no more than once per month.

In certain embodiments, the antisense strand comprises a modified nucleotide sequence comprising at least 20 contiguous nucleotides of the modified nucleotide sequence usGfsuac(Tgn)cucauugUfgGfaugacsgsa (SEQ ID NO: 11). In certain embodiments, the sense strand further comprises a modified nucleotide sequence comprising at least 20 contiguous nucleotides of the modified nucleotide sequence gsuscaucCfaCfAfAfugagaguaca (SEQ ID NO: 12).

In certain embodiments, the antisense strand comprises a modified nucleotide sequence comprising at least 21 contiguous nucleotides of the modified nucleotide sequence usGfsuac(Tgn)cucauugUfgGfaugacsgsa (SEQ ID NO: 11). In certain embodiments, the sense strand further comprises the modified nucleotide sequence gsuscaucCfaCfAfAfugagaguaca (SEQ ID NO:12).

In certain embodiments, the antisense strand comprises a modified nucleotide sequence comprising at least 22 contiguous nucleotides of the modified nucleotide sequence usGfsuac(Tgn)cucauugUfgGfaugacsgsa (SEQ ID NO: 11). In certain embodiments, the sense strand further comprises the modified nucleotide sequence gsuscaucCfaCfAfAfugagaguaca (SEQ ID NO: 12).

In certain embodiments, the antisense strand comprises the modified nucleotide sequence usGfsuac(Tgn)cucauugUfgGfaugacsgsa (SEQ ID NO: 11). In certain embodiments, the sense strand further comprises the modified nucleotide sequence gsuscaucCfaCfAfAfugagaguaca (SEQ ID NO: 12).

In certain embodiments, the modified nucleotide sequence of the antisense strand consists of usGfsuac(Tgn)cucauugUfgGfaugacsgsa (SEQ ID NO: 11). In certain embodiments, the modified nucleotide sequence of the sense strand consists of gsuscaucCfaCfAfAfugagaguaca (SEQ ID NO: 12).

In certain embodiments, the double stranded RNAi agent, or salt thereof, further comprises a ligand. In certain embodiments, the ligand is conjugated to the 3′ end of the sense strand. In certain embodiments, the ligand is an N-acetylgalactosamine (GalNAc) derivative. In certain embodiments, the GalNAc derivative comprises one or more GalNAc derivatives attached through a monovalent, bivalent, or trivalent branched linker.

In certain embodiments, ligand is

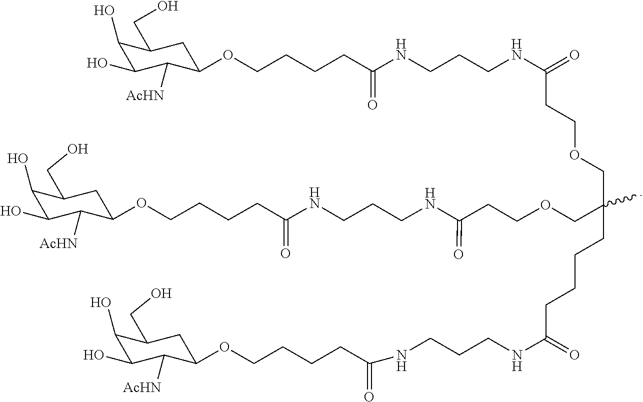 [SMILES: CC(=O)NC1C(OCCCCC(=O)NCCCNC(=O)CCCCC(C)(COCCC(=O)NCCCNC(=O)CCCCOC2CC(CO)C(O)C(O)C2NC(C)=O)COCCC(=O)NCCCNC(=O)CCCCOC2CC(CO)C(O)C(O)C2NC(C)=O)CC(CO)C(O)C1O]

In certain embodiments, the 3′ end of the sense strand is conjugated to the ligand as shown in the following schematic

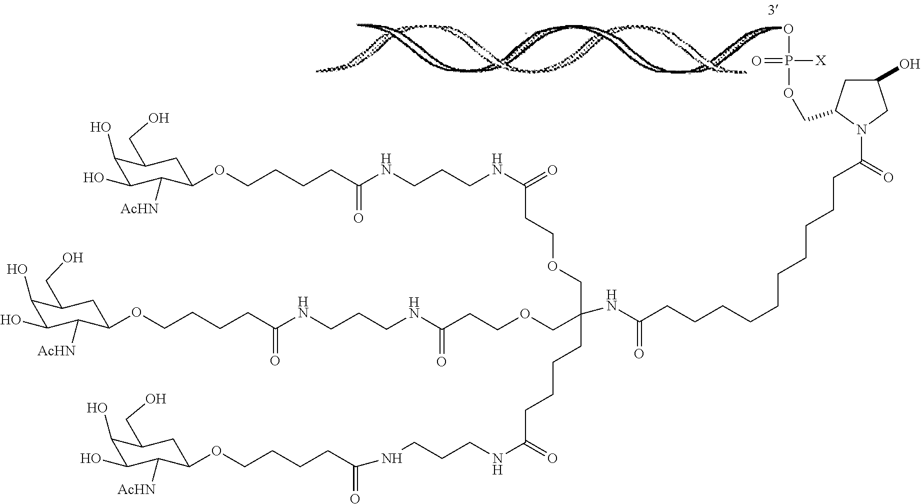

and, wherein X is O or S. In certain embodiments, the X is O.

In certain embodiments, the sense strand comprises the nucleotide sequence gsuscaucCfaCfAfAfugagaguaca wherein the 3′ end of the sense strand is conjugated to L96 (N-[tris(GalNAc-alkyl)-amidodecanoyl)]-4-hydroxyprolinol Hyp-(GalNAc-alkyl)3) and the antisense strand comprises the nucleotide sequence usGfsuac(Tgn)cucauugUfgGfaugacsgsa; wherein chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is phosphorothioate linkage.

In certain embodiments, the double stranded RNAi agent, or salt thereof, is administered at a dose of 50 mg to 500 mg per dose. In certain embodiments, the double stranded RNAi agent, or salt thereof, is administered at a dose of 50 to 400 mg per dose. In certain embodiments, the double stranded RNAi agent, or salt thereof, is administered at a dose of 50 to 300 mg per dose.

In some embodiments, the double stranded RNAi agent, or salt thereof, is administered at fixed dose of about 50 mg to about 200 mg. In other embodiments, the double stranded RNAi agent, or salt thereof, is administered at a fixed dose of about 200 mg to about 400 mg. In some embodiments, the double stranded RNAi agent, or salt thereof, is administered at a fixed dose of about 400 mg to about 800 mg.

In some embodiments, the double stranded RNAi agent, or salt thereof, is administered at a fixed dose of about 100 mg. In some embodiments, the double stranded RNAi agent, or salt thereof, is administered at a fixed dose of about 200 mg. In some embodiments, the double stranded RNAi agent, or salt thereof, is administered at a fixed dose of about 300 mg. In some embodiments, the double stranded RNAi agent, or salt thereof, is administered at a fixed dose of about 400 mg. In some embodiments, the double stranded RNAi agent, or salt thereof, is administered at a fixed dose of about 500 mg. In other embodiments, the double stranded RNAi agent, or salt thereof, is administered at fixed dose of about 600 mg. In some embodiments, the double stranded RNAi agent, or salt thereof, is administered at a fixed dose of about 800 mg.

In certain embodiments, the pharmaceutical composition is administered at a frequency of once per month to once per six months. In certain embodiments, the pharmaceutical composition is administered at a frequency of once per month to once per three months. In certain embodiments, the pharmaceutical composition is administered at a frequency of once per three months to once per six months.

In some embodiments, the pharmaceutical composition is administered to the subject at an interval of once a month. In other embodiments, the pharmaceutical composition is administered to the subject at an interval of once a quarter. In some embodiments, the pharmaceutical composition is administered to the subject at an interval of biannually.

In certain embodiments, the double stranded RNAi agent is administered at a dose of 50 to 400 mg per dose at a frequency of once per month to once per six months.

In some embodiments, the subject is administered a fixed dose of about 150 mg about once every six months.

In some embodiment, the subject is administered a fixed dose of about 300 mg about once every six months.

In some embodiment, the subject is administered a fixed dose of about 300 mg about once every three months.

In some embodiment, the subject is administered a fixed dose of about 600 mg about once every six months.

In another embodiment, the subject is administered a fixed dose of about 800 mg about once every three months.

In yet another embodiment, the subject is administered a fixed dose of about 800 mg about once every six months.

In certain embodiments, the AGT-associated disorder is selected from the group consisting of high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, non-alcoholic Steatohepatitis (NASH), non-alcoholic fatty liver disease (NAFLD); glucose intolerance, type 2 diabetes, and metabolic syndrome.

In certain embodiments, the subject has a systolic blood pressure of at least 130 mm Hg or a diastolic blood pressure of at least 80 mm Hg. In certain embodiments, the subject has a systolic blood pressure of at least 140 mm Hg and a diastolic blood pressure of at least 80 mm Hg.

In certain embodiments, the subject is part of a group susceptible to salt sensitivity, is overweight, is obese, is pregnant, is planning to become pregnant, has type 2 diabetes, or has type 1 diabetes.

In certain embodiments, the subject has an AGT-associated disorder and is further part of a group susceptible to salt sensitivity, is overweight, is obese, is pregnant, is planning to become pregnant, has type 2 diabetes, or has type 1 diabetes.

In certain embodiments, subject has reduced kidney function. In certain embodiments, the subject has an AGT-associated disorder and is further has reduced kidney function.

In certain embodiments, the pharmaceutical composition further comprises a pharmaceutically acceptable carrier.

In certain embodiments, the pharmaceutical composition is for administration by subcutaneous or intravenous injection.

The invention further provides for the use of a pharmaceutical composition of any in a method of treating an AGT-associated disorder or for use in the method of preparation of a medicament for use in a method of treating an AGT-associated disorder.

### BRIEF DESCRIPTION OF THE DRAWINGS

FIG. 1 is a graph showing percent change in serum AGT relative to AGT baseline at day 0 after a single placebo, 10 mg, 25 mg, 50 mg, 100 mg, or 200 mg subcutaneous dose of AD-85481.

FIG. 2 is a graph showing changes in systolic blood pressure (SBP) and diastolic blood pressure (DBP) at Week 8 relative to baseline after a single placebo, 10 mg, 25 mg, 50 mg, 100 mg, or 200 mg subcutaneous dose of AD-85481. The number of subjects in each group are shown along the x axis.

FIG. 3 is a schematic of the study design of the Phase II trial.

FIG. 4 is a schematic of the Phase I clinical trial study design for assessing the safety and efficacy of AD-85481 (Zilebesiran) 6 months after single-dose administration.

FIG. 5 is a graph showing percentage change in serum AGT relative to AGT baseline at Week 12 and Week 24 after a single placebo, 10 mg, 25 mg, 50 mg, 100 mg, 200 mg, 400 mg, or 800 mg subcutaneous dose of AD-85481.

FIG. 6 are graphs showing change from baseline in systolic blood pressure (SBP) and diastolic blood pressure (DBP) at week 8, week 12 and week 24 after a single placebo, 200 mg, 400 mg, or 800 mg subcutaneous dose of AD-85481. Median baseline SBP/DBP: 200 mg-139/83 mmHg; 400 mg-138/90 mmHg; 800 mg-142/88 mmHg. After Week 12, patients treated with placebo were not required to be followed. Four patients in the 200 mg dose group, 1 patient in 400 mg, and 2 patients in 800 mg received add-on antihypertensive therapy.

FIG. 7 are graphs depicting the consistent blood pressure reductions over 24 hours achieved following a single subcutaneous fixed does of AD-85481. The graph on the left depicts the change from baseline in daytime/nighttime ABPM at Week 8 following a single 200 mg, 400 mg, or 800 mg subcutaneous dose of AD-85481 or placebo. All patients at Week 8 were receiving zilebesiran only (no rescue antihypertensives). Hourly adjusted mean; daytime [9 am to 9 pm], nighttime (1 am to 6 am). Median baseline SBP/DBP: 200 mg-139/83 mmHg; 400 mg-138/90 mmHg; 800 mg-142/88 mmHg. The graph on the right depicts the 24-hour systolic blood pressure (SBP) at Week 8 after administration of a single 800 mg dose of AD-85481.

FIG. 8 is a schematic of the study design for assessing tolerability of AD-85481 (Zilebesiran) during sodium deprivation.

FIG. 9 is a graph showing changes in systolic blood pressure (SBP) and diastolic blood pressure (DBP) relative to baseline at the indicated times in patients receiving AD-85481 (Zilebesiran) or placebo following a low-salt diet or a high-salt diet.

FIG. 10 is a schematic of the study design for assessing the safety and tolerability of AD-85481 (Zilebesiran) during Irbesartan co-administration.

### DETAILED DESCRIPTION OF THE INVENTION

The present invention provides methods for inhibiting the expression of an angiotensinogen (AGT) gene. The present invention also provides methods for treating a subject having a disorder that would benefit from reduction in AGT expression, or treating an AGT-associated disorder in a subject. In addition, the present invention provides methods for decreasing blood pressure level in a subject. The methods include administering to the subject a fixed dose, e.g., about 50 mg to about 800 mg, of a double stranded RNAi agent, or salt thereof, targeting AGT, as described herein.

The following detailed description discloses methods for inhibiting the expression of an AGT gene, methods for treating subjects that would benefit from reduction of the expression of an AGT gene, e.g., subjects susceptible to or diagnosed with an AGT-associated disorder, e.g., hypertension, using an double stranded RNAi agent, or salt thereof, targeting AGT, and pharmaceutical compositions comprising fixed doses of such RNAi agents, or salt thereof, for inhibiting the expression of an AGT gene.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range. The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

In the event of a conflict between a sequence and its indicated site on a transcript or other sequence, the nucleotide sequence recited in the specification takes precedence.

In the event of a conflict between a chemical structure and a chemical name, the chemical structure takes precedence.

As used herein, “angiotensinogen,” used interchangeably with the term “AGT” refers to the well-known gene and polypeptide, also known in the art as Serpin Peptidase Inhibitor, Clade A, Member 8; Alpha-1 Antiproteinase; Antitrypsin; SERPINA8; Angiotensin I; Serpin A8; Angiotensin II; Alpha-1 Antiproteinase angiotensinogen; antitrypsin; pre-angiotensinogen2; ANHU; Serine Proteinase Inhibitor; and Cysteine Proteinase Inhibitor.

The term “AGT” includes human AGT, the amino acid and complete coding sequence of which may be found in for example, GenBank Accession No. GI:188595658 (NM_000029.3; SEQ ID NO:1); Macaca fascicularis AGT, the amino acid and complete coding sequence of which may be found in for example, GenBank Accession No. GI: 90075391 (AB170313.1: SEQ ID NO:3); mouse (Mus musculus) AGT, the amino acid and complete coding sequence of which may be found in for example, GenBank Accession No. GI: 113461997 (NM_007428.3; SEQ ID NO:5); and rat AGT (Rattus norvegicus) AGT the amino acid and complete coding sequence of which may be found in for example, for example GenBank Accession No. GI:51036672 (NM_134432; SEQ ID NO:7).

Additional examples of AGT mRNA sequences are readily available using publicly available databases, e.g., GenBank, UniProt, OMIM, and the Macaca genome project web site.

The term “AGT,” as used herein, also refers to naturally occurring DNA sequence variations of the AGT gene, such as a single nucleotide polymorphism (SNP) in the AGT gene. Exemplary SNPs may be found in the dbSNP database available at www.ncbi.nlm.nih.gov/projects/SNP/snp-_ref.cgi?geneld=183. Non-limiting examples of sequence variations within the AGT gene include, for example, those described in U.S. Pat. No. 5,589,584, the entire contents of which are incorporated herein by reference. For example, sequence variations within the AGT gene may include as a C→T at position-532 (relative to the transcription start site); a G→A at position-386; a G→A at position-218; a C→T at position-18; a G→A and a A→C at position −6 and −10; a C→T at position+10 (untanslated); a C→T at position+521 (T174M); a T→C at position+597 (P199P); a T→C at position+704 (M235T; also see, e.g., Reference SNP (refSNP) Cluster Report: rs699, available at www.ncbi.nlm.nih.gov/SNP); a A→G at position+743 (Y248C); a C→T at position+813 (N271N); a G→A at position+1017 (L339L); a C→A at position+1075 (L359M); and/or a G→A at position+1162 (V388M).

As used herein, “target sequence” refers to a contiguous portion of the nucleotide sequence of an mRNA molecule formed during the transcription of an AGT gene, including mRNA that is a product of RNA processing of a primary transcription product. The target portion of the sequence will be at least long enough to serve as a substrate for iRNA-directed cleavage at or near that portion of the nucleotide sequence of an mRNA molecule formed during the transcription of an AGT gene. In one embodiment, the target sequence is within the protein coding region of AGT.

The target sequence may be from about 19-36 nucleotides in length, e.g., preferably about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 2). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of an AGT gene in a cell, e.g., a cell within a subject, such as a mammalian subject, preferably a human subject.

In one embodiment, an RNAi agent of the invention includes a single stranded RNA that interacts with a target RNA sequence, e.g., an AGT target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., an AGT gene. Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., an AGT gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are non-ribonucleotides, e.g., a deoxyribonucleotide or a modified nucleotide. In addition, as used in this specification, an “iRNA” may include ribonucleotides with chemical modifications; an iRNA may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions, or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “iRNA” or “RNAi agent” for the purposes of this specification and claims.

The duplex region may be of any length that permits specific degradation of a desired target RNA through a RISC pathway, and may range from about 19 to 36 base pairs in length, e.g., about 19-30 base pairs in length, for example, about 19, 20, 21, 22, 23, 24, 25, 26, 27, 28, 29, 30, 31, 32, 33, 34, 35, or 36 base pairs in length, such as about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 4, 5, 6, 7, 8, 9, 10, 20, 23, or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In certain embodiments, an iRNA agent of the invention is a dsRNA, each strand of which comprises 19-23 nucleotides, that interacts with a target RNA sequence, e.g., an AGT gene, to direct cleavage of the target RNA.

In some embodiments, an iRNA of the invention is a dsRNA of 24-30 nucleotides that interacts with a target RNA sequence, e.g., an AGT target mRNA sequence, to direct the cleavage of the target RNA.

As used herein, the term “nucleotide overhang” refers to at least one unpaired nucleotide that protrudes from the duplex structure of a double stranded iRNA. For example, when a 3-end of one strand of a dsRNA extends beyond the 5-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5-end, 3-end, or both ends of either an antisense or sense strand of a dsRNA.

In certain embodiments, the antisense strand of a dsRNA has a 1-10 nucleotide, e.g., a 1, 2, 3, 4, 5, 6, 7, 8, 9, or 10 nucleotide, overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′ end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an iRNA, e.g., a dsRNA, which includes a region that is substantially complementary to a target sequence, e.g., an AGT mRNA. As used herein, the term “region of complementarity” refers to the region on the antisense strand that is substantially complementary to a sequence, for example a target sequence, e.g., an AGT nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the iRNA. In some embodiments, a double stranded RNA agent of the invention includes a nucleotide mismatch in the antisense strand. In some embodiments, a double stranded RNA agent of the invention includes a nucleotide mismatch in the sense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding an AGT gene). For example, a polynucleotide is complementary to at least a part of an AGT mRNA if the sequence is substantially complementary to a non-interrupted portion of an mRNA encoding an AGT gene.

Accordingly, in some embodiments, the sense strand polynucleotides and the antisense polynucleotides disclosed herein are fully complementary to the target AGT sequence. In other embodiments, the sense strand polynucleotides or the antisense polynucleotides disclosed herein are substantially complementary to the target AGT sequence and comprise a contiguous nucleotide sequence which is at least 80% complementary over its entire length to the equivalent region of the nucleotide sequence of any one of SEQ ID NOs:1 and 2, or a fragment of any one of SEQ ID NOs:1 and 2, such as at least 90%, or 95% complementary; or 100% complementary.

Accordingly, in some embodiments, the antisense strand polynucleotides disclosed herein are fully complementary to the target AGT sequence. In other embodiments, the antisense strand polynucleotides disclosed herein are substantially complementary to the target AGT sequence and comprise a contiguous nucleotide sequence which is at least about 90% complementary over its entire length to the equivalent region of the nucleotide sequence of SEQ ID NO:1, or a fragment of SEQ ID NO:1, such as about 90%, or about 95%, complementary. In certain embodiments, the fragment of SEQ ID NO: 1 is nucleotides 638-658 of SEQ ID NO: 1.

In certain embodiments, the nucleotide sequence of the antisense strand of an iRNA of the invention comprises at least 19 contiguous nucleotides of the nucleotide sequence UGUACUCUCAUUGUGGAUGACGA (SEQ ID NO: 9). In certain embodiments, the iRNA of the invention further comprises a sense strand comprising at least 19 contiguous nucleotides of the nucleotide sequence GUCAUCCACAAUGAGAGUACA (SEQ ID NO: 10).

In certain embodiments, the nucleotide sequence of the antisense strand of an iRNA of the invention comprises the nucletodies sequence UGUACUCUCAUUGUGGAUGACGA (SEQ ID NO: 9).

In certain embodiments, the iRNA of the invention further comprises a sense strand comprising the nucleotide sequence GUCAUCCACAAUGAGAGUACA (SEQ ID NO: 10).

In certain embodiments, the nucleotide sequence of the antisense strand of an iRNA of the invention consists of UGUACUCUCAUUGUGGAUGACGA (SEQ ID NO: 9). In certain embodiments, the iRNA of the invention further comprises a sense strand wherein the nucleotide sequence of the strand consists of the nucleotide sequence GUCAUCCACAAUGAGAGUACA (SEQ ID NO: 10).

In certain embodiments, the modified nucleotide sequence of the antisense strand of an iRNA of the invention comprises at least 19 contiguous nucleotides of the modified nucleotide sequence usGfsuac(Tgn)cucauugUfgGfaugacsgsa (SEQ ID NO: 11). In certain embodiments the iRNA of the invention further comprises a sense strand comprising a modified nucleotide sequence comprising at least 19 contiguous nucleotides of gsuscaucCfaCfAfAfugagaguaca (SEQ ID NO: 12). The chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-isomer, and s is phosphorothioate linkage; and wherein the 3′end of the sense strand is optionally covalently linked to a ligand, e.g., an N-[tris(GalNAc-alkyl)-amidodecanoyl)]-4-hydroxyprolinol (also referred to as Hyp-(GalNAc-alkyl)3 or L96).

In certain embodiments, the modified nucleotide sequence of the antisense strand of an iRNA of the invention comprises the modified nucleotide sequence usGfsuac(Tgn)cucauugUfgGfaugacsgsa (SEQ ID NO: 11). In certain embodiments the iRNA of the invention further comprises a sense strand comprising the modified nucleotide sequence gsuscaucCfaCfAfAfugagaguaca (SEQ ID NO: 12).

In certain embodiments, the modified nucleotide sequence of the antisense strand of an iRNA of the invention consists of usGfsuac(Tgn)cucauugUfgGfaugacsgsa (SEQ ID NO: 11). In certain embodiments, the iRNA of the invention further comprises a sense strand wherein the modified nucleotide sequence of the sense strand consists of the modified nucleotide sequence gsuscaucCfaCfAfAfugagaguaca (SEQ ID NO: 12).

In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In an aspect of the invention, an agent for use in the methods and compositions of the invention is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

The phrase “contacting a cell with an iRNA,” such as a dsRNA, as used herein, includes contacting a cell by any possible means. Contacting a cell with an iRNA includes contacting a cell in vitro with the iRNA or contacting a cell in vivo with the iRNA. The contacting may be done directly or indirectly. Thus, for example, the iRNA may be put into physical contact with the cell by the individual performing the method, or alternatively, the iRNA may be put into a situation that will permit or cause it to subsequently come into contact with the cell.

Contacting a cell in vitro may be done, for example, by incubating the cell with the iRNA. Contacting a cell in vivo may be done, for example, by injecting the iRNA into or near the tissue where the cell is located, or by injecting the iRNA into another area, e.g., the bloodstream or the subcutaneous space, such that the agent will subsequently reach the tissue where the cell to be contacted is located. For example, the iRNA may contain or be coupled to a ligand, e.g., GalNAc, that directs the iRNA to a site of interest, e.g., the liver. Combinations of in vitro and in vivo methods of contacting are also possible. For example, a cell may also be contacted in vitro with an iRNA and subsequently transplanted into a subject.

In certain embodiments, contacting a cell with an iRNA includes “introducing” or “delivering the iRNA into the cell” by facilitating or effecting uptake or absorption into the cell. Absorption or uptake of an iRNA can occur through unaided diffusion or active cellular processes, or by auxiliary agents or devices. Introducing an iRNA into a cell may be in vitro or in vivo. For example, for in vivo introduction, iRNA can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a cow, a pig, a horse, a goat, a rabbit, a sheep, a hamster, a guinea pig, a cat, a dog, a rat, or a mouse) that expresses the target gene, either endogenously or heterologously. In an embodiment, the subject is a human, such as a human being treated or assessed for a disease or disorder that would benefit from reduction in AGT expression; a human at risk for a disease or disorder that would benefit from reduction in AGT expression; a human having a disease or disorder that would benefit from reduction in AGT expression; or human being treated for a disease or disorder that would benefit from reduction in AGT expression as described herein. The diagnostic criteria for an AGT-associated disorder, e.g., hypertension, are provided below. In some embodiments, the subject is a female human. In other embodiments, the subject is a male human. In certain embodiments, the subject is part of a group susceptible to salt sensitivity, e.g., black or an older adult (≥65 years of age). In certain embodiments, the subject is overweight or obese, e.g., a subject that suffers from central obesity. In certain embodiments, the subject is sedentary. In certain embodiments, the subject is pregnant or planning to become pregnant. In certain embodiments, the subject has reduced kidney function. In certain embodiments the subject has type 1 diabetes. In certain embodiments, the subject has type 2 diabetes.

As used herein, the terms “treating” or “treatment” refer to a beneficial or desired result, such as reducing at least one sign or symptom of an AGT-associated disorder, e.g., hypertension in a subject. Treatment also includes a reduction of one or more sign or symptoms associated with unwanted AGT expression, e.g., angiotensin II type 1 receptor activation (AT₁R) (e.g., hypertension, chronic kidney disease, stroke, myocardial infarction, heart failure, aneurysms, peripheral artery disease, heart disease, increased oxidative stress, e.g., increased superoxide formation, inflammation, vasoconstriction, sodium and water retention, potassium and magnesium loss, renin suppression, myocyte and smooth muscle hypertrophy, increased collagen synthesis, stimulation of vascular, myocardial and renal fibrosis, increased rate and force of cardiac contractions, altered heart rate, e.g., increased arrhythmia, stimulation of plasminogen activator inhibitor 1 (PAI1), activation of the sympathetic nervous system, and increased endothelin secretion), symptoms of pregnancy-associated hypertension (e.g., preeclampsia, and eclampsia), including, but not limited to intrauterine growth restriction (IUGR) or fetal growth restriction, symptoms associated with malignant hypertension, symptoms associated with hyperaldosteronism; diminishing the extent of unwanted AT₁R activation; stabilization (i.e., not worsening) of the state of chronic AT₁R activation; amelioration or palliation of unwanted AT₁R activation (e.g., hypertension, chronic kidney disease, stroke, myocardial infarction, heart failure, aneurysms, peripheral artery disease, heart disease, increased oxidative stress, e.g., increased superoxide formation, inflammation, vasoconstriction, sodium and water retention, potassium and magnesium loss, renin suppression, myocyte and smooth muscle hypertrophy, increased collagen synthesis, stimulation of vascular, myocardial and renal fibrosis, increased rate and force of cardiac contractions, altered heart rate, e.g., increased arrhythmia, stimulation of plasminogen activator inhibitor 1 (PAI1), activation of the sympathetic nervous system, and increased endothelin secretion) whether detectable or undetectable. AGT-associated disorders can also include obesity, liver steatosis/fatty liver, e.g., non-alcoholic Steatohepatitis (NASH) and non-alcoholic fatty liver disease (NAFLD); glucose intolerance, type 2 diabetes, and metabolic syndrome. “Treatment” can also mean prolonging survival as compared to expected survival in the absence of treatment.

As used herein, “reduced kidney function” and the like can be diagnosed using any of a number of recognized criteria, e.g., glomerular filtration rate (GFR), albuminuria, creatinine, or BUN. As used herein, reduced kidney function can be transient or chronic. A GFR of at least 60 is considered to be normal. A GFR of 60 or less is indicative of reduced kidney function with a GFR of >15-60 being indicative of kidney disease, and a GFR of less than 15 is indicative of kidney failure. GFR is typically determined based on urine creatinine levels, with a higher level of creatinine indicative of lower kidney function. The presence of albumin in the urine is also indicative of decreased kidney function. The absolute level of albumin can be determined to diagnose decreased kidney function. The ratio of albumin to creatinine can also be determined to assess kidney function. A urine albumin to creatinine ratio of 30 mg/g or less is indicative of normal kidney function. A urine albumin to creatinine ratio greater than 30 mg/g is indicative of reduced kidney function.

The term “lower” in the context of the level of AGT gene expression or AGT protein production in a subject, or a disease marker or symptom refers to a statistically significant decrease in such level. The decrease can be, for example, at least 50%, 55%, 60%, 65%, 70%, 75%, 80%, 85%, 90%, or 95%, or below the level of detection for the detection method in a relevant cell or tissue, e.g., a liver cell, or other subject sample, e.g., blood or serum derived therefrom, urine. In certain embodiments, “lower” is a reduction of AGT protein in the serum after administration of one or more doses of an iRNA agent provided herein relative to AGT protein level in serum prior to administration of any doses of an iRNA agent provided herein.

As used herein, “prevention” or “preventing,” when used in reference to a disease or disorder, that would benefit from a reduction in expression of an AGT gene or production of agt protein, e.g., in a subject susceptible to an AGT-associated disorder due to, e.g., aging, genetic factors, hormone changes, diet, and a sedentary lifestyle, wherein the subject does not yet meet the diagnostic criteria for the AGT-associated disorder. As used herein, prevention can be understood as administration of an agent to a subject who does not yet meet the diagnostic criteria for the AGT-associated disorder to delay or reduce the likelihood that the subject will develop the AGT-associated disorder. As the agent is a pharmaceutical agent, it is understood that administration typically would be under the direction of a health care professional capable of identifying a subject who does not yet meet the diagnostic criteria for an AGT-associated disorder as being susceptible to developing an AGT-associated disorder. Diagnostic criteria for hypertension and risk factors for hypertension are provided below. In certain embodiments, the disease or disorder is e.g., a symptom of unwanted AT₁R activation, such as a hypertension, chronic kidney disease, stroke, myocardial infarction, heart failure, aneurysms, peripheral artery disease, heart disease, increased oxidative stress, e.g., increased superoxide formation, inflammation, vasoconstriction, sodium and water retention, potassium and magnesium loss, renin suppression, myocyte and smooth muscle hypertrophy, increased collagen synthesis, stimulation of vascular, myocardial and renal fibrosis, increased rate and force of cardiac contractions, altered heart rate, e.g., increased arrhythmia, stimulation of plasminogen activator inhibitor 1 (PAI1), activation of the sympathetic nervous system, and increased endothelin secretion. AGT-associated disorders can also include obesity, liver steatosis/fatty liver, e.g., non-alcoholic Steatohepatitis (NASH) and non-alcoholic fatty liver disease (NAFLD); glucose intolerance, type 2 diabetes, and metabolic syndrome. The likelihood of developing, e.g., hypertension, is reduced, for example, when an individual having one or more risk factors for a hypertension either fails to develop hypertension or develops hypertension with less severity relative to a population having the same risk factors and not receiving treatment as described herein. The failure to develop an AGT-associated disorder, e.g., hypertension or a delay in the time to develop hypertension by months or years is considered effective prevention. Prevention may require administration of more than one dose if the iRNA agent. Provided with appropriate methods to identify subjects at risk to develop any of the AGT-associated diseases above, the iRNA agents provided herein can be used as pharmaceutical agents for or in methods of prevention of AGT-associated diseases. Risk factors for various AGT-associated diseases are discussed below.

As used herein, the term “angiotensinogen-associated disease” or “AGT-associated disease,” is a disease or disorder that is caused by, or associated with, renin-angiotensin-aldosterone system (RAAS) activation, or a disease or disorder the symptoms of which or progression of which responds to RAAS inactivation. The term “angiotensinogen-associated disease” includes a disease, disorder, or condition that would benefit from reduction in AGT expression. Such diseases are typically associated with high blood pressure. Non-limiting examples of angiotensinogen-associated diseases include hypertension, e.g., borderline hypertension (also known as prehypertension), primary hypertension (also known as essential hypertension or idiopathic hypertension), secondary hypertension (also known as inessential hypertension), isolated systolic or diastolic hypertension, pregnancy-associated hypertension (e.g., preeclampsia, eclampsia, and post-partum preeclampsia), diabetic hypertension, resistant hypertension, refractory hypertension, paroxysmal hypertension, renovascular hypertension (also known as renal hypertension), Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy (including peripheral vascular disease), diabetic nephropathy, diabetic retinopathy, chronic heart failure, cardiomyopathy, diabetic cardiac myopathy, glomerulosclerosis, coarctation of the aorta, aortic aneurism, ventricular fibrosis, sleep apnea, heart failure (e.g., left ventricular systolic dysfunction, heart failure with decreased ejection fraction), myocardial infarction, angina, stroke, renal disease e.g., chronic kidney disease or diabetic nephropathy optionally in the context of pregnancy, renal failure, e.g., chronic renal failure, and systemic sclerosis (e.g., scleroderma renal crisis). In certain embodiments, AGT-associated disease includes intrauterine growth restriction (IUGR) or fetal growth restriction. In certain embodiments, AGT-associated disorders can also include obesity, liver steatosis/fatty liver, e.g., non-alcoholic Steatohepatitis (NASH) and non-alcoholic fatty liver disease (NAFLD); glucose intolerance, type 2 diabetes, and metabolic syndrome, and nocturnal hypotension.

Thresholds for high blood pressure and stages of hypertension are discussed in detail below.

In one embodiment, an angiotensinogen-associated disease is primary hypertension. “Primary hypertension” is a result of environmental or genetic causes (e.g., a result of no obvious underlying medical cause).

In one embodiment, an angiotensinogen-associated disease is secondary hypertension. “Secondary hypertension” has an identifiable underlying disorder which can be of multiple etiologies, including renal, vascular, and endocrine causes, e.g., renal parenchymal disease (e.g., polycystic kidneys, glomerular or interstitial disease), renal vascular disease (e.g., renal artery stenosis, fibromuscular dysplasia), endocrine disorders (e.g., adrenocorticosteroids or mineralocorticoid excess, pheochromocytoma, hyperthyroidism or hypothyroidism, growth hormone excess, hyperparathyroidism), coarctation of the aorta, or oral contraceptive use.

In one embodiment, an angiotensinogen-associated disease is pregnancy-associated hypertension, e.g., chronic hypertension of pregnancy, gestational hypertension, preeclampsia, eclampsia, preeclampsia superimposed on chronic hypertension, HELLP syndrome, and gestational hypertension (also known as transient hypertension of pregnancy, chronic hypertension identified in the latter half of pregnancy, and pregnancy-induced hypertension (PIH)). Diagnostic criteria for pregnancy-associated hypertension are provided below.

In one embodiment, an angiotensinogen-associated disease is resistant hypertension. “Resistant hypertension” is blood pressure that remains above goal (e.g., above 130 mm Hg systolic or above 90 diastolic) in spite of concurrent use of three antihypertensive agents of different classes, one of which is a thiazide diuretic. Subjects whose blood pressure is controlled with four or more medications are also considered to have resistant hypertension.

A “therapeutically-effective amount” or “prophylactically effective amount” also includes an amount of an RNAi agent that produces some desired effect at a reasonable benefit/risk ratio applicable to any treatment. The iRNA employed in the methods of the present invention may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

### II. Methods of the Invention

The present invention provides methods for inhibiting the expression of an angiotensinogen (AGT) gene. The present invention also provides methods for treating a subject that would benefit from reduction in AGT expression (such as a subject at risk of developing an AGT-associated disorder, e.g., hypertension), or treating an AGT-associated disorder, e.g., hypertension, in a subject. In addition, the present invention provides methods for decreasing blood pressure level in a subject, such as a subject having an AGT-associate disorder, e.g., hypertension. The methods include administering to the subject a fixed dose, e.g., about 50 mg to about 800 mg, of a double stranded RNAi agent targeting AGT, as described herein.

Accordingly, in one aspect, the present invention provides a methods of inhibiting the expression of an angiotensinogen (AGT) gene in a subject. The method comprises administering to the subject a fixed dose of about 50 mg to about 800 mg, e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg, of a double-stranded ribonucleic acid (RNAi) agent that inhibits expression of AGT. Values and ranges intermediate to the foregoing recited values are also intended to be part of this invention.

The term “inhibiting,” as used herein, is used interchangeably with “reducing,” “silencing,” “downregulating”, “suppressing”, and other similar terms, and includes any level of inhibition.

The phrase “inhibiting expression of an AGT” is intended to refer to inhibition of expression of any AGT gene (such as, e.g., a mouse AGT gene, a rat AGT gene, a monkey AGT gene, or a human AGT gene) as well as variants or mutants of an AGTgene. Thus, the AGT gene may be a wild-type AGT gene, a mutant AGT gene, or a transgenic AGT gene in the context of a genetically manipulated cell, group of cells, or organism.

“Inhibiting expression of an AGT gene” includes any level of inhibition of an AGT gene, e.g., at least partial suppression of the expression of an AGT gene. The expression of the AGT gene may be assessed based on the level, or the change in the level, of any variable associated with AGT gene expression, e.g., AGT mRNA level or AGT protein level. This level may be assessed in an individual cell or in a group of cells, including, for example, a sample derived from a subject. It is understood that AGT is expressed predominantly in the liver, but also in the brain, gall bladder, heart, and kidney, and is present in circulation.

Inhibition may be assessed by a decrease in an absolute or relative level of one or more variables that are associated with AGT expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the invention, expression of an AGT gene is inhibited by at least 30%, 35%, 40%, 45%, 50%, 55%, 60%, 65%, 70%, 75%, 80%, 85%, 90%, or 95%, or to below the level of detection of the assay. In preferred embodiments, expression of an AGT gene is inhibited by at least 50%. It is further understood that inhibition of AGT expression in certain tissues, e.g., in liver, without a significant inhibition of expression in other tissues, e.g., brain, may be desirable. In preferred embodiments, expression level is determined using the assay method provided in Example 2 of PCT Application No. PCT/US2019/032150 with a 10 nM siRNA concentration in the appropriate species matched cell line.

In certain embodiments, inhibition of expression in vivo is determined by knockdown of the human gene in a rodent expressing the human gene, e.g., an AAV-infected mouse expressing the human target gene (i.e., AGT), e.g., when administered a single dose at 3 mg/kg at the nadir of RNA expression.

Knockdown of expression of an endogenous gene in a model animal system can also be determined, e.g., after administration of a single dose at 3 mg/kg at the nadir of RNA expression. Such systems are useful when the nucleic acid sequence of the human gene and the model animal gene are sufficiently close such that the human iRNA provides effective knockdown of the model animal gene. RNA expression in liver is determined using the PCR methods provided in Example 2 of PCT Application No. PCT/US2019/032150.

Inhibition of the expression of an AGT gene may be manifested by a reduction of the amount of mRNA expressed by a first cell or group of cells (such cells may be present, for example, in a sample derived from a subject) in which an AGT gene is transcribed and which has or have been treated (e.g., by contacting the cell or cells with an iRNA of the invention, or by administering an iRNA of the invention to a subject in which the cells are or were present) such that the expression of an AGT gene is inhibited, as compared to a second cell or group of cells substantially identical to the first cell or group of cells but which has not or have not been so treated (control cell(s) not treated with an iRNA or not treated with an iRNA targeted to the gene of interest). In preferred embodiments, the inhibition is assessed by the method provided in Example 2 of PCT Application No. PCT/US2019/032150 using a 10 nM siRNA concentration in the species matched cell line and expressing the level of mRNA in treated cells as a percentage of the level of mRNA in control cells, using the following formula:

$$\frac{( mRNA in control cells ) - ( mRNA in treated cells )}{( mRNA in control cells )} \cdot 100 \%$$

In other embodiments, inhibition of the expression of an AGT gene may be assessed in terms of a reduction of a parameter that is functionally linked to AGT gene expression, e.g., AGT protein level in blood or serum from a subject. AGT gene silencing may be determined in any cell expressing AGT, either endogenous or heterologous from an expression construct, and by any assay known in the art.

Inhibition of the expression of an AGT protein may be manifested by a reduction in the level of the AGT protein that is expressed by a cell or group of cells or in a subject sample (e.g., the level of protein in a blood sample derived from a subject). As explained above, for the assessment of mRNA suppression, the inhibition of protein expression levels in a treated cell or group of cells may similarly be expressed as a percentage of the level of protein in a control cell or group of cells, or the change in the level of protein in a subject sample, e.g., blood or serum derived therefrom.

A control cell, a group of cells, or subject sample that may be used to assess the inhibition of the expression of an AGT gene includes a cell, group of cells, or subject sample that has not yet been contacted with an RNAi agent of the invention. For example, the control cell, group of cells, or subject sample may be derived from an individual subject (e.g., a human or animal subject) prior to treatment of the subject with an RNAi agent or an appropriately matched population control.

The level of AGT mRNA that is expressed by a cell or group of cells may be determined using any method known in the art for assessing mRNA expression. In one embodiment, the level of expression of AGT in a sample is determined by detecting a transcribed polynucleotide, or portion thereof, e.g., mRNA of the AGT gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression of AGT is determined using a nucleic acid probe. The term “probe”, as used herein, refers to any molecule that is capable of selectively binding to a specific AGT. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to AGT mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of AGT mRNA.

An alternative method for determining the level of expression of AGT in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Nat. Acad. Sci. USA 88:189-193), self sustained sequence replication (Guatelli et al. (1990) Proc. Nat. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of AGT is determined by quantitative fluorogenic RT-PCR (i.e., the TaqMan™ System). In preferred embodiments, expression level is determined by the method provided in Example 2 of PCT Application No. PCT/US2019/032150 using a 10 nM siRNA concentration in the species matched cell line.

The level of AGT protein expression may be determined using any method known in the art for the measurement of protein levels. Such methods include, for example, high performance liquid chromatography (HPLC), absorption spectroscopy, a colorimetric assays, spectrophotometric assays, flow cytometry, immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the invention are assessed by a decrease in AGT mRNA or protein level (e.g., in a liver biopsy). In certain embodiments, a puncture liver biopsy sample serves as the tissue material for monitoring the reduction in the AGT gene or protein expression.

In other embodiments, a blood sample serves as the subject sample for monitoring the reduction in the AGT protein expression.

In some embodiments of the methods of the invention, the iRNA is administered to a subject such that the iRNA is delivered to a specific site within the subject. The inhibition of expression of AGT may be assessed using measurements of the level or change in the level of AGT mRNA or agt protein in a sample derived from fluid or tissue from the specific site within the subject (e.g., liver or blood).

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

In another aspect, the present invention provides a method of treating a subject having an AGT-associated disorder, e.g., high blood pressure, e.g., hypertension. The method comprises administering to the subject a fixed dose of about 50 mg to about 800 mg, e.g., about 50-200 mg, about 200-400 mg, about 400-800 mg, e.g., about 50, 100, 150, 200, 250, 300, 350, 400, 450, 500, 550, 600, 650, 700, 750, or about 800 mg, of a double-stranded ribonucleic acid (RNAi) agent that inhibits expression of AGT. Values and ranges intermediate to the foregoing recited values are also intended to be part of this invention.

In some embodiments, the AGT-associated disorder is selected from the group consisting of high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, non-alcoholic Steatohepatitis (NASH), non-alcoholic fatty liver disease (NAFLD); glucose intolerance, type 2 diabetes, and metabolic syndrome.

In one embodiment, the AGT-associate disorder is hypertension. In one embodiment, the hypertension is borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, or hypertensive nephropathy.

In a further aspect, the present invention provides a method of treating a subject that would benefit from reduction in AGT expression. The method comprises administering to the subject a fixed dose of about 50 mg to about 800 mg, e.g., about 50-200 mg, about 200-400 mg, about 400-800 mg, e.g., about 50, 100, 150, 200, 250, 300, 350, 400, 450, 500, 550, 600, 650, 700, 750, or about 800 mg, of a double-stranded ribonucleic acid (RNAi) agent that inhibits expression of AGT. Values and ranges intermediate to the foregoing recited values are also intended to be part of this invention.

In a further aspect, the present invention provides a method of decreasing blood pressure level, e.g., systolic blood pressure and/or diastolic blood pressure, in a subject. The method comprises administering to the subject a fixed dose of about 50 mg to about 800 mg, e.g., about 50-200 mg, about 200-400 mg, about 400-800 mg, e.g., about 50, 100, 150, 200, 250, 300, 350, 400, 450, 500, 550, 600, 650, 700, 750, or about 800 mg, of a double-stranded ribonucleic acid (RNAi) agent that inhibits expression of AGT. Values and ranges intermediate to the foregoing recited values are also intended to be part of this invention.

In the methods of the invention, a cell, e.g., a cell within a subject, such as a human subject (e.g., a subject in need thereof, such as subject having an AGT-associated disorder), may be contacted with the siRNA in vitro or in vivo, i.e., the cell may be within a subject.

A cell suitable for treatment using the methods of the invention may be any cell that expresses an AGT gene, e.g., a liver cell, a brain cell, a gall bladder cell, a heart cell, or a kidney cell, but preferably a liver cell. A cell suitable for use in the methods of the invention may be a mammalian cell, e.g., a primate cell (such as a human cell, including human cell in a chimeric non-human animal, or a non-human primate cell, e.g., a monkey cell or a chimpanzee cell), or a non-primate cell. In certain embodiments, the cell is a human cell, e.g., a human liver cell. In the methods of the invention, AGT expression is inhibited in the cell by at least 30%, 35%, 40%, 45%, 50%, 55%, 60%, 65%, 70%, 75%, 80%, 85%, 90%, or 95%, or to a level below the level of detection of the assay.

In one embodiment, a dsRNA agent targeting AGT is administered to a subject such that AGT levels, e.g., in a cell, tissue, blood, urine or other tissue or fluid of the subject are reduced by at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 62%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

In another embodiment, a dsRNA agent targeting AGT is administered to a subject such that the blood pressure levels, e.g., systolic blood pressure and/or diastolic blood pressure, of the subject are reduced by at least about 1, 2, 3, 4, 5, 6, 7, 8, 9, or 10 mmHg or more.

Administration of the dsRNA agent according to the methods and uses of the invention may result in a reduction of the severity, signs, symptoms, and/or markers of such diseases or disorders in a patient with primary hyperoxaluria. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of primary hyperoxaluria may be assessed, for example, by periodic monitoring of oxalate levels in the subject being treated. Comparisons of the later measurements with the initial measurements provide a physician an indication of whether the treatment is effective. It is well within the ability of one skilled in the art to monitor efficacy of treatment or prevention by measuring such a parameter, or any combination of parameters. In connection with the administration of a dsRNA agent targeting AGT or pharmaceutical composition thereof, “effective against” primary hyperoxaluria indicates that administration in a clinically appropriate manner results in a beneficial effect for at least a statistically significant fraction of patients, such as improvement of symptoms, a cure, a reduction in disease, extension of life, improvement in quality of life, or other effect generally recognized as positive by medical doctors familiar with treating primary hyperoxaluria and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given dsRNA agent drug or formulation of that drug can also be judged using an experimental animal model for the given disease as known in the art. When using an experimental animal model, efficacy of treatment is evidenced when a statistically significant reduction in a marker or symptom is observed.

Any positive change resulting in e.g., lessening of severity of disease measured using the appropriate scale, represents adequate treatment using a dsRNA agent or dsRNA agent formulation as described herein.

The in vivo methods of the invention may include administering to a subject a composition containing an iRNA, where the iRNA includes a nucleotide sequence that is complementary to at least a part of an RNA transcript of the AGT gene of the mammal to which the RNAi agent is to be administered. The composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intramuscular injection.

In some embodiments, the administration is via a depot injection. A depot injection may release the dsRNA agent in a consistent way over a prolonged time period. Thus, a depot injection may reduce the frequency of dosing needed to obtain a desired effect, e.g., a desired inhibition of AGT, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the dsRNA agent to the liver.

Other modes of administration include epidural, intracerebral, intracerebroventricular, nasal administration, intraarterial, intracardiac, intraosseous infusion, intrathecal, and intravitreal, and pulmonary. The mode of administration may be chosen based upon whether local or systemic treatment is desired and based upon the area to be treated. The route and site of administration may be chosen to enhance targeting.

The iRNA is preferably administered subcutaneously, i.e., by subcutaneous injection. One or more injections may be used to deliver the desired dose of iRNA to a subject. The injections may be repeated over a period of time.

The administration may be repeated on a regular basis. In certain embodiments, the iRNA is administered about once per month to about once per quarter, i.e., about every three months, or about once per quarter to about twice per year, i.e., about once every six months. In certain embodiments, the iRNA is administered once per month. In other embodiments, the iRNA is administered every three months, or once per quarter. In yet another embodiment, the iRNA is administered every six months or biannually.

A dsRNA agent of the invention may be administered in “naked” form, or as a “free dsRNA agent.” A naked dsRNA agent is administered in the absence of a pharmaceutical composition. The naked dsRNA agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the dsRNA agent can be adjusted such that it is suitable for administering to a subject.

Alternatively, an iRNA of the invention may be administered as a pharmaceutical composition, such as a dsRNA liposomal formulation. The RNAi agent may be administered as a pharmaceutical composition in an unbuffered solution. The unbuffered solution may comprise saline or water. Alternatively, the RNAi agent may be administered as a pharmaceutical composition in a buffer solution. The buffer solution may comprise acetate, citrate, prolamine, carbonate, or phosphate or any combination thereof. In one embodiment, the buffer solution is phosphate buffered saline (PBS).

Subjects that would benefit from an inhibition of AGT gene expression are subjects susceptible to or diagnosed with an AGT-associated disease or disorder, e.g., high blood pressure, e.g., hypertension. The subjects may have a systolic blood pressure of at least 130, 135, 140, 145, 150, 155 or 160 mmHg or a diastolic blood pressure of at least 80, 85, 90, 95, 100, 105, 110 mmHg. The subject may be susceptible to salt sensitivity, overweight, obese, pregnant, or planning to become pregnant. The subject may have type 2 diabetes, type 1 diabetes, or have reduced kidney function.

The method further comprises administering to the subject an additional therapeutic agent for treatment of hypertension. Exemplary therapeutic agents for use as a combination therapy may include, but are not limited to, a diuretic, an angiotensin converting enzyme (ACE) inhibitor, an angiotensin II receptor antagonist, a beta-blocker, a vasodialator, a calcium channel blocker, an aldosterone antagonist, an alpha2-agonist, a renin inhibitor, an alpha-blocker, a peripheral acting adrenergic agent, a selective D1 receptor partial agonist, a nonselective alpha-adrenergic antagonist, a synthetic, a steroidal antimineralocorticoid agent; a combination of any of the foregoing; and a hypertension therapeutic agent formulated as a combination of agents. In some embodiments, the additional therapeutic agent comprises an angiotensin II receptor antagonist, e.g., losartan, valsartan, olmesartan, eprosartan, irbesartan, and azilsartan.

In some embodiments, the additional therapeutic agent comprises a hypertension therapeutic agent. In some embodiments, the hypertension therapeutic agent is selected from the group consisting of olmesartan, amlodipine, and indapamide. In some embodiments, the method comprises administering to the subject a fixed dose of about 600 mg of the double stranded RNAi agent of the present invention, e.g., AD-85481, and a hypertension therapeutic agent selected from the group consisting of olmesartan, amlodipine, and indapamide.

In some embodiments, the method comprises administering to the subject a fixed dose of about 600 mg of.AD-85481, and olmesartan. In some embodiments, the method comprises administering to the subject a fixed dose of about 600 mg of.AD-85481, and amlodipine. In some embodiments, the method comprises administering to the subject a fixed dose of about 600 mg of.AD-85481, and indapamide.

Administration of the iRNA according to the methods of the invention may result prevention or treatment of an AGT associated disorder, e.g., high blood pressure, e.g., hypertension. Diagnostic criteria for various types of high blood pressure are provided below.

### III. Diagnostic Criteria, Risk Factors, and Treatments for Hypertension

Recently practice guidelines for prevention and treatment of hypertension were revised. Extensive reports were published by Reboussin et al. (Systematic Review for the 2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. J Am Coll Cardiol. 2017 Nov. 7. pii: S0735-1097(17)41517-8. doi: 10.1016/j.jacc.2017.11.004.) and Whelton et al. (2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. J Am Coll Cardiol. 2017 Nov. 7. pii: S0735-1097(17)41519-1. doi: 10.1016/j.jacc.2017.11.006.). Some highlights of the new Guidelines are provided below. However, the Guidelines should be understood as providing the knowledge of those of skill in the art regarding diagnostic and monitoring criteria and treatment for hypertension at the time of filing of this application and are incorporated herein by reference.

### A. Diagnostic Criteria

Although a continuous association exists between higher blood pressure and increased cardiovascular disease risk, it is useful to categorize blood pressure levels for clinical and public health decision making. Blood pressure can be categorized into 4 levels on the basis of average blood pressure measured in a healthcare setting (office pressures): normal, elevated, and stage 1 or 2 hypertension as shown in the table below (from Whelton et al., 2017).

| | |
|---|---|
| Systolic Blood Pressure | Diastolic Blood Pressure |

Blood Pressure Category

| | | | | | |
|---|---|---|---|---|---|
| Normal | <120 | mm Hg | and | <80 | mm Hg |
| Elevated | 120-129 | mm Hg | and | <80 | mm Hg |

Hypertension*

| | | | | | |
|---|---|---|---|---|---|
| Stage 1 | 130-139 | mm Hg | or | 80-89 | mm Hg |
| Stage 2 | ≥140 | mm Hg | or | ≥90 | mm Hg |

*Individuals with systolic blood pressure and diastolic blood pressure in 2 categories should be designated to the higher blood pressure category.

Blood pressure indicates blood pressure based on an average of ≥2 careful readings obtained on ≥2 occasions. Best practices for obtaining careful blood pressure readings are detailed in Whelton et al., 2017 and are known in the art.

This categorization differs from that previously recommended in the JNC 7 report (Chobanian et al; the National High Blood Pressure Education Program Coordinating Committee. Seventh Report of the Joint National Committee on Prevention, Detection, Evaluation, and Treatment of High Blood Pressure. Hypertension. 2003; 42:1206-52) with stage 1 hypertension now defined as a systolic blood pressure (SBP) of 130-139 or a diastolic blood pressure (DBP) of 80-89 mm Hg, and with stage 2 hypertension in the present document corresponding to stages 1 and 2 in the JNC 7 report. The rationale for this categorization is based on observational data related to the association between SBP/DBP and cardiovascular disease risk, randomized clinical trials of lifestyle modification to lower blood pressure, and randomized clinical trials of treatment with antihypertensive medication to prevent cardiovascular disease.

The increased risk of cardiovascular disease among adults with stage 2 hypertension is well established. An increasing number of individual studies and meta-analyses of observational data have reported a gradient of progressively higher cardiovascular disease risk going from normal blood pressure to elevated blood pressure and stage 1 hypertension. In many of these meta-analyses, the hazard ratios for coronary heart disease and stroke were between 1.1 and 1.5 for the comparison of SBP/DBP of 120-129/80-84 mm Hg versus <120/80 mm Hg and between 1.5 and 2.0 for the comparison of SBP/DBP of 130-139/85-89 mm Hg versus <120/80 mm Hg. This risk gradient was consistent across subgroups defined by sex and race/ethnicity. The relative increase in cardiovascular disease risk associated with higher blood pressure was attenuated but still present among older adults. Lifestyle modification and pharmacological antihypertensive treatment are recommended for individuals with elevated blood pressure and stages 1 and 2 hypertension. Clinical benefit can be obtained by a reduction of the stage of elevated blood pressure, even if blood pressure is not normalized by a treatment.

### B. Risk Factors

Hypertension is a complex disease that results from a combination of factors including, but not limited to, genetics, lifestyle, diet, and secondary risk factors. Hypertension can also be associated with pregnancy. It is understood that due to the complex nature of hypertension, it is understood that multiple interventions may be required for treatment of hypertension. Moreover, non-pharmacological interventions, including modification of diet and lifestyle, can be useful for the prevention and treatment of hypertension. Further, an intervention may provide a clinical benefit without fully normalizing blood pressure in an individual.

1. Genetic Risk Factors Several monogenic forms of hypertension have been identified, such as glucocorticoid-remediable aldosteronism, Liddle's syndrome, Gordon's syndrome, and others in which single-gene mutations fully explain the pathophysiology of hypertension, these disorders are rare. The current tabulation of known genetic variants contributing to blood pressure and hypertension includes more than 25 rare mutations and 120 single nucleotide polymorphisms. However, although genetic factors may contribute to hypertension in some individuals, it is estimated that genetic variation accounts for only about 3.5% of blood pressure variability.
2. Diet and Alcohol Consumption Common environmental and lifestyle risk factors leading to hypertension include poor diet, insufficient physical activity, and excess alcohol consumption. These factors can lead to a person to become overweight or obese, further increasing the likelihood of developing or exacerbating hypertension. Elevated blood pressure is even more strongly correlated with increased waist-to-hip ratio or other measures of central fat distribution. Obesity at a young age and ongoing obesity is strongly correlated with hypertension later in life. Achieving a normal weight can reduce the risk of developing high blood pressure to that of a person who has never been obese.

Intake of sodium, potassium, magnesium, and calcium can also have a significant effect on blood pressure. Sodium intake is positively correlated with blood pressure and accounts for much of the age-related increase in blood pressure. Certain groups are more sensitive to increased sodium consumption than others including black and older adults (≥65 years old), and those with a higher level of blood pressure or comorbidities such as chronic kidney disease, diabetes mellitus, or metabolic syndrome. In aggregate, these groups constitute more than half of all US adults. Salt sensitivity may be a marker for increased cardiovascular disease and all-cause mortality, independent of blood pressure. Currently, techniques for recognition of salt sensitivity are impractical in a clinical setting. Therefore, salt sensitivity is best considered as a group characteristic.

Potassium intake is inversely related to blood pressure and stroke, and a higher level of potassium seems to blunt the effect of sodium on blood pressure. A lower sodium-potassium ratio is associated with a lower blood pressure than that noted for corresponding levels of sodium or potassium on their own. A similar observation has been made for risk of cardiovascular disease.

Alcohol consumption has long been associated with high blood pressure. In the US, it has been estimated that alcohol consumption accounts for about 10% of the population burden of hypertension, with the burden being greater in men than women.

It is understood that changes in diet or alcohol consumption can be an aspect of prevention or treatment of hypertension.

### 3. Physical Activity

There is a well-established inverse correlation between physical activity/physical fitness and blood pressure levels. Even modest levels of physical activity have been demonstrated to be beneficial in decreasing hypertension.

It is understood that an increase in physical activity can be an aspect of prevention or treatment of hypertension.

4. Secondary Risk Factors Secondary hypertension can underlie severe elevation of blood pressure, pharmacologically resistant hypertension, sudden onset of hypertension, increased blood pressure in patients with hypertension previously controlled on drug therapy, onset of diastolic hypertension in older adults, and target organ damage disproportionate to the duration or severity of the hypertension. Although secondary hypertension should be suspected in younger patients (<30 years of age) with elevated blood pressure, it is not uncommon for primary hypertension to manifest at a younger age, especially in blacks, and some forms of secondary hypertension, such as renovascular disease, are more common at older age (≥65 years of age). Many of the causes of secondary hypertension are strongly associated with clinical findings or groups of findings that suggest a specific disorder. In such cases, treatment of the underlying condition may resolve the findings of elevated blood pressure without administering agents typically used for the treatment of hypertension.
5. Pregnancy Pregnancy is a risk factor for high blood pressure, and high blood pressure during pregnancy is a risk factor for cardiovascular disease and hypertension later in life. A Report on pregnancy associated hypertension was published in 2013 by the American College of Obstetrics and Gynecology (ACOG) (American College of Obstetricians and Gynecologists, Task Force on Hypertension in Pregnancy. Hypertension in pregnancy. Report of the American College of Obstetricians and Gynecologists' Task Force on Hypertension in Pregnancy. Obstet Gynecol. 2013; 122:1122-31). Some highlights of the Report are provided below. However, the Report should be understood as providing the knowledge of those of skill in the art regarding diagnostic and monitoring criteria and treatment for hypertension in pregnancy at the time of filing of this application and are incorporated herein by reference.’

The diagnostic criteria for preeclampsia are provided in the table below (from Table 1 of the ACOG report, 2013).

| | |
|---|---|
| Blood Pressure | ≥140 mm Hg diastolic or ≥90 mm Hg diastolic on two occasions at least 4 |
| | hours apart after 20 weeks of gestation in a woman with a previously normal |
| | blood pressure |
| | ≥160 mm Hg systolic or ≥110 mm Hg diastolic, hypertension can be |
| | confirmed within a short interval (minutes) to facilitate timely |
| | antihypertensive therapy |

and

| | |
|---|---|
| Proteinurea | ≥300 mg per 24-hour urine collection (or this amount extrapolated for a |
| | timed collection) |
| | Or |
| | Protein/creatinine ratio ≥0.3 (each measured as mg/dL) |

Or in the absence of proteinurea, new onset of hypertension
with the new onset of an of the following:

| | |
|---|---|
| Thrombocytopenia | Platelet count ≤100,000/microliter |
| Renal insufficiency | Serum creatinine concentration ≥1.1 mg/dL or a doubling of the serum |
| | creatinine concentration in the absence of other renal disease |
| Impaired liver | Elevated blood concentrations if liver transaminases to twice normal |
| function | concentration |
| Pulmonary edema | |
| Cerebral or visual | |
| symptoms | |

Blood Pressure management during pregnancy is complicated by the fact that many commonly used antihypertensive agents, including ACE inhibitors and ARBs, are contraindicated during pregnancy because of potential harm to the fetus. The goal of antihypertensive treatment during pregnancy includes prevention of severe hypertension and the possibility of prolonging gestation to allow the fetus more time to mature before delivery. A review of treatment for pregnancy-associated severe hypertension found insufficient evidence to recommend specific agents; rather, clinician experience was recommended in this setting (Duley L, Meher S, Jones L. Drugs for treatment of very high blood pressure during pregnancy. Cochrane Database Syst Rev. 2013;7:CD001449.).

### C. Treatments

Treatment of high blood pressure is complex as it is frequently present with other comorbidities, often including reduced renal function, for which the subject may also be undergoing treatment. Clinicians managing adults with high blood pressure should focus on overall patient health, with a particular emphasis on reducing the risk of future adverse cardiovascular disease outcomes. All patient risk factors need to be managed in an integrated fashion with a comprehensive set of nonpharmacological and pharmacological strategies. As patient blood pressure and risk of future cardiovascular disease events increase, blood pressure management should be intensified.

Whereas treatment of high blood pressure with blood pressure-lowering medications on the basis of blood pressure level alone is considered cost effective, use of a combination of absolute cardiovascular disease risk and blood pressure level to guide such treatment is more efficient and cost effective at reducing risk of cardiovascular disease than is use of blood pressure level alone. Many patients started on a single agent will subsequently require ≥2 drugs from different pharmacological classes to reach their blood pressure goals. Knowledge of the pharmacological mechanisms of action of each agent is important. Drug regimens with complementary activity, where a second antihypertensive agent is used to block compensatory responses to the initial agent or affect a different pressor mechanism, can result in additive lowering of blood pressure. For example, thiazide diuretics may stimulate the renin-angiotensin-aldosterone system. By adding an ACE inhibitor or ARB to the thiazide, an additive blood pressure lowering effect may be obtained. Use of combination therapy may also improve adherence. Several 2- and 3-fixed-dose drug combinations of antihypertensive drug therapy are available, with complementary mechanisms of action among the components.

Table 18 from Whelton et al. 2017 listing oral antihypertensive drugs is provided below. Classes of therapeutic agents for the treatment of high blood pressure and drugs that fall within those classes are provided. Dose ranges, frequencies, and comments are also provided.

| | | | | |
|---|---|---|---|---|
| | | Usual Dose, | | |
| | | Range | Daily | |
| Class | Drug | (mg/d)* | Frequency | Comments |

Primary agents

| | | | | |
|---|---|---|---|---|
| Thiazide or thiazide- | Chlorthalidone | 12.5-25 | 1 | Chlorthalidone is preferred on the |
| type diuretics | Hydrochlorothiazide | 25-50 | 1 | basis of prolonged half-life and |
| | Indapamide | 1.25-2.5 | 1 | proven trial reduction of CVD. |
| | Metolazone | 2.5-10 | 1 | Monitor for hyponatremia and |
| | | | | hypokalemia, uric acid and |
| | | | | calcium levels. |
| | | | | Use with caution in patients with |
| | | | | history of acute gout unless |
| | | | | patient is on uric acid-lowering |
| | | | | therapy. |
| ACE inhibitors | Benazepril | 10-40 | 1 or 2 | Do not use in combination with |
| | Captopril | 12.5-150 | 2 or 3 | ARBs or direct renin inhibitor |
| | Enalapril | 5-40 | 1 or 2 | There is an increased risk of |
| | Fosinopril | 10-40 | 1 | hyperkalemia, especially in |
| | Lisinopril | 10-40 | 1 | patents with CKD or in those on |
| | Moexipril | 7.5-30 | 1 or 2 | K⁺ supplements or K⁺ -sparing |
| | Perindopril | 4-16 | 1 | drugs. |
| | Quinapril | 10-80 | 1 or 2 | There is a risk of acute renal |
| | Ramipril | 2.5-10 | 1 or 2 | failure in patients with severe |
| | Trandolapril | 1-4 | 1 | bilateral renal artery stenosis. |
| | | | | Do no use if patient has history of |
| | | | | angioedema with ACE inhibitors. |
| | | | | Avoid in pregnancy. |
| ARBs | Azilsartan | 40-80 | 1 | Do not use in combination with |
| | Candesartan | 8-32 | 1 | ACE inhibitors or direct renin |
| | Eprosartan | 600-800 | 1 or 2 | inhibitors. |
| | Irbesartan | 150-300 | 1 | There is an increased risk of |
| | Losartan | 50-100 | 1 or 2 | hyperkalemia in CKD or in those |
| | Olmesartan | 20-40 | 1 | on K⁺ supplements or K⁺-sparing |
| | Telmisartan | 20-80 | 1 | drugs. |
| | Valsartan | 80-320 | 1 | There is a risk of acute renal |
| | | | | failure in patients with severe |
| | | | | bilateral renal artery stenosis. |
| | | | | Do not use if patient has history of |
| | | | | angioedema with ARBs. Patients |
| | | | | with a history of angioedema with |
| | | | | an ACE inhibitor can receive an |
| | | | | ARB beginning 6 weeks after |
| | | | | ACE inhibitor is discontinued. |
| | | | | Avoid in pregnancy. |
| CCB- | Amlodipine | 2.5-10 | 1 | Avoid use in patients with |
| dihydropyridines | Felodipine | 5-10 | 1 | HFrEF; amlodipine or felodipine |
| | Isradipine | 5-10 | 2 | may be used if required |
| | Nicardipine SR | 5-20 | 1 | They are associated with dose- |
| | Nifedipine LA | 60-120 | 1 | related pedal edema, which is |
| | Nisoldipine | 30-90 | 1 | more common in women than |
| | | | | men. |
| CCB- | Diltiazem SR | 180-360 | 2 | Avoid routine use with beta |
| nondihydropyridines | Diltiazem ER | 120-480 | 1 | blockers because of increased risk |
| | Verapamil IR | 40-80 | 3 | of bradycardia and heart block. |
| | Verapamil SR | 120-480 | 1 or 2 | Do not use in patients with |
| | Verapamil-delayed | 100-480 | 1 (in the | HFrEF. |
| | onset ER (various | | evening) | There are drug interactions with |
| | forms) | | | diltiazem and verapamil (CYP3A4 |
| | | | | major substrate and moderate |
| | | | | inhibitor). |

Secondary agents

| | | | | |
|---|---|---|---|---|
| Diuretics-loop | Bumetanide | 0.5-4 | 2 | There are preferred diuretics in |
| | Furosemide | 20-80 | 2 | patients with symptomatic HF. |
| | Torsemide | 5-10 | 1 | They are preferred over thiazides |
| | | | | in patients with moderate-to- |
| | | | | severe CKD (e.g., GFR <30 |
| | | | | mL/min). |
| Diuretics- | Amiloride | 5-10 | 1 or 2 | These are monotherapy agents |
| potassium | Triamterene | 50-100 | 1 or 2 | and minimally effective |
| sparing | | | | antihypertensive agents. |
| | | | | Combination therapy of |
| | | | | potassium-sparing diuretic with a |
| | | | | thiazide can be considered in |
| | | | | patients with hypokalemia on |
| | | | | thiazide monotherapy. |
| | | | | Avoid in patients with significate |
| | | | | CKD (e.g. GFR <45 mL/min). |
| Diuretics- | Eplerenone | 50-100 | 12 | These are preferred agents in |
| aldosterone | Spironolactone | 25-100 | 1 | primary aldosteronism and |
| antagonists | | | | resistant hypertension. |
| | | | | Spironolactone is associated with |
| | | | | greater risk of gynecomastia and |
| | | | | impotence as compared with |
| | | | | eplerenone. |
| | | | | This is common add-on therapy in |
| | | | | resistant hypertension. |
| | | | | Avoid use with K⁺ supplements, |
| | | | | other K⁺-sparing diuretics, or |
| | | | | significant renal dysfunction. |
| | | | | Eplerenone often requires twice- |
| | | | | daily dosing for adequate BP |
| | | | | lowering. |
| Beta blockers- | Atenolol | 25-100 | 12 | Beta blockers are not |
| cardioselective | Betaxolol | 5-20 | 1 | recommended as first-line agents |
| | Bisoprolol | 2.5-10 | 1 | unless the patient has IHD or HF. |
| | Metoprolol | 100-400 | 2 | These are preferred in patients |
| | tartrate | | | with bronchospastic airway |
| | Metoprolol | 50-200 | 1 | disease requiring a beta blocker. |
| | succinate | | | Bisoprolol and metoprolol |
| | | | | succinate are preferred in patients |
| | | | | with HFrEF. |
| | | | | Avoid abrupt cessation. |
| Beta blockers- | Nebivolol | 5-40 | 1 | Nebivolol induces nitric oxide- |
| cardioselective and | | | | inducesd vasodilation. |
| vasodilatory | | | | Avoid abrupt cessation. |
| Beta blockers- | Nadolol | 40-120 | 1 | Avoid in patients with reactive |
| noncardioselective | Propranolol IR | 160-480 | 2 | airways disease. |
| | Propranolol LA | 80-320 | 1 | Avoid abrupt cessation. |
| Beta blockers- | Acebutolol | 200-800 | 2 | Generally avoid, especially in |
| intrinsic | Carteolol | 2.5-10 | 1 | patients with IHD or HF. |
| sympathomimetic | Penbutolol | 10-40 | 1 | Avoid abrupt cessation. |
| activity | Pindolol | 10-60 | 2 | |
| Beta blockers- | Carvedilol | 12.5-50 | 2 | Carvedilol is preferred in patients |
| combined alpha-and | Carvedilol | 20-80 | 1 | with HFrEF. |
| beta receptor | phosphate | | | Avoid abrupt cessation. |
| | Labetalol | 200-800 | 2 | |
| Direct renin | Aliskiren | 150-300 | 1 | Do not use in combination with |
| inhibitor | | | | ACE inhibitors or ARBs. |
| | | | | Aliskiren is very long acting. |
| | | | | There is an increased risk of |
| | | | | hyperkalemina in CKD or in those |
| | | | | on K⁺ supplements or K⁺-sparing |
| | | | | drugs. |
| | | | | Aliskiren may cause acute renal |
| | | | | failure in patients with severe |
| | | | | bilateral renal artery stenosis. |
| | | | | Avoid in pregnancy. |
| Alpha-1-blockers | Doxazosin | 1-8 | 1 | These are associated with |
| | Prazosin | 2-20 | 2 or 3 | orthostatic hypotension, especially |
| | Terazosin | 1-20 | 1 or 2 | in older adults. |
| | | | | They may be considered as |
| | | | | second-line agent in patients with |
| | | | | concomitant BPH. |
| Central alpha₁- | Clonidine oral | 0.1-0.8 | 2 | These are generally reserved as |
| agonist and other | Clonidine patch | 0.1-0.3 | 1 weekly | last-line because of significant |
| centrally acting | Methyldopa | 250-1000 | 2 | CNS adverse effects, especially in |
| drugs | Guanfacine | 0.5-2 | 1 | older adults. |
| | | | | Avoid abrupt discontinuation of |
| | | | | clonidine, which may induce |
| | | | | hypertensive crisis; clonidine |
| | | | | must be tapered to avoid rebound |
| | | | | hypertension. |
| Direct vasodilators | Hydralazine | 250-200 | 2 or 3 | These are associated with sodium |
| | Minoxidil | 5-100 | 1-3 | and water retention and reflex |
| | | | | tachycardia; use with a diuretic |
| | | | | and beta blocker. |
| | | | | Hydralazine is associated with |
| | | | | drug-induced lupus-like syndrome |
| | | | | at higher doses. |
| | | | | Minoxidil is associated with |
| | | | | hirsutism and required a loop |
| | | | | diurestic. Minoxidil can induce |
| | | | | pericardial effusion. |

*Dosages may vary from those listed in the FDA approved labeling (available at https://dailymed.nlm.nih.gov/dailymed/). ACE indicates angiotensin-converting enzyme; ARB, angiotensin receptor blocker; BP, blood pressure; BPH, benign prostatic hyperplasia; CCB, calcium channel blocker; CKD, chronic kidney disease; CNS, central nervous system; CVD, cardiovascular disease; ER, extended release; GFR, glomerular filtration rate; HF, heart failure; HFrEF, heart failure with reduced ejection fraction; IHD, ischemic heart disease; IR, immediate release; LA, long-acting; and SR, sustained release.
From, Chobanian et al. (2003) The JNC 7 Report. JAMA 289(19): 2560.

iv. Delivery of an iRNA Agent for Use in the Methods of the Invention

The delivery of an iRNA agent to a cell e.g., a cell within a subject, such as a human subject (e.g., a subject in need thereof, such as a subject having an AGT-associated disorder, e.g., hypertension), for use in the methods of the invention, can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In general, any method of delivering a nucleic acid molecule (in vitro or in vivo) can be adapted for use with an iRNA of the invention (see e.g., Akhtar S. and Julian R L. (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther.11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya,Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O., et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H., et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

A. Vector Encoded iRNAs for Use in the Methods of the Invention iRNA targeting the AGT gene can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

iRNA expression plasmids can be transfected into target cells as a complex with cationic lipid carriers (e.g., Oligofectamine) or non-cationic lipid-based carriers (e.g., Transit-TKO™). Multiple lipid transfections for iRNA-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are further described below.

Vectors useful for the delivery of an iRNA will include regulatory elements (promoter, enhancer, etc.) sufficient for expression of the iRNA in the desired target cell or tissue. The regulatory elements can be chosen to provide either constitutive or regulated/inducible expression.

Expression of the iRNA can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of dsRNA expression in cells or in mammals include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the iRNA transgene.

Viral vectors that contain nucleic acid sequences encoding an iRNA can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an iRNA are cloned into one or more vectors, which facilitate delivery of the nucleic acid into a patient. More detail about retroviral vectors can be found, for example, in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Lentiviral vectors contemplated for use include, for example, the HIV based vectors described in U.S. Pat. Nos. 6,143,520; 5,665,557; and 5,981,276, which are herein incorporated by reference.

Adenoviruses are also contemplated for use in delivery of iRNAs of the invention. Adenoviruses are especially attractive vehicles, e.g., for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). A suitable AV vector for expressing an iRNA featured in the invention, a method for constructing the recombinant AV vector, and a method for delivering the vector into target cells, are described in Xia H et al. (2002), Nat. Biotech. 20: 1006-1010.

Adeno-associated virus (AAV) vectors may also be used to delivery an iRNA of the invention (Walsh et al., Proc. Soc. Exp. Biol. Med. 204:289-300 (1993); U.S. Pat. No. 5,436,146). In one embodiment, the iRNA can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. Nos. 5,252,479; 5,139,941; International Patent Application No. WO 94/13788; and International Patent Application No. WO 93/24641, the entire disclosures of which are herein incorporated by reference.

Another viral vector suitable for delivery of an iRNA of the invention is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### V. Double Stranded iRNAs Agents for Use in the Methods of the Invention

Suitable double-stranded RNAi agentes for use in the methods of the invention include an antisense strand having a region of complementarity which is complementary to at least a part of an mRNA formed in the expression of an AGT gene. The region of complementarity is about 19-30 nucleotides in length (e.g., about 30, 29, 28, 27, 26, 25, 24, 23, 22, 21, 20, or 19 nucleotides in length). Upon contact with a cell expressing the AGT gene, the iRNA inhibits the expression of the AGT gene (e.g., a human, a primate, a non-primate, or a rat AGT gene) by at least 50% as assayed by, for example, a PCR or branched DNA (bDNA)-based method, or by a protein-based method, such as by immunofluorescence analysis, using, for example, western blotting or flow cytometric techniques. In preferred embodiments, inhibition of expression is determined by the qPCR method provided in the examples, especially in Example 2 of PCT Application No. PCT/US2019/032150 with the siRNA at a 10 nM concentration in an appropriate organism cell line provided therein. In preferred embodiments, inhibition of expression in vivo is determined by knockdown of the human gene in a rodent expressing the human gene, e.g., a mouse or an AAV-infected mouse expressing the human target gene, e.g., when administered a single dose at 3 mg/kg at the nadir of RNA expression. RNA expression in liver is determined using the PCR methods provided in Example 2 of PCT Application No. PCT/US2019/032150.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an AGT gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs. In certain embodiments, the duplex region is 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target AGT gene expression is not generated in the target cell by cleavage of a larger dsRNA.

A dsRNA as described herein can further include one or more single-stranded nucleotide overhangs e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but not limited to 2′-sugar modified, such as, 2-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3-terminal end of the sense strand or, alternatively, at the 3-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In some embodiments, the double-stranded RNAi agents for use in the methods of the present invention are unmodified. In other embodiments, the double-stranded RNAi agents for use in the methods of the present invention are modified, e.g., comprise chemical modifications capable of inhibiting the expression of a target gene (i.e., an AGT gene) in vivo, or enhancing stability or other beneficial characteristic of the agents. In some embodiment, the double-stranded RNAi agents comprises a thermally destabilizing nucleotide modification.

As described in more detail below, in certain aspects of the invention, substantially all of the nucleotides of an iRNA of the invention are modified. In other embodiments of the invention, all of the nucleotides of an iRNA of the invention are modified. iRNAs of the invention in which “substantially all of the nucleotides are modified” are largely but not wholly modified and can include not more than 5, 4, 3, 2, or 1 unmodified nucleotides.

A dsNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

Vi. Modified iRNAs of the Invention

In certain embodiments, the RNA of the iRNA of the invention e.g., a dsRNA, is un-modified, and does not comprise, e.g., chemical modifications or conjugations known in the art and described herein. In other embodiments, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA or substantially all of the nucleotides of an iRNA are modified, i.e., not more than 5, 4, 3, 2, or 1 unmodified nucleotides are present in a strand of the iRNA.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of iRNA compounds useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, e.g., sodium salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

Suitable RNA mimetics are contemplated for use in iRNAs provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂— ] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O—, S—, or N-alkyl; O —, S—, or N-alkenyl; O—, S— or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO] ₘCH₃, O(CH₂)_{·n}OCH₃, O(CH₂)ₙNH₂, O(CH₂) ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxy-thymine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, the entire contents of each of which are hereby incorporated herein by reference.

In some embodiments, the RNA of an iRNA can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂-O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C1-C12 alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═H₂)-2′ (and analogs thereof; see, e.g., U.S. Pat. No. 8,278,426).

Any of the foregoing bicyclic nucleosides can be prepared having one or more stereochemical sugar configurations including for example α-L-ribofuranose and β-D-ribofuranose (see WO 99/14226).

The RNA of an iRNA can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

In some embodiments, an iRNA of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of the nucleotides of an iRNA of the invention include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

A. Modified iRNAs Comprising Motifs of the Invention

In certain aspects of the invention, the double stranded RNA agents of the invention include agents with chemical modifications as disclosed, for example, in WO2013/075035, the entire contents of each of which are incorporated herein by reference. WO2013/075035 provides motifs of three identical modifications on three consecutive nucleotides into a sense strand or antisense strand of a dsRNAi agent, particularly at or near the cleavage site. In some embodiments, the sense strand and antisense strand of the dsRNAi agent may otherwise be completely modified. The introduction of these motifs interrupts the modification pattern, if present, of the sense or antisense strand. The dsRNAi agent may be optionally conjugated with a GalNAc derivative ligand, for instance on the sense strand.

More specifically, when the sense strand and antisense strand of the double stranded RNA agent are completely modified to have one or more motifs of three identical modifications on three consecutive nucleotides at or near the cleavage site of at least one strand of a dsRNAi agent, the gene silencing activity of the dsRNAi agent was observed.

Accordingly, the invention provides double stranded RNA agents capable of inhibiting the expression of a target gene (i.e., AGT gene) in vivo. The RNAi agent comprises a sense strand and an antisense strand. Each strand of the RNAi agent may be, for example, 17-30 nucleotides in length, 25-30 nucleotides in length, 27-30 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as “dsRNAi agent.” The duplex region of a dsRNAi agent may be, for example, the duplex region can be 27-30 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 19, 20, 21, 22, 23, 24, 25, 26, and 27 nucleotides in length.

In certain embodiments, the dsRNAi agent may contain one or more overhang regions or capping groups at the 3′-end, 5′-end, or both ends of one or both strands. The overhang can be, independently, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In certain embodiments, the overhang regions can include extended overhang regions as provided above. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In certain embodiments, the nucleotides in the overhang region of the dsRNAi agent can each independently be a modified or unmodified nucleotide including, but no limited to 2′-sugar modified, such as, 2′-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand, or both strands of the dsRNAi agent may be phosphorylated. In some embodiments, the overhang region(s) contains two nucleotides having a phosphorothioate between the two nucleotides, where the two nucleotides can be the same or different. In some embodiments, the overhang is present at the 3′-end of the sense strand, antisense strand, or both strands. In some embodiments, this 3′-overhang is present in the antisense strand. In some embodiments, this 3′-overhang is present in the sense strand.

The dsRNAi agent may contain only a single overhang, which can strengthen the interference activity of the RNAi, without affecting its overall stability. For example, the single-stranded overhang may be located at the 3′-end of the sense strand or, alternatively, at the 3-end of the antisense strand. The RNAi may also have a blunt end, located at the 5′-end of the antisense strand (or the 3′-end of the sense strand) or vice versa. Generally, the antisense strand of the dsRNAi agent has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In certain embodiments, the dsRNAi agent is a double ended bluntmer of 19 nucleotides in length, wherein the sense strand contains at least one motif of three 2′-F modifications on three consecutive nucleotides at positions 7, 8, 9 from the 5′ end. The antisense strand contains at least one motif of three 2′-O-methyl modifications on three consecutive nucleotides at positions 11, 12, 13 from the 5′ end.

In other embodiments, the dsRNAi agent is a double ended bluntmer of 20 nucleotides in length, wherein the sense strand contains at least one motif of three 2′-F modifications on three consecutive nucleotides at positions 8, 9, 10 from the 5′ end. The antisense strand contains at least one motif of three 2′-O-methyl modifications on three consecutive nucleotides at positions 11, 12, 13 from the 5′ end.

In yet other embodiments, the dsRNAi agent is a double ended bluntmer of 21 nucleotides in length, wherein the sense strand contains at least one motif of three 2′-F modifications on three consecutive nucleotides at positions 9, 10, 11 from the 5′ end. The antisense strand contains at least one motif of three 2′-O-methyl modifications on three consecutive nucleotides at positions 11, 12, 13 from the 5′ end.

In certain embodiments, the dsRNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′ end; the antisense strand contains at least one motif of three 2′-O-methyl modifications on three consecutive nucleotides at positions 11, 12, 13 from the 5′ end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In certain embodiments, every nucleotide in the sense strand and the antisense strand of the dsRNAi agent, including the nucleotides that are part of the motifs are modified nucleotides. In certain embodiments each residue is independently modified with a 2′-O-methyl or 3′-fluoro, e.g., in an alternating motif. Optionally, the dsRNAi agent further comprises a ligand (preferably GalNAc₃).

In certain embodiments, the dsRNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In certain embodiments, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein Dicer cleavage of the dsRNAi agent preferentially results in an siRNA comprising the 3′-end of the second strand, thereby reducing expression of the target gene in the mammal. Optionally, the dsRNAi agent further comprises a ligand.

In certain embodiments, the sense strand of the dsRNAi agent contains at least one motif of three identical modifications on three consecutive nucleotides, where one of the motifs occurs at the cleavage site in the sense strand.

In certain embodiments, the antisense strand of the dsRNAi agent can also contain at least one motif of three identical modifications on three consecutive nucleotides, where one of the motifs occurs at or near the cleavage site in the antisense strand.

For a dsRNAi agent having a duplex region of 19-23 nucleotide in length, the cleavage site of the antisense strand is typically around the 10, 11, and 12 positions from the 5′-end. Thus the motifs of three identical modifications may occur at the 9, 10, 11 positions; the 10, 11, 12 positions; the 11, 12, 13 positions; the 12, 13, 14 positions; or the 13, 14, 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the dsRNAi agent from the 5′-end.

The sense strand of the dsRNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In some embodiments, the sense strand of the dsRNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistries of the motifs are distinct from each other, and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the dsRNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In some embodiments, the wing modification on the sense strand or antisense strand of the dsRNAi agent typically does not include the first one or two terminal nucleotides at the 3′-end, 5′-end, or both ends of the strand.

In other embodiments, the wing modification on the sense strand or antisense strand of the dsRNAi agent typically does not include the first one or two paired nucleotides within the duplex region at the 3′-end, 5′-end, or both ends of the strand.

When the sense strand and the antisense strand of the dsRNAi agent each contain at least one wing modification, the wing modifications may fall on the same end of the duplex region, and have an overlap of one, two, or three nucleotides.

When the sense strand and the antisense strand of the dsRNAi agent each contain at least two wing modifications, the sense strand and the antisense strand can be so aligned that two modifications each from one strand fall on one end of the duplex region, having an overlap of one, two, or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In some embodiments, every nucleotide in the sense strand and antisense strand of the dsRNAi agent, including the nucleotides that are part of the motifs, may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′-hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′- or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′-end or ends can be phosphorylated.

It may be possible, e.g., to enhance stability, to include particular bases in overhangs, or to include modified nucleotides or nucleotide surrogates, in single strand overhangs, e.g., in a 5′- or 3′-overhang, or in both. For example, it can be desirable to include purine nucleotides in overhangs. In some embodiments all or some of the bases in a 3′- or 5′-overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In certain embodiments, the Nₐ or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′ to 3′ of the strand and the alternating motif in the antisense strand may start with “BABABA” from 5′ to 3′ of the strand within the duplex region. As another example, the alternating motif in the sense strand may start with “AABBAABB” from 5′ to 3′ of the strand and the alternating motif in the antisense strand may start with “BBAABBAA” from 5′ to 3′ of the strand within the duplex region, so that there is a complete or partial shift of the modification patterns between the sense strand and the antisense strand.

In some embodiments, the dsRNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand or antisense strand interrupts the initial modification pattern present in the sense strand or antisense strand. This interruption of the modification pattern of the sense or antisense strand by introducing one or more motifs of three identical modifications on three consecutive nucleotides to the sense or antisense strand may enhance the gene silencing activity against the target gene.

In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotides, and “Nₐ” and “N_{b}” represent a modification to the nucleotide next to the motif “YYY” that is different than the modification of Y, and where Nₐ and N_{b} can be the same or different modifications. Alternatively, Nₐ or N_{b} may be present or absent when there is a wing modification present.

The iRNA may further comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′ end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In one embodiment, the dsRNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I═inosine). Mismatches, e.g., non-canonical or other than canonical pairings (as described elsewhere herein) are preferred over canonical (A:T, A:U, G:C) pairings; and pairings which include a universal base are preferred over canonical pairings.

In certain embodiments, the dsRNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In certain embodiments, the nucleotide at the 1 position within the duplex region from the 5′-end in the antisense strand is selected from A, dA, dU, U, and dT. Alternatively, at least one of the first 1, 2, or 3 base pair within the duplex region from the 5′-end of the antisense strand is an AU base pair. For example, the first base pair within the duplex region from the 5′-end of the antisense strand is an AU base pair.

In other embodiments, the nucleotide at the 3′-end of the sense strand is deoxy-thymine (dT) or the nucleotide at the 3′-end of the antisense strand is deoxy-thymine (dT). For example, there is a short sequence of deoxy-thymine nucleotides, for example, two dT nucleotides on the 3′-end of the sense, antisense strand, or both strands.

In certain embodiments, the sense strand sequence may be represented by formula (I):

5′ nₚ-Nₐ-(X X X )ᵢ-N_{b}-Y Y Y -N_{b}-(Z Z Z )ⱼ-Nₐ-n_{q} 3′
(I)

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY, and ZZZ each independently represent one motif of three identical modifications on three consecutive nucleotides. Preferably YYY is all 2′-F modified nucleotides.

In some embodiments, the Nₐ or N_{b} comprises modifications of alternating pattern.

In some embodiments, the YYY motif occurs at or near the cleavage site of the sense strand. For example, when the dsRNAi agent has a duplex region of 17-23 nucleotides in length, the YYY motif can occur at or the vicinity of the cleavage site (e.g.: can occur at positions 6, 7, 8; 7, 8, 9; 8, 9, 10; 9, 10, 11; 10, 11,12; or 11, 12, 13) of the sense strand, the count starting from the first nucleotide, from the 5′-end; or optionally, the count starting at the first paired nucleotide within the duplex region, from the 5′-end.

In one embodiment, i is 1 and j is 0, or i is 0 and j is 1, or both i and j are 1. The sense strand can therefore be represented by the following formulas:

5′ nₚ-Nₐ-YYY-N_{b}-ZZZ-Nₐ-n_{q} 3′ (Ib);
5′ nₚ-Nₐ-XXX-N_{b}-YYY-Nₐ-n_{q} 3′ (Ic);
or
5′ nₚ-Nₐ-XXX-N_{b}-YYY-N_{b}-ZZZ-Nₐ-n_{q} 3′ (Id).

When the sense strand is represented by formula (Ib), N_{b} represents an oligonucleotide sequence comprising 0-10, 0-7, 0-5, 0-4, 0-2, or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2, or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2, or 0 modified nucleotides. Preferably, N_{b} is 0, 1, 2, 3, 4, 5, or 6 Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

5′ nₚ-Nₐ-YYY - Nₐ-n_{q} 3′ (Ia).

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

5′ n_{q}′-Nₐ′-(Z′Z′Z′)ₖ-N_{b}′-Y′Y′Y′-N_{b}′-(X′X′X′)ₗ-N′ₐ-
nₚ′ 3′ (II)

wherein:
k and l are each independently 0 or 1;
p′ and q′ are each independently 0-6;
each Nₐ′ independently represents an oligonucleotide sequence comprising 0-25 modified nucleotides, each sequence comprising at least two differently modified nucleotides;
each N_{b}′ independently represents an oligonucleotide sequence comprising 0-10 modified nucleotides;
each nₚ′ and n_{q}′ independently represent an overhang nucleotide;
wherein N_{b}′ and Y′ do not have the same modification; and
X′X′X′, Y′Y′Y′, and Z′Z′Z′ each independently represent one motif of three identical modifications on three consecutive nucleotides.

In some embodiments, the Nₐ′ or N_{b}′ comprises modifications of alternating pattern.

The Y′Y′Y′ motif occurs at or near the cleavage site of the antisense strand. For example, when the dsRNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense strand, with the count starting from the first nucleotide, from the 5′-end; or optionally, the count starting at the first paired nucleotide within the duplex region, from the 5′-end. Preferably, the Y′Y′Y′ motif occurs at positions 11, 12, 13.

In certain embodiments, Y′Y′Y′ motif is all 2′-OMe modified nucleotides.

In certain embodiments, k is 1 and 1 is 0, or k is 0 and 1 is 1, or both k and 1 are 1.

The antisense strand can therefore be represented by the following formulas:

5′ n_{q′}-Nₐ′-Z′Z′Z′-N_{b}′-Y′Y′Y′-Nₐ′-n_{p′} 3′ (IIb);
5′ n_{q′}-Nₐ′-Y′Y′Y′-N_{b}′-X′X′X′-n_{p′} 3′ (IIc);
or
5′ n_{q′}-Nₐ′- Z′Z′Z′-N_{b}′-Y′Y′Y′-N_{b}′- X′X′X′-Nₐ′-n_{p′}
3′ (IId).

When the antisense strand is represented by formula (IIb), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5, or 6.

In other embodiments, k is 0 and 1 is 0 and the antisense strand may be represented by the formula:

5′ n_{p′}-N_{a′} - Y′Y′Y′ - N_{a′}-n_{q′} 3′ (Ia).

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other. Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′, and Z′, in particular, may represent a 2′-O-methyl modification or a 2′-fluoro modification.

In some embodiments, the sense strand of the dsRNAi agent may contain YYY motif occurring at 9, 10, and 11 positions of the strand when the duplex region is 21 nt, the count starting from the first nucleotide from the 5′-end, or optionally, the count starting at the first paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In some embodiments the antisense strand may contain Y′Y′Y′ motif occurring at positions 11, 12, 13 of the strand, the count starting from the first nucleotide from the 5′-end, or optionally, the count starting at the first paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the dsRNAi agents for use in the methods of the invention may comprise a sense strand and an antisense strand, each strand having 14 to 30 nucleotides, the iRNA duplex represented by formula (III):

sense:
5′ nₚ -Nₐ-(X X X)ᵢ -N_{b}- Y Y Y -N_{b} -(ZZZ)ⱼ-Nₐ-n_{q} 3′
antisense:
3′ nₚ′-Nₐ′ -(X′X′X′)ₖ-N_{b}′-Y′Y′Y′-N_{b}′ -(Z′Z′Z′)ₗ-
Nₐ′-n_{q}′ 5′ (III)

wherein:
i, j, k, and 1 are each independently 0 or 1;
p, p′, q, and q′ are each independently 0-6;
each Nₐ and Nₐ′ independently represents an oligonucleotide sequence comprising 0-25 modified nucleotides, each sequence comprising at least two differently modified nucleotides;
each N_{b} and N_{b}′ independently represents an oligonucleotide sequence comprising 0-10 modified nucleotides;
wherein each nₚ′, nₚ, n_{q}′, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming an iRNA duplex include the formulas below:

5′ nₚ - Nₐ -Y Y Y -Nₐ-n_{q} 3′
3′ nₚ′-Nₐ′-Y′Y′Y′ -Nₐ′n_{q}′ 5′
(IIIa)
5′ nₚ -Nₐ -Y Y Y -N_{b} -ZZZ -Nₐ-n_{q} 3′
3′ nₚ′-Nₐ′-Y′Y′Y′-N_{b}′ -Z′Z′Z′-Nₐ′n_{q}′ 5′
(IIIb)
5′ nₚ-Nₐ- X X X-N_{b}-Y Y Y-Nₐ-n_{q} 3′
3′ nₚ′-Nₐ′-X′X′X′-N_{b}′-Y′Y′Y′-Nₐ′-n_{q} 5′
(IIIc)
5′ nₚ-Nₐ-X X X -N_{b}-Y Y Y -N_{b}- ZZZ -Nₐ-n_{q} 3′
3′ nₚ′-Nₐ′-X′X′X′-N_{b}′-Y′Y′Y′-N_{b}′-Z′Z′Z′-Nₐ-n_{q}′ 5′
(IIId)

When the dsRNAi agent is represented by formula (IIIa), each Nₐ independently represents an oligonucleotide sequence comprising 2-20, 2-15, or 2-10 modified nucleotides.

When the dsRNAi agent is represented by formula (IIIb), each N_{b} independently represents an oligonucleotide sequence comprising 1-10, 1-7, 1-5, or 1-4 modified nucleotides. Each Nₐ independently represents an oligonucleotide sequence comprising 2-20, 2-15, or 2-10 modified nucleotides.

When the dsRNAi agent is represented as formula (IIIc), each N_{b}, N_{b}′ independently represents an oligonucleotide sequence comprising 0-10, 0-7, 0-10, 0-7, 0-5, 0-4, 0-2, or 0 modified nucleotides. Each Nₐ independently represents an oligonucleotide sequence comprising 2-20, 2-15, or 2-10 modified nucleotides.

When the dsRNAi agent is represented as formula (IIId), each N_{b}, N_{b}′ independently represents an oligonucleotide sequence comprising 0-10, 0-7, 0-10, 0-7, 0-5, 0-4, 0-2, or Omodified nucleotides. Each Nₐ, Nₐ′ independently represents an oligonucleotide sequence comprising 2-20, 2-15, or 2-10 modified nucleotides. Each of Nₐ, Nₐ′, N_{b}, and N_{b}′ independently comprises modifications of alternating pattern.

Each of X, Y, and Z in formulas (III), (IIIa), (IIIb), (IIIc), and (IIId) may be the same or different from each other.

When the dsRNAi agent is represented by formula (III), (IIIa), (IIIb), (IIIc), and (IIId), at least one of the Y nucleotides may form a base pair with one of the Y′nucleotides. Alternatively, at least two of the Y nucleotides form base pairs with the corresponding Y′nucleotides; or all three of the Y nucleotides all form base pairs with the corresponding Y′nucleotides.

When the dsRNAi agent is represented by formula (IIIb) or (IIId), at least one of the Z nucleotides may form a base pair with one of the Z′nucleotides. Alternatively, at least two of the Z nucleotides form base pairs with the corresponding Z′nucleotides; or all three of the Z nucleotides all form base pairs with the corresponding Z′nucleotides.

When the dsRNAi agent is represented as formula (IIIc) or (IIId), at least one of the X nucleotides may form a base pair with one of the X′nucleotides. Alternatively, at least two of the X nucleotides form base pairs with the corresponding X′nucleotides; or all three of the X nucleotides all form base pairs with the corresponding X′nucleotides.

In certain embodiments, the modification on the Y nucleotide is different than the modification on the Y′nucleotide, the modification on the Z nucleotide is different than the modification on the Z′nucleotide, or the modification on the X nucleotide is different than the modification on the X′nucleotide.

In certain embodiments, when the dsRNAi agent is represented by formula (IIId), the Nₐ modifications are 2′-O-methyl or 2′-fluoro modifications. In other embodiments, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more GalNAc derivatives attached through a bivalent or trivalent branched linker (described below). In other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, when the dsRNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, the dsRNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In some embodiments, the dsRNAi agent is a multimer containing three, four, five, six, or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two dsRNAi agents represented by at least one of formulas (III), (IIIa), (IIIb), (IIIc), and (IIId) are linked to each other at the 5′ end, and one or both of the 3′ ends, and are optionally conjugated to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric iRNAs that can be used in the methods of the invention. Such publications include WO2007/091269, U.S. Pat. No. 7,858,769, WO2010/141511, WO2007/117686, WO2009/014887, and WO2011/031520 the entire contents of each of which are hereby incorporated herein by reference.

As described in more detail below, the iRNA that contains conjugations of one or more carbohydrate moieties to an iRNA can optimize one or more properties of the iRNA. In many cases, the carbohydrate moiety will be attached to a modified subunit of the iRNA. For example, the ribose sugar of one or more ribonucleotide subunits of a iRNA can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide, or polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The iRNA may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin; preferably, the acyclic group is a serinol backbone or diethanolamine backbone.

In another embodiment of the invention, an iRNA agent comprises a sense strand and an antisense strand, each strand having 14 to 40 nucleotides. The RNAi agent may be represented by formula (L):

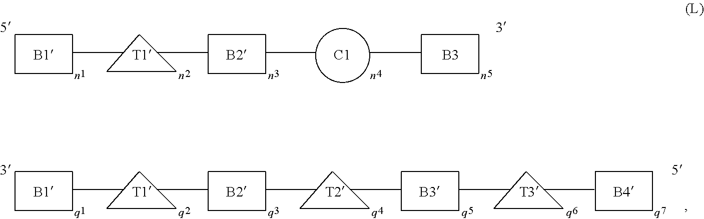

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

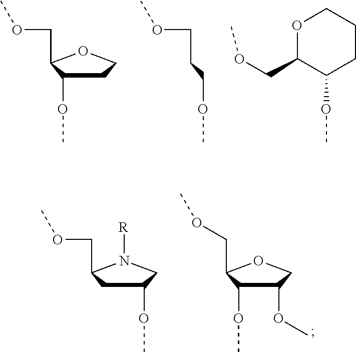

and iii) sugar modification selected from the group consisting of:

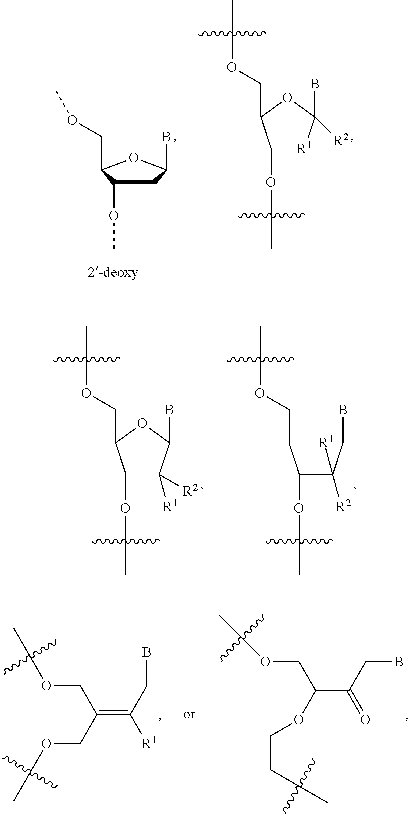

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch air is a 2′-deoxy nucleobase. In one example, the thermally destabilizing modification in C1 is GNA or

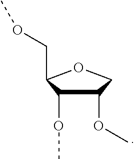

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.
n¹, n³, and q¹ are independently 4 to 15 nucleotides in length.
n⁵, q³, and q⁷ are independently 1-6 nucleotide(s) in length.
n⁴, q², and q⁶ are independently 1-3 nucleotide(s) in length; alternatively, n⁴ is 0.
q⁵ is independently 0-10 nucleotide(s) in length.
n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′- OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The RNAi agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

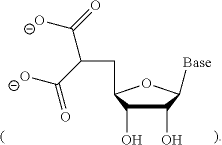

When the 5′-end phosphorus-containing group is 5′-end vinylphosphonate (5′-VP), the 5′-VP can be either 5′-E-VP isomer (i.e., trans-vinylphosphate,

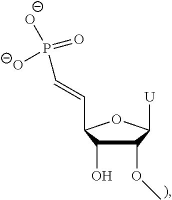

5′-Z-VP isomer (i.e., cis-vinylphosphate,

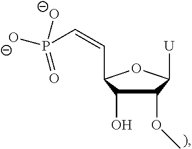

or mixtures thereof.
In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z-VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q₄ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof), and a targeting ligand.

In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
      and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
      wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a deoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
      and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
      and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
      and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
      wherein the RNAi agents have a two nucleotide overhang at the 3′-end of the antisense strand, and a blunt end at the 5′-end of the antisense strand.

In certain embodiments, the iRNA for use in the methods of the invention is an agent selected from agents listed in Table 3, Table 5, or Table 6. These agents may further comprise a ligand.

### VII. Ligands

The double-stranded RNAi gents for use in the methods of the invention may optionally be conjugated to one or more ligands, moieties or conjugates that enhance the activity, cellular distribution, or cellular uptake of the iRNA e.g., into a cell. The ligand can be attached to the sense strand, antisense strand or both strands, at the 3′-end, 5′-end or both ends. For instance, the ligand may be conjugated to the sense strand. In some embodiments, the ligand is conjugated to the 3′-end of the sense strand.

Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937). In one embodiment, the ligand is a GalNAc ligand. In particularly some embodiments, the ligand is GalNAc3. The ligands are coupled, preferably covalently, either directly or indirectly via an intervening tether.

In certain embodiments, a ligand alters the distribution, targeting, or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxol, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis.

In the ligand-conjugated iRNAs and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In other embodiments, the lipid based ligand binds HSA weakly or not at all, such that the conjugate will be preferably distributed to the kidney. Other moieties that target to kidney cells can also be used in place of, or in addition to, the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

### B. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 13). An RFGF analogue (e.g., amino acid sequence AALLPVLLAAP (SEQ ID NO: 14) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 15) and the Drosophila Antennapedia protein (RQIKIWFQNRRMKWKK (SEQ ID NO: 16) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an iRNA further comprises a carbohydrate. The carbohydrate conjugated iRNA is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri-, and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In certain embodiments, a carbohydrate conjugate for use in the compositions and methods of the invention is a monosaccharide.

In one embodiment, a carbohydrate conjugate for use in the compositions and methods of the invention is selected from the group consisting of:

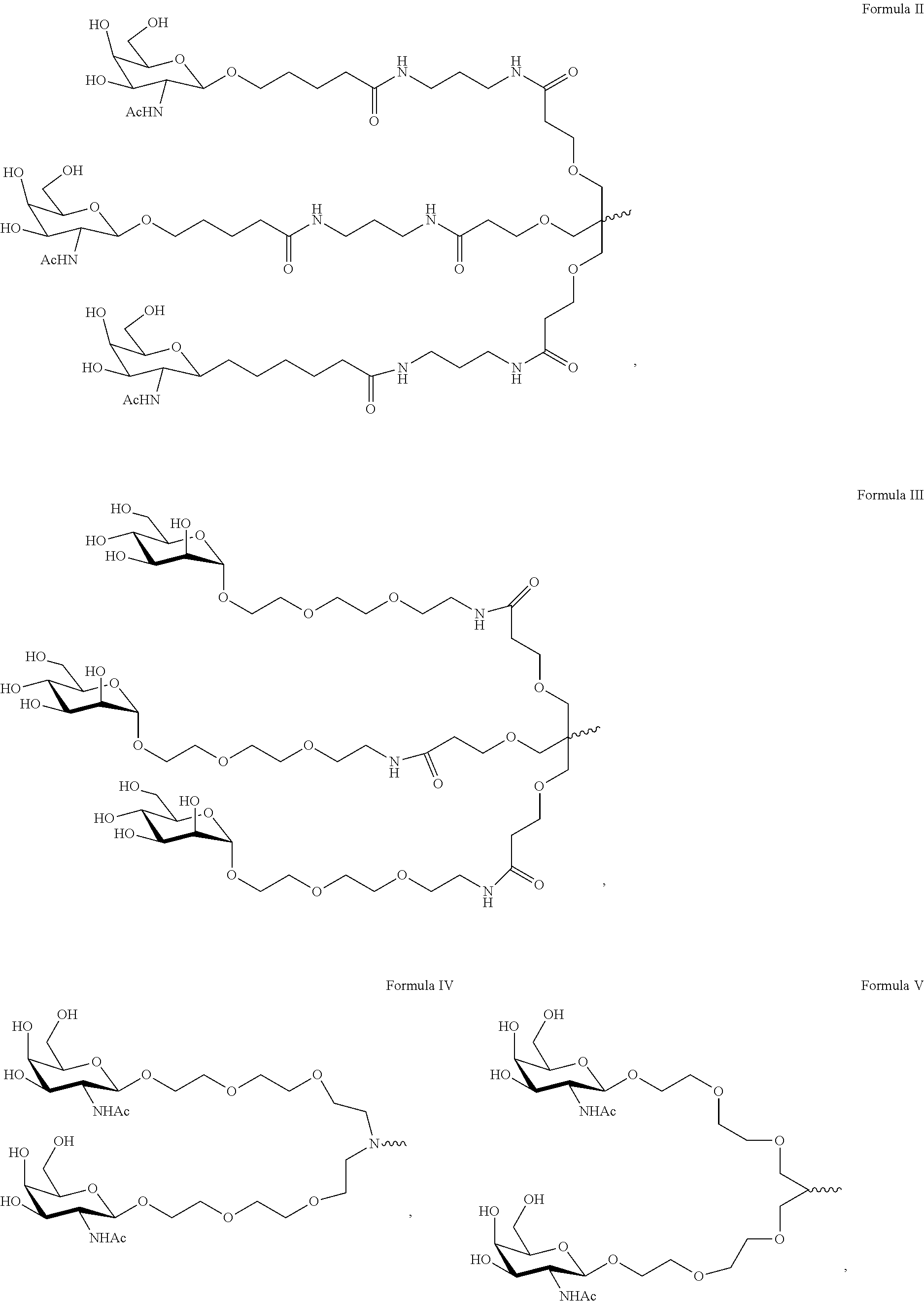

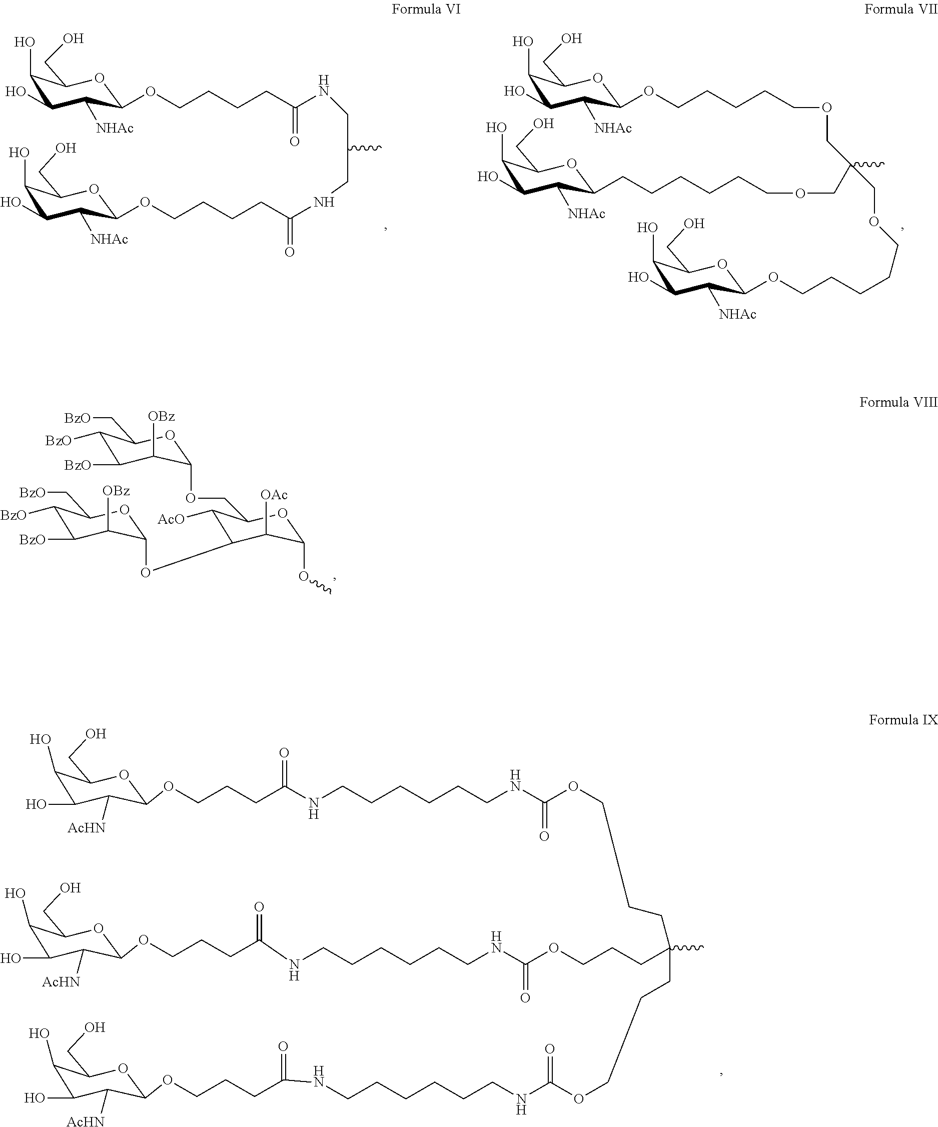

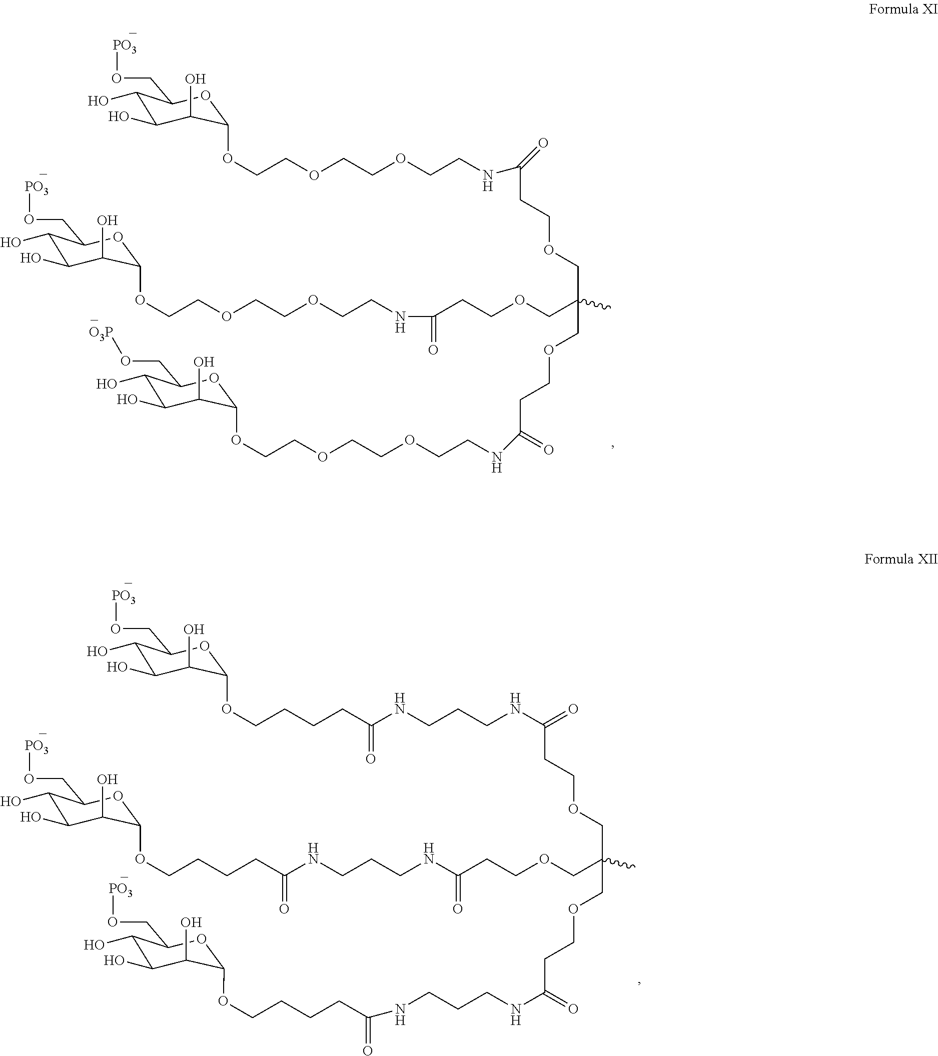

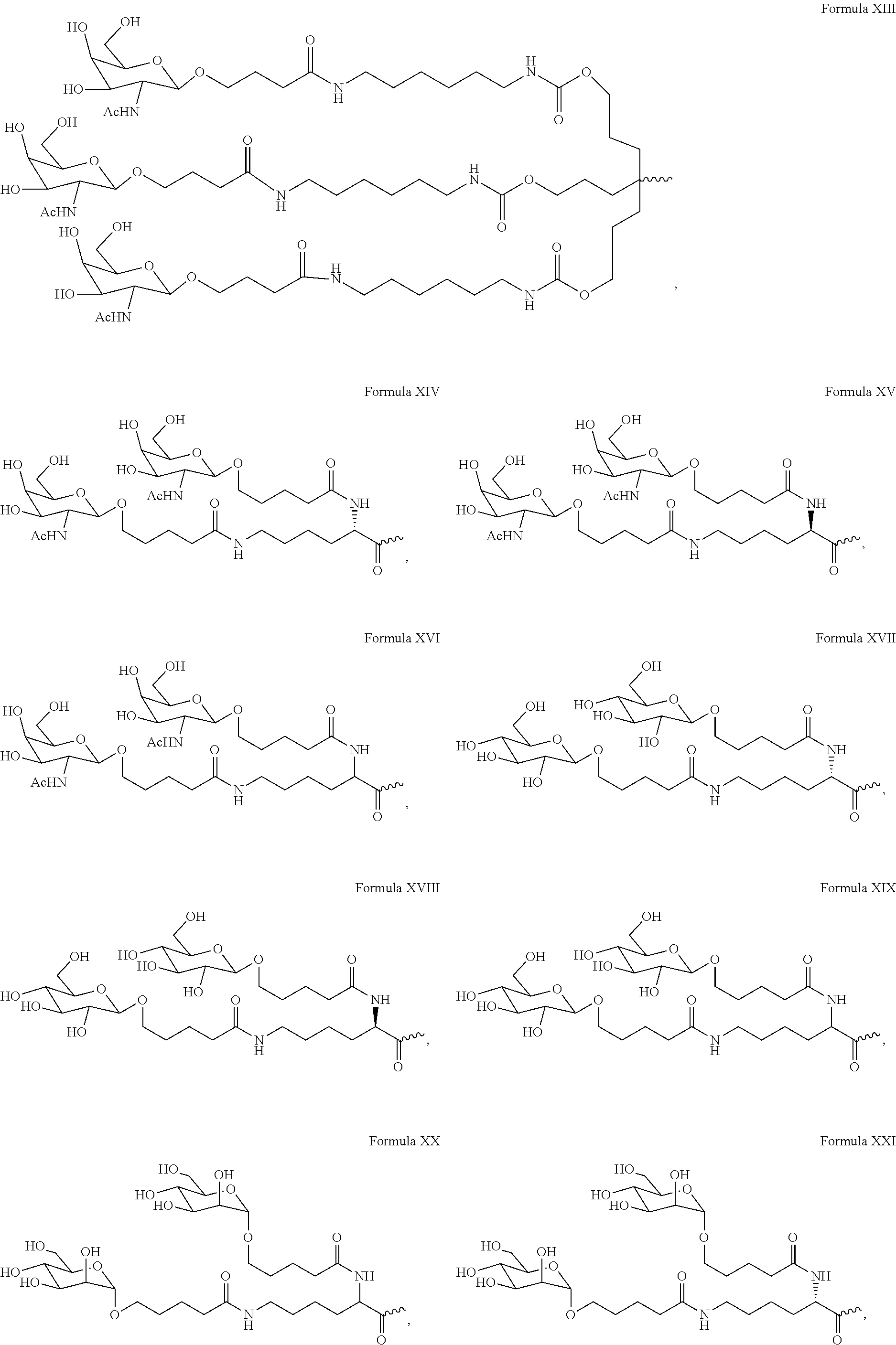

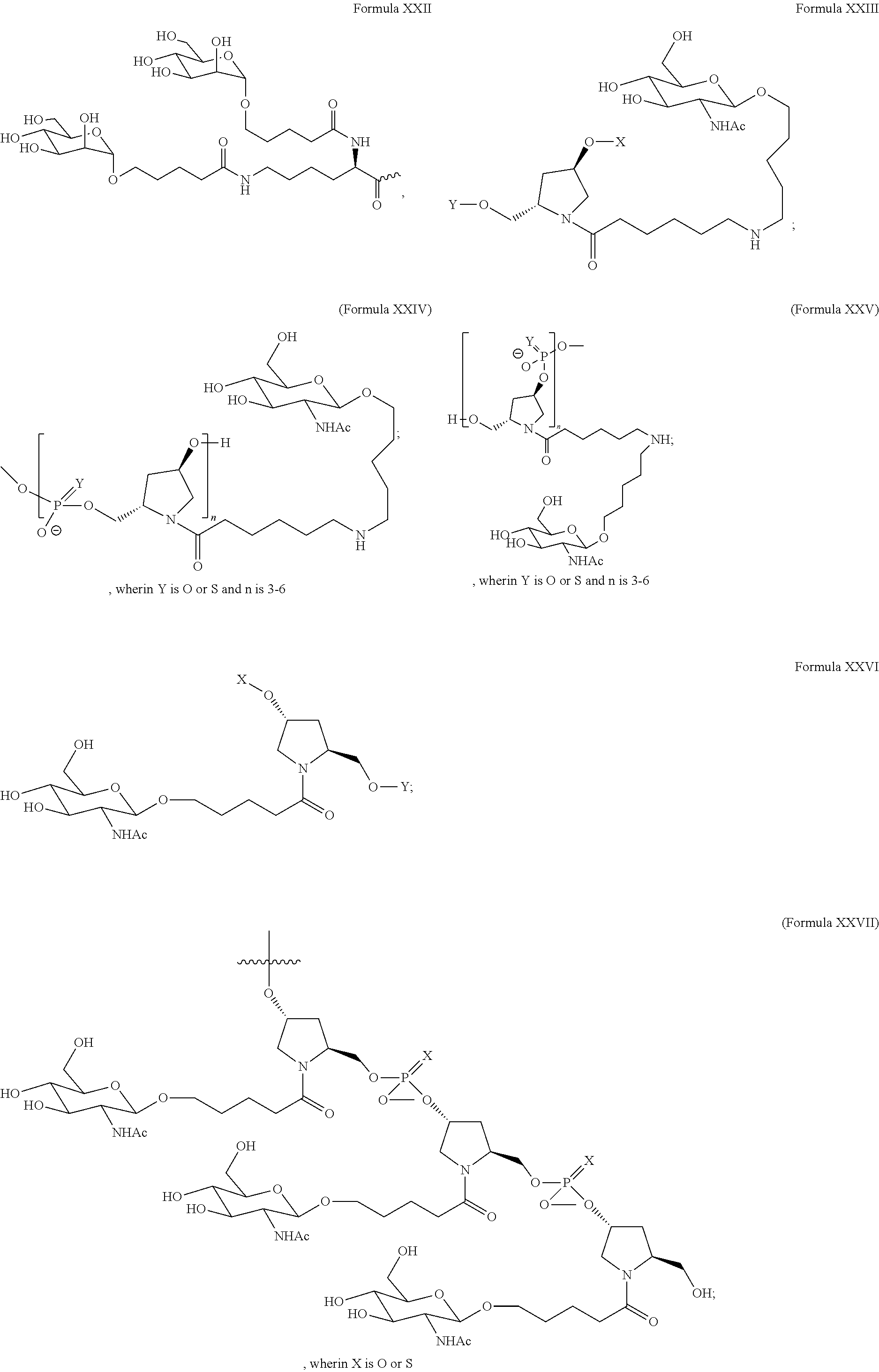

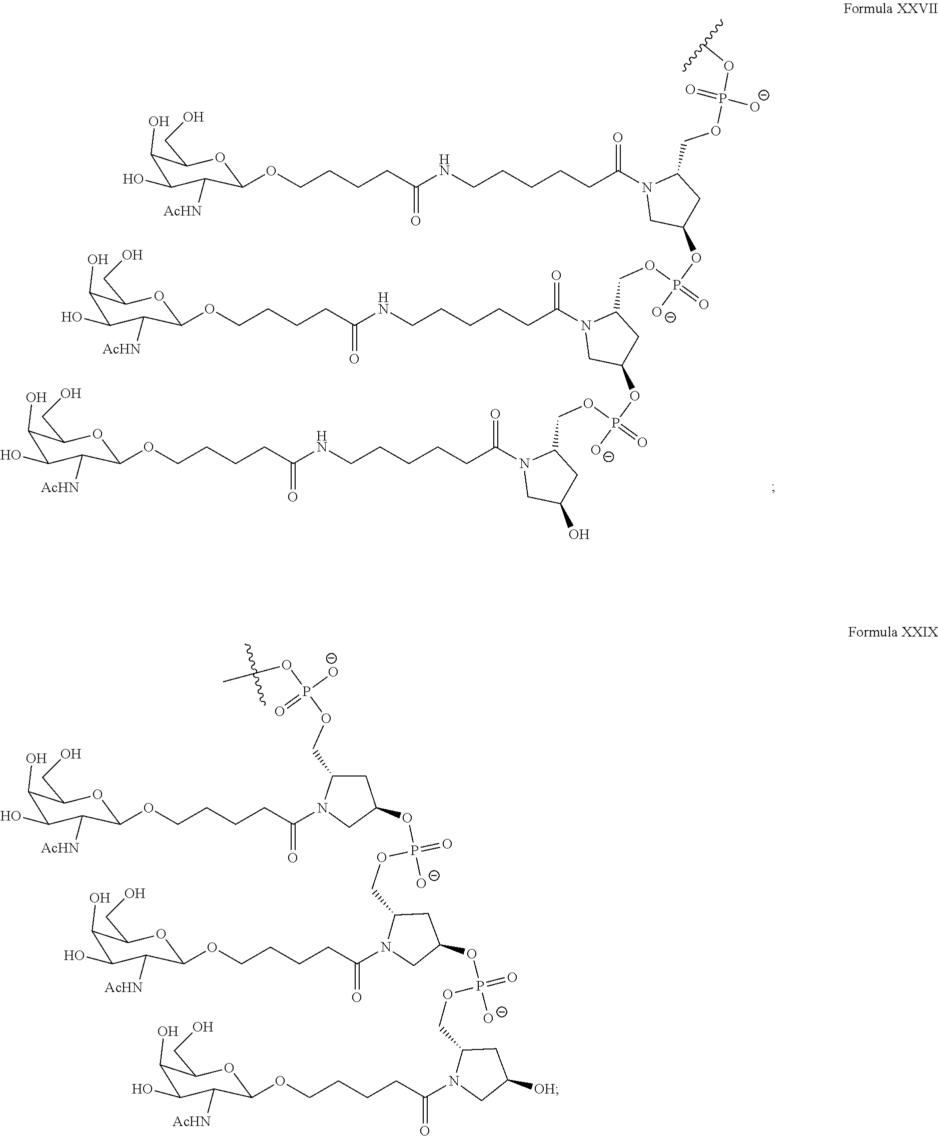

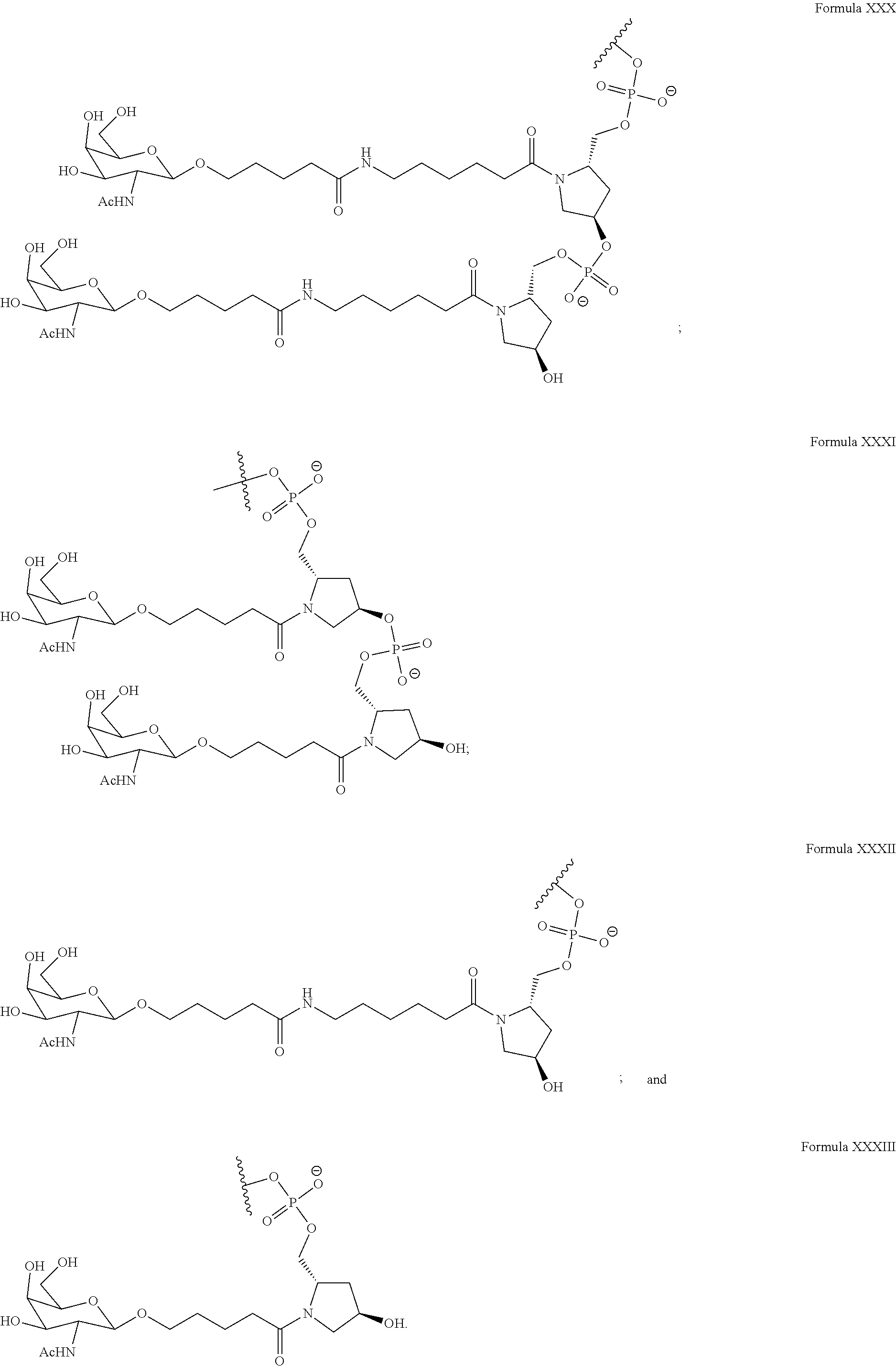

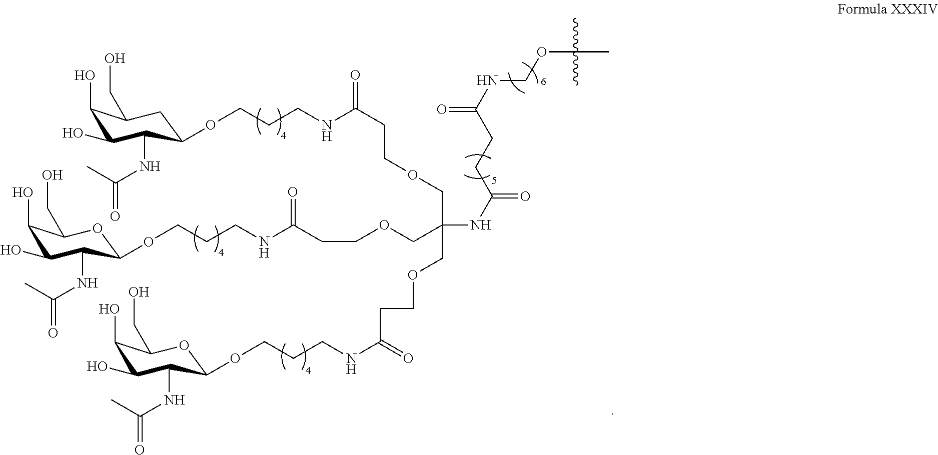

In another embodiment, a carbohydrate conjugate for use in the compositions and methods of the invention is a monosaccharide. In one embodiment, the monosaccharide is an N-acetylgalactosamine, such as

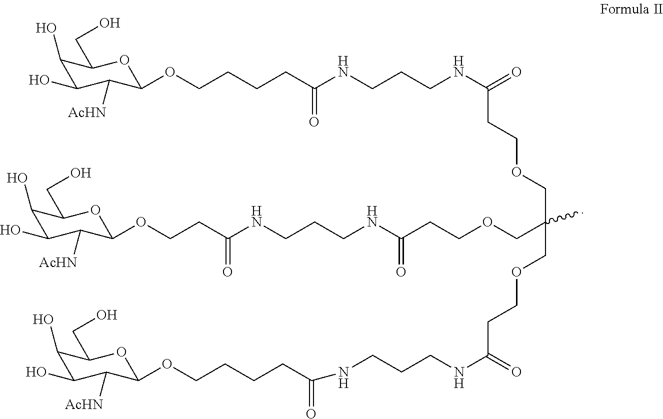 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCOC2OC(CO)C(O)C(O)C2NC(C)=O)OC(CO)C(O)C1O]

Another representative carbohydrate conjugate for use in the embodiments described herein includes, but is not limited to,

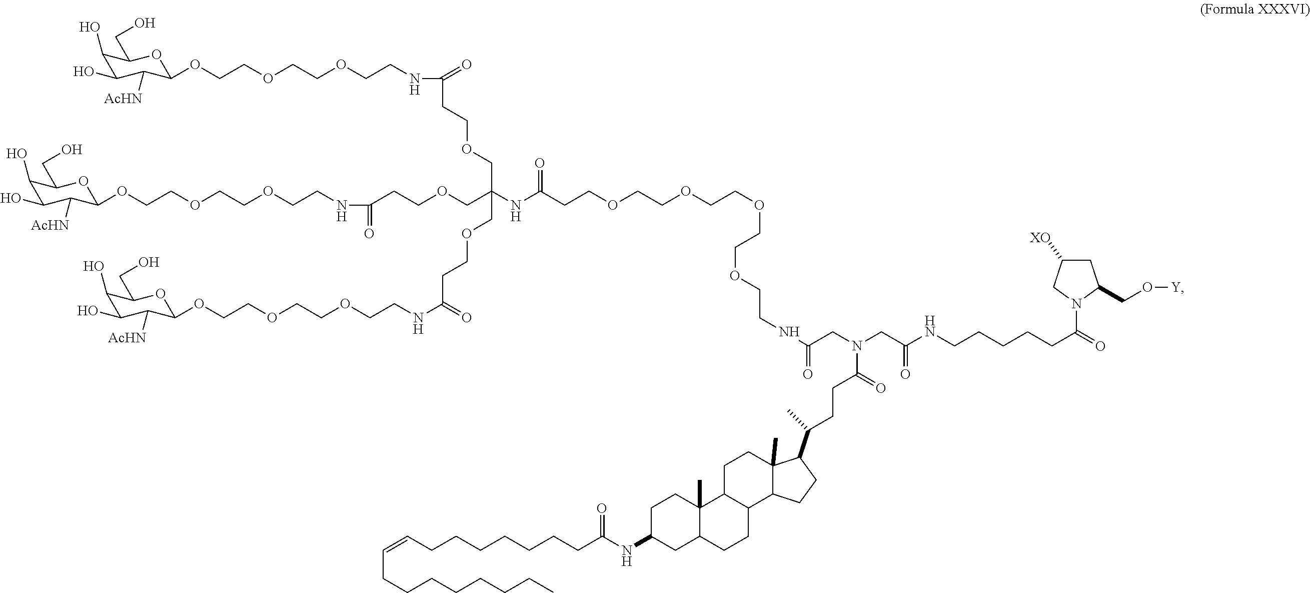

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

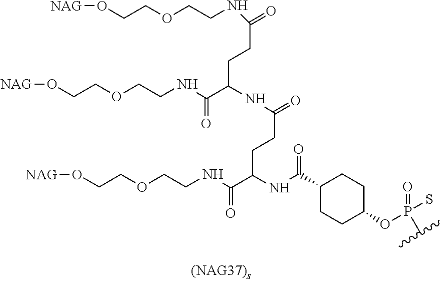

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one GalNAc or GalNAc derivative attached to the iRNA agent, e.g., the 5′ end of the sense strand of a dsRNA agent, or the 5′ end of one or both sense strands of a dual targeting RNAi agent as described herein. In another embodiment, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in PCT Publication Nos. WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In other embodiments, a cleavable linker comprises a phosphate-based cleavable linking group.

A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. A preferred embodiment is —O—P(O)(OH)—O—. These candidates can be evaluated using methods analogous to those described above.

iii. Acid Cleavable Linking Groups

In other embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In other embodiments, a cleavable linker comprises an ester-based cleavable linking group. An ester-based cleavable linking group is cleaved by enzymes such as esterases and amidases in cells. Examples of ester-based cleavable linking groups include, but are not limited to, esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet other embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of the compositions and methods of the invention include, but are not limited to,

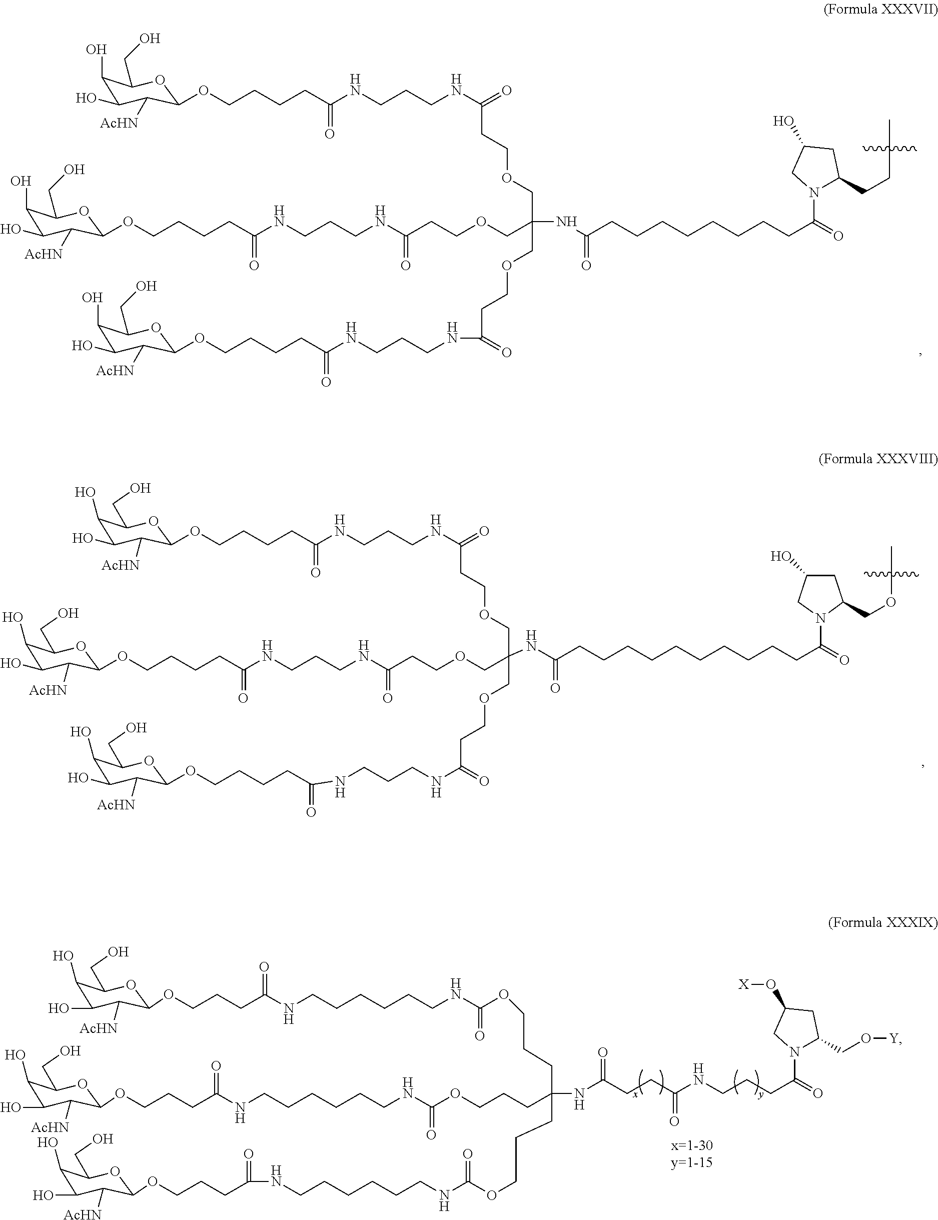

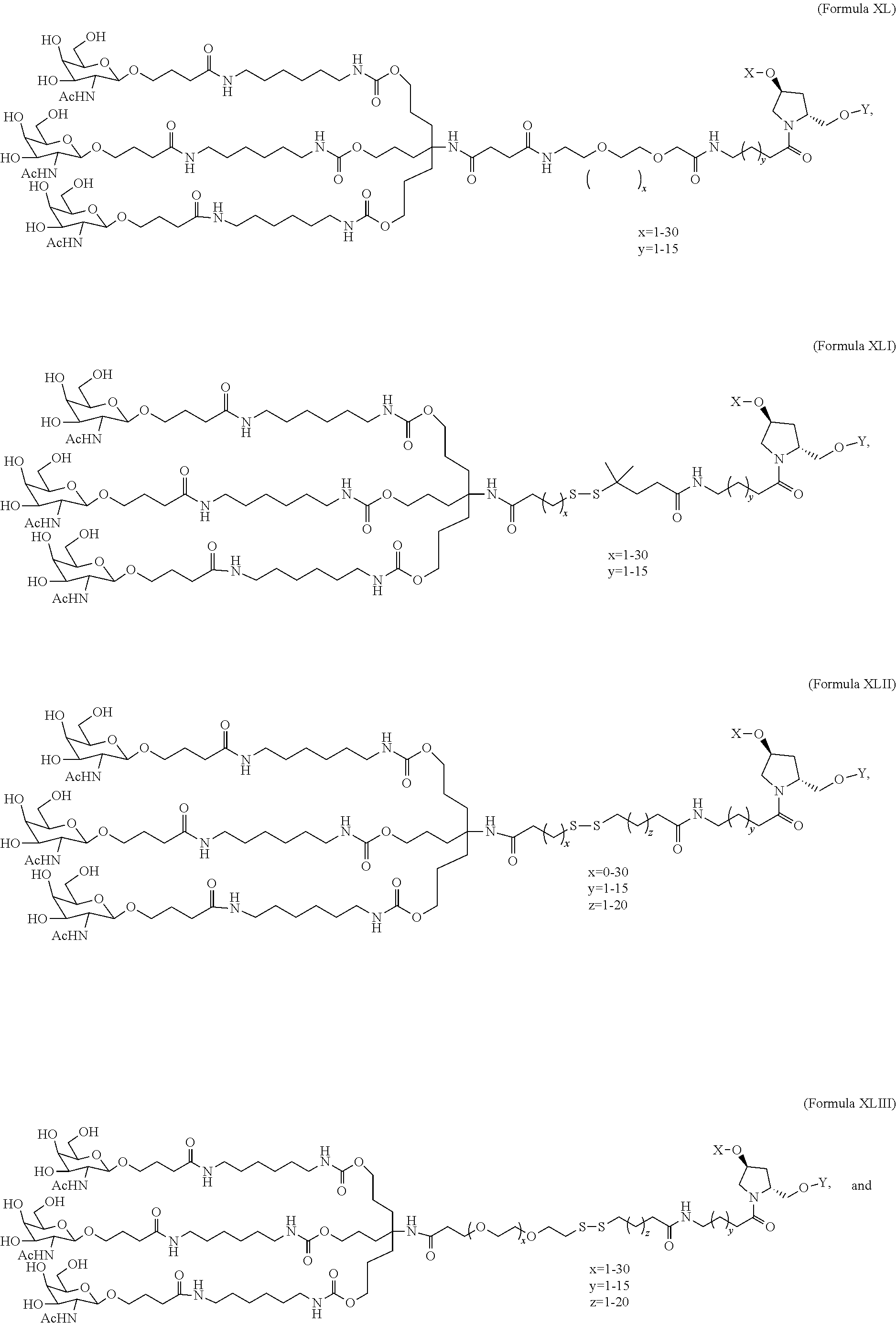

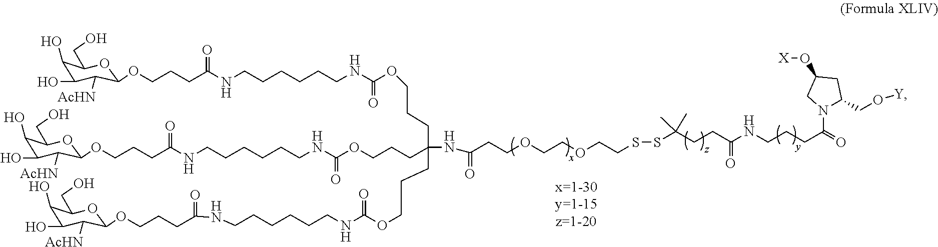

when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XLV)-(XLVI):

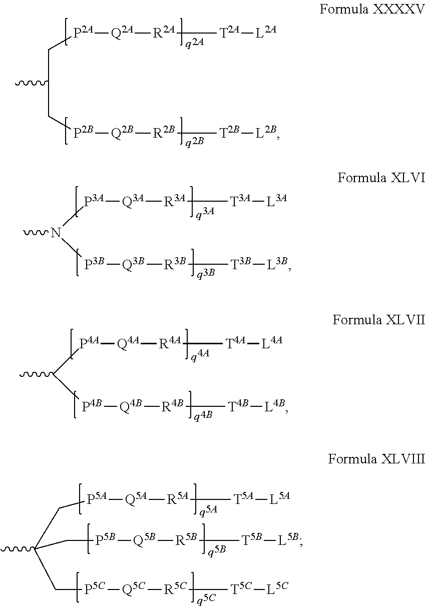

wherein:
q2A, q2B, q3A, q3B, q4A, q4B, q5A, q5B and q5C represent independently for each occurrence 0-20 and wherein the repeating unit can be the same or different;
P2A, P2B, P3A, P3B, P4A, P4B, P5A, P5B, P5C, T²A, T²B, T³A, T³B, T⁴A, T⁴B, T⁴A, T⁵B, T^{5C} are each independently for each occurrence absent, CO, NH, O, S, OC(O), NHC(O), CH₂, CH₂NH or CH₂O;
Q²A, Q²B, Q³A, Q³B, Q⁴A, Q⁴B, Q⁵A, Q⁵B, Q^{5C} are independently for each occurrence absent, alkylene, substituted alkylene wherein one or more methylenes can be interrupted or terminated by one or more of 0, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

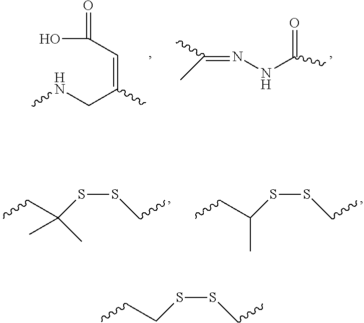

or heterocyclyl; L²A, L²B, L³A, L³B, L⁴A, L^{4B} L⁵A, L^{5B} and L⁵c represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

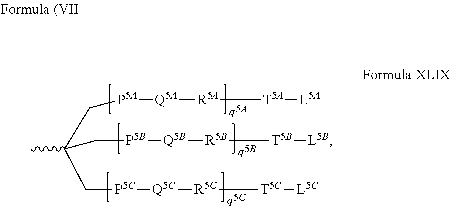

wherein L⁵A, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928;5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### VIII. Pharmaceutical Compositions of the Invention

The present invention also includes pharmaceutical compositions and formulations which include the iRNAs for use in the methods of the invention. In one embodiment, provided herein are pharmaceutical compositions containing an iRNA, as described herein, and a pharmaceutically acceptable carrier. The pharmaceutical compositions containing the iRNA are useful for preventing or treating an AGT associated disorder, e.g., hypertension. Such pharmaceutical compositions are formulated based on the mode of delivery.

The pharmaceutical compositions comprising RNAi agents of the invention may be, for example, solutions with or without a buffer, or compositions containing pharmaceutically acceptable carriers. Such compositions include, for example, aqueous or crystalline compositions, liposomal formulations, micellar formulations, emulsions, and gene therapy vectors.

In the methods of the invention, the RNAi agent may be administered in a solution. A free RNAi agent may be administered in an unbuffered solution, e.g., in saline or in water. Alternatively, the free siRNA may also be administered in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject.

In some embodiments, the buffer solution further comprises an agent for controlling the osmolarity of the solution, such that the osmolarity is kept at a desired value, e.g., at the physiologic values of the human plasma. Solutes which can be added to the buffer solution to control the osmolarity include, but are not limited to, proteins, peptides, amino acids, non-metabolized polymers, vitamins, ions, sugars, metabolites, organic acids, lipids, or salts. In some embodiments, the agent for controlling the osmolarity of the solution is a salt. In certain embodiments, the agent for controlling the osmolarity of the solution is sodium chloride or potassium chloride.

In some embodiments, the pharmaceutical compositions of the invention are pyrogen free or non-pyrogenic.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

One example is compositions that are formulated for systemic administration via parenteral delivery, e.g., by subcutaneous (SC), intramuscular (IM), or intravenous (IV) delivery. The pharmaceutical compositions of the invention may be administered in dosages sufficient to inhibit expression of an AGT gene.

The pharmaceutical compositions of the invention may be administered in dosages sufficient to inhibit expression of an AGT gene. In some embodiments, a fixed dose of about 50 mg to about 800 mg (e.g., about 50 to about 200 mg, about 50 mg to about 500 mg, about 100 mg to about 800 mg, about 100 mg to about 500 mg, about 100 mg to about 300 mg, about 200 mg to about 300 mg, about 200 mg to about 400 mg, about 200 mg to about 500 mg, about 200 mg to about 800 mg, about 300 mg to about 800 mg, about 300 mg to about 500 mg, about 300 mg to about 4000 mg, about 400 mg to about 800 mg, about 400 mg to about 500 mg, e.g., about 50, 100, 150, 200, 250, 300, 350, 400, 450, 500, 550, 600, 650, 700, 750, or about 800 mg) of the iRNA agents are administered to the subject.

A repeat-dose regimen may include administration of a therapeutic amount of iRNA on a regular basis, such as every month, every two months, every three months, every four months, every five months, every six months, once every 3-6 months, or once a year. In certain embodiments, the iRNA is administered about once per month to about once per quarter to about once per six months.

After an initial treatment regimen, the treatments can be administered on a less frequent basis. Duration of treatment can be determined based on the severity of disease.

In other embodiments, a single dose of the pharmaceutical compositions can be long lasting, such that doses are administered at not more than 1, 2, 3, 4, 5 or 6 month intervals. In some embodiments of the invention, a single dose of the pharmaceutical compositions of the invention is administered about once per month. In other embodiments of the invention, a single dose of the pharmaceutical compositions of the invention is administered quarterly (i.e., about every three months). In other embodiments of the invention, a single dose of the pharmaceutical compositions of the invention is administered twice per year (i.e., about once every six months).

The skilled artisan will appreciate that certain factors can influence the dosage and timing required to effectively treat a subject, including but not limited to mutations present in the subject, previous treatments, the general health or age of the subject, and other diseases present. Moreover, treatment of a subject with a prophylactically or therapeutically effective amount, as appropriate, of a composition can include a single treatment or a series of treatments.

The iRNA can be delivered in a manner to target a particular tissue (e.g., hepatocytes).

Pharmaceutical compositions of the present invention include, but are not limited to, solutions, emulsions, and liposome-containing formulations. These compositions can be generated from a variety of components that include, but are not limited to, preformed liquids, self-emulsifying solids, and self-emulsifying semisolids. Formulations include those that target the liver.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers.

### IX. Kits

The present invention also provides kits for performing any of the methods of the invention. Such kits include one or more double stranded RNAi agent(s) and instructions for use, e.g., instructions for administering a fixed dose of a double stranded RNAi agent(s). The double stranded RNAi agent may be in a vial or a pre-filled syringe. The kits may optionally further comprise means for administering the double stranded RNAi agent (e.g., an injection device, such as a pre-filled syringe), or means for measuring the inhibition of AGT (e.g., means for measuring the inhibition of AGT mRNA, AGT protein, and/or AGT activity). Such means for measuring the inhibition of AGT may comprise a means for obtaining a sample from a subject, such as, e.g., a plasma sample. The kits of the invention may optionally further comprise means for determining the therapeutically effective or prophylactically effective amount.

Unless otherwise defined, all technical and scientific terms used herein have the same meaning as commonly understood by one of ordinary skill in the art to which this invention belongs. Although methods and materials similar or equivalent to those described herein can be used in the practice or testing of the iRNAs and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

This invention is further illustrated by the following examples which should not be construed as limiting. The entire contents of all references, patents and published patent applications cited throughout this application, as well as the Sequence Listing, are hereby incorporated herein by reference.

### EXAMPLES

Abbreviations of nucleotide monomers used in nucleic acid sequence representation. It will be understood that these monomers, when present in an oligonucleotide, are mutually linked by 5′-3′-phosphodiester bonds unless otherwise indicated.

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine -3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (Hyp-(GalNAc-alkyl)3) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |

### Example 1. Phase 1 Clinical Trial of AGT dsRNA

A multicenter, randomized, double-blind, active comparator single dose and multiple dose clinical trial was designed to evaluate the safety, tolerability, pharmacokinetic (PK), and pharmacodynamic (PD) effects of subcutaneous administration of (SC) AD-85481 to patients with hypertension.

This study includes 4 parts:
Part A: single ascending dose (SAD) phase in hypertensive patients;
Part B: Single dose (SD) in hypertensive patients with controlled salt intake;
Part C: Multiple dose (MD) phase in hypertensive patients; and
Part D: Multiple dose (MD) phase in hypertensive patients who are obese.

Ongoing reviews of safety, tolerability, and available PD and PK data were collected in all parts of the study.

### Diagnosis and Main Eligibility Criteria

This study included adults 18 to 65 years of age with hypertension (mean sitting systolic blood pressure [SBP] of >130 and ≤159 mmHg). Patients with secondary hypertension or mean sitting diastolic blood pressure [DBP]≥100 mmHg were excluded. Patients with clinically significant medical conditions or comorbidities that would interfere with study compliance or data interpretation (including diabetes mellitus or history of any cardiovascular event) or who were currently taking or anticipated using medications known to affect blood pressure were also excluded.

### Study Drug, Dose, and Mode of Administration

Study drug, AD-85481, a chemically modified, N-acetylgalactosamine (GalNAc)-conjugated small interfering RNA (siRNA) designed to suppress production of angiotensinogen (AGT) as a strategy to lower blood pressure in individuals with hypertension, was administered either as a single subcutaneous injection (Parts A and B) or 2 subcutaneous injections administered once at Week 1 and once at Week 12 (Parts C and D).

Unmodified and Modified Nucleotide Sequence of AD-85481

| | | SEQ | | SEQ |
|---|---|---|---|---|
| Strand | Unmodified (5′ to 3′) | ID | Modified (5′ to 3′) | ID |
| Antis | UGUACUCUCAUUGUGGAUGACGA | 9 | usGfsuac(Tgn)cucauugUfgGfaugacsgsa | 11 |
| Sense | GUCAUCCACAAUGAGAGUACA | 10 | gsuscaucCfaCfAfAfugagaguaca | 12 |

The chemical modifications are defined as follows: a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Ggn) is guanosine-glycol nucleic acid (GNA), and s is phosphorothioate linkage. The 3′end of the sense strand is covalently linked to an N-[tris(GalNAc-alkyl)-amidodecanoyl)]-4-hydroxyprolinol (also referred to as Hyp-(GalNAc-alkyl)3 or L96) ligand.

### Placebo Control, Dose, and Mode of Administration

In Parts A and B, the control drug for AD-85481 was a placebo (normal saline 0.9% for subcutaneous administration).

In Parts C and D, the dummy treatment for AD-85481 is normal saline 0.9% for subcutaneous administration. The dummy treatment for irbesartan is an inert dummy tablet that matches the appearance of irbesartan.

### Active Comparator Control, Dose, and Mode of Administration

In Parts C and D of this study, once daily oral (PO) doses of 150 mg irbesartan is used as the active comparator.

### Duration of Treatment and Study Participation

A single subcutaneous dose of AD-85481 was administered in Parts A and B. In Parts C and D, 2 subcutaneous doses of study drug are administered over 12 weeks.

### Statistical Methods

The sample size was based on practical considerations and is consistent with this type of early phase study.

The Full Analysis Set (FAS) included all patients who received any amount of study drug grouped according to the treatment to which they were randomized. The PK Analysis Set included all patients who received at least 1 dose of study drug and have at least 1 post-dose blood sample for determination of AD-85481 concentrations and have evaluable PK data. The PD Analysis Set included all patients who received at least 1 dose of study drug and who had at least 1 post-dose blood sample for the determination of serum AGT.

AD-85481 activity was analyzed using the FAS. The PK and PD Analysis Sets were used to conduct PK and PD analyses, respectively.

Statistical analyses are primarily descriptive in nature. Descriptive statistics (e.g., mean, standard deviation, median, minimum, and maximum) are presented for continuous variables. Frequencies and percentages are presented for categorical and ordinal variables. Descriptive statistics are provided for clinical laboratory data, electrocardiogram (ECG), and vital signs data.

### Study Design Rationale

A Phase 1, multi-center, randomized, double-blind study of AD-85481 was administered subcutaneously (SC) to patients with hypertension. The primary objective for the study was to evaluate the safety and tolerability of single or multiple doses of AD-85481 in patients with hypertension. The study was conducted in 4 parts: a single ascending dose (SAD) phase (Part A), a single dose (SD) phase in patients with controlled salt intake (Part B), a multiple dose (MD) phase (Part C), and a MD phase in obese patients (Part D).

The study was designed to provide initial insight into the safety and tolerability of AD-85481. Based upon the known safety profile of approved antihypertensives, this study was designed to carefully monitor blood pressure, serum electrolytes, and creatinine, with frequent data collection during the anticipated nadir of AGT (estimated to be 4-6 weeks after AD-85481 dose). Clinical laboratory assessments and blood pressure data were collected periodically after study drug administration to correspond with the AGT nadir.

In addition to safety of AD-85481, this study was designed to enable insights into the following secondary or exploratory research questions.

Pharmacodynamic (PD) of AD-85481: PD effect was directly demonstrated by serial measurements of serum AGT to determine the change from baseline in the level of plasma AGT. Downstream effectors of AGT (plasma renin concentration, plasma renin activity, angiotensin I, angiotensin II, aldosterone) may also be measured in blood and urine specimens as exploratory biomarkers.

Pharmacokinentic (PK) of AD-85481: PK parameters of AD-85481 were assessed by determination of plasma and urine levels of AD-85481 and potential metabolites, e.g., by qPCR.

Blood pressure reduction: This study enrolled patients with hypertension (initially corresponding to Grade 1 per ESC/ESH criteria) to enable the assessment of therapeutic blood pressure reduction. Single dose phases (Parts A and B) are of short duration and use an inert placebo group. To adhere to best ethical standards, longer MD phases (Parts C and D) use an established angiotensin II-receptor blocker (ARB) (irbesartan) as an active comparator to AD-85481 treatment. Exploratory assessments of AD-85481 include change from baseline in SBP and DBP assessed by 24 hour ambulatory blood pressure monitoring (ABPM) and the change from baseline in SBP and DBP assessed by oscillometric automated office blood pressure (AOBP) and by oscillometric home blood pressure monitoring (HBPM).

As recommended by current guidance, patients discontinue prior antihypertensive medications for approximately 3-4 weeks prior to study drug administration and blood pressure was monitored with both automated office blood pressure (AOBP) measurements and by outpatient 24-hour ambulatory blood pressure monitoring (ABPM). In addition to having greater precision, the latter method assesses peak-to-trough blood pressure ratios and circadian rhythms (including potential restoration of the normal nocturnal blood pressure dipping pattern that is lost in 24-35% of hypertensive patients). More frequent (daily) measurements are collected through a third method, oscillometric home blood pressure monitoring (HBPM), to characterize gradual PD effects and to provide close safety monitoring for potential hypotension while not in the clinic.

Impact of Low Salt Intake on Blood Pressure Response to AD-85481: Because hypertension is salt-sensitive in some patients, all study patients receive educational materials on diet with instructions to limit sodium consumption to approximately 2.0 g per day from screening through end of treatment (EOT). Of note, this is the sodium intake recommended in the 2018 ESC/ESH Guidelines for both hypertensive patients and for the general population.

Additionally, the Part B study 1 cohort of patients with controlled salt intake are studied to evaluate potential for augmented pharmacology and safety in a low-salt state (1.15 g sodium per day), which has been demonstrated previously with other RAAS-inhibiting drugs. Part B patients complete a 2-week dietary subprotocol that varies sodium consumption to directly test for salt-sensitive blood pressure responses. During Part B, food is prepared in research/metabolic kitchens according to a common protocol and provided to patients. Patients are inpatient for the first week of the 2-week subprotocol to promote compliance with a low-salt diet and to enhance monitoring for potential augmented AD-85481 pharmacology upon the introduction of salt deprivation. After AGT lowering and salt deprivation, the blood pressure response to high salt intake alone is characterized. Exploratory endpoints on the effects of AD-85481 include determination of change from baseline in SBP and DBP assessed by ABPM and AOBP under low vs high salt conditions

Obesity: The majority of patients with hypertension are expected to be overweight or obese. Additionally, increased adiposity is likely to be causally associated with hypertension. Part D studies 1 cohort of patients with body mass index (BMI) in the Class I to III obesity range to evaluate the impact of weight on PK and PD parameters.

As preclinical studies have implicated AGT as a contributor to both diet-induced obesity and hepatic steatosis via mechanisms that may be distinct from classic RAAS pathways, weight loss is evaluated as an exploratory endpoint. Anthropometrics (waist circumference, waist-to-hip ratio) and a radiographic assessment of body composition (dual-energy x-ray absorptiometry; DEXA) are collected to determine if weight changes are due to loss of body fat. Biochemical parameters of lipid and glucose metabolism (lipid profile, HbA1c, fasting glucose) that improve with weight reduction are measured approximately every 3 months. Of note, Parts A, B, and C enroll patients in the normal to overweight, and mild (Class I) obesity range (BMI≥18 kg/m² and ≤35 kg/m²). The single cohort in Part D is restricted to obese patients (BMI>30 kg/m² and ≤50 kg/m²). Exploratory endpoints on the effects of AD-85481 include determination of change from baseline in body weight, waist circumference, waist-to-hip ratio, and body composition (as assessed by dual-energy x-ray absorptiometry (DEXA)) in obese patients.

Further exploratory objectives of the study overall include assessment of the effect of ALN-AD-85481 on metabolic syndrome parameters by determining the change from baseline in HbA1c, fasting plasma glucose, and serum lipid profile; and assessment of the effect of AD-85481 on exploratory biomarkers of the RAAS by determining the change from baseline in plasma renin concentration, plasma renin activity, angiotensin I, angiotensin II, and aldosterone.

### Study Drug Dosing and Progression.

Doses for Part A are 10 mg, 25 mg, 50 mg, 100 mg, 200 mg, and ≤400 mg. There were 12 patients in each of the 6 cohorts (with 3 optional cohorts for evaluation of interim dose levels, lower dose levels, or expansions of previous cohorts may be enrolled in Part A to better characterize the dose response or safety and tolerability) with a 2:1 randomization of AD-85481:placebo. The lowest dose is not expected to have an effect on blood pressure lowering. Blood pressure lowering activity was expected to be first observed at the 50 mg dose with a lowering of serum AGT of at least 80%.

### Phase I—Part A

Lowering of serum AGT from baseline in Part A. Subjects meeting inclusion and exclusion criteria were administered a single dose of AD-85481 or placebo on Day 1. Blood samples were collected prior to administration of AD-85481 or placebo, weekly for 6 weeks after administration, at week 8 and week 12 post treatment, and then every 3 months during the follow-up period. Serum AGT level was determined by solid phase sandwich ELISA and lowering of AGT from baseline was determined at each of the time points.

A total of 60 patients with hypertension completed treatment in Part A of the study. Patients received either placebo (n=4 per cohort) or AD-85481 (n=8 per cohort). The demographics and baseline characteristics of subjects participating in Part A are presented in the table below.

| | | Placebo | 10 mg | 25 mg | 50 mg | 100 mg | 200 mg |
|---|---|---|---|---|---|---|---|
| | | (N = 20) | (N = 8) | (N = 8) | (N = 8) | (N = 8) | (N = 8) |
| Age, years; median | | 52 | 53 | 56 | 41 | 56 | 56 |
| (range) | | (36-64) | (37-60) | (47-63) | (35-64) | (35-65) | (43-64) |
| Gender | Male | 9 | 7 | 2 | 7 | 3 | 5 |
| | Female | 11 | 1 | 6 | 1 | 5 | 3 |
| Race | White | 14 | 6 | 4 | 3 | 4 | 6 |
| | Black | 5 | 1 | 4 | 4 | 2 | 2 |
| | Asian | 0 | 1 | 0 | 0 | 2 | 0 |
| | Other | 1 | 0 | 0 | 1 | 0 | 0 |
| Blood | 24 h ABPM | 141 | 139 | 141 | 135 | 136 | 139 |
| | SBP | (126,153) | (130, 147) | (132, 157) | (113, 144) | (131, 152) | (129, 154) |
| | median | | | | | | |
| | (range) | | | | | | |
| Pressure | 24 h ABPM | 87 | 84 | 91 | 84 | 86 | 83 |
| | DBP | (72-102) | (76, 93) | (75, 103) | (74, 91) | (80, 90) | (75, 95) |
| | median | | | | | | |
| | (range) | | | | | | |

Safety profile in Part A. The primary objective for the study was to evaluate the safety and tolerability of single doses of AD-85481 in patients with hypertension. As demonstrated in the table below, the safety profile of AD-85481 was acceptable with no safety concerns. Most adverse events were mild or moderate in severity and were resolved without intervention. There was neither death or adverse events leading to study withdrawal, nor treatment-related serious adverse events (SAEs). Severe SAE of prostate cancer was reported in 1 patient who received 200 mg, based upon a biopsy that was performed in the screening period and was reported as positive after dosing. No patient required intervention for low blood pressure, and no clinically significant elevations in serum alanine aminotransferase (ALT), serum creatinine, or serum potassium were observed during the course of the study. Five patients reported mild injection site reactions

| Patients Reporting | | | | | | |
|---|---|---|---|---|---|---|
| an Adverse | Placebo | 10 mg | 25 mg | 50 mg | 100 mg | 200 mg |
| Event (AE), N | (N = 20) | (N = 8) | (N = 8) | (N = 8) | (N = 8) | (N = 8) |
| At least 1 adverse | 17 | 5 | 7 | 6 | 7 | 7 |
| event | | | | | | |
| At least 1 serious | 1 | 0 | 0 | 0 | 0 | 1 |
| adverse event | | | | | | |
| At least 1 severe | 1 | 0 | 0 | 0 | 0 | 1 |
| adverse event | | | | | | |

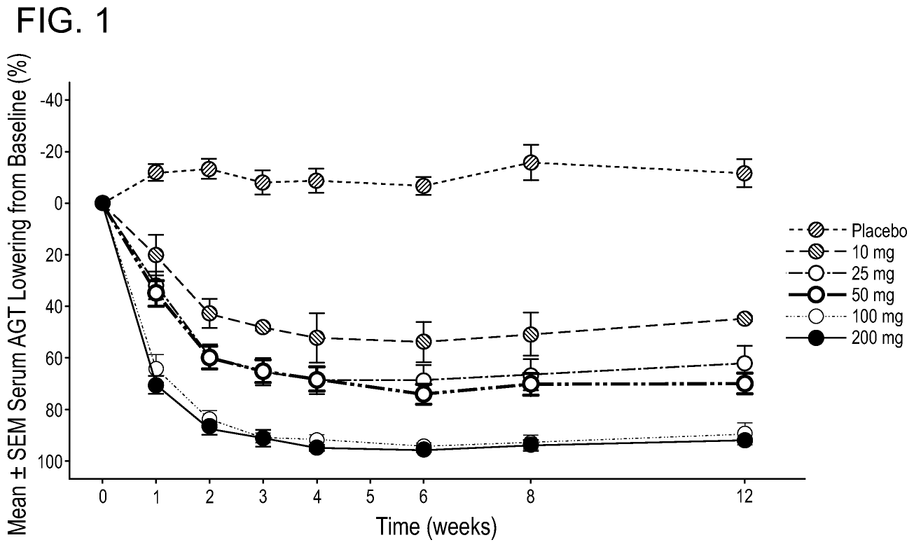

The effects of administration of a single dose of AD-85481 on the level of serum AGT are presented in FIG. 1.

A mean maximum AGT lowering of 54% was observed at 10 mg dose with a mean lowering at 4 weeks of 52% at the 10 mg single dose.

A mean maximum AGT lowering of 69% was observed at the 25 mg dose with a mean lowering at 4 weeks of 69% at the 25 mg single dose.

A mean maximum AGT lowering of 74% was observed at the 50 mg dose with a mean lowering at 4 weeks of 68% at the 50 mg single dose.

A mean maximum AGT lowering of 94% was observed at the 100 mg dose with a mean lowering at 4 weeks of 92% at the 100 mg single dose.

A mean maximum AGT lowering of 96% was observed at the 200 mg dose with a mean lowering at 4 weeks of 95% at the 200 mg single dose.

These data demonstrate a durable dose-dependent reduction of serum AGT after treatment with a single dose of AD-85481. In addition, a greater than 90% reduction in serum AGT levels was observed in subjects receiving higher single doses of AD-85481, and the reduction in AGT persisted for greater than 3 months, e.g., demonstrating that a chronic dosing administration interval of at least once quarterly would be effective.

Modulation of blood pressure from baseline in Part A. Assessments of AD-85481 included change from baseline in SBP and DBP assessed by 24 hour ambulatory blood pressure monitoring (ABPM) and the change from baseline in SBP and DBP assessed by oscillometric automated office blood pressure (AOBP) and by oscillometric home blood pressure monitoring (HBPM).

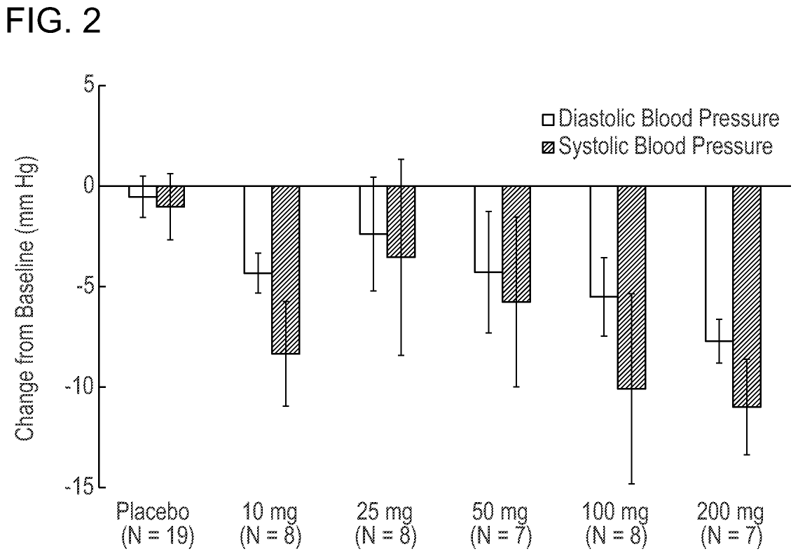

As depicted in FIG. 2, a single 100 mg dose of AD-85481, reduced systolic and diastolic blood pressures by about 10.1 mmHg and about 5.5 mmHg, respectively, at Week 8 when compared to placebo as determined by 24 hour ambulatory blood pressure measurements (ABPM). After a single 200 mg dose of AD-85481, reductions in systolic and diastolic blood pressures of about 11 mmHg and about 7.7 mmHg, respectively, at Week 8 compared to placebo were observed, as determined by 24 hour ambulatory blood pressure measurements (ABPM).

These data demonstrate a dose-dependent reduction in SBP and DBP in subjects receiving single doses of AD-85481. Specifically, greater than 10 mmHg reduction in 24 hour SBP was observed at Week 8 after a single dose of AD-85481 (e.g., 100 mg or 200 mg).

### SUMMARY

In summary, this single ascending dose study characterized the maximum effect of AD-85481 and demonstrated the durability of AD-85481 treatment for a period of over 3 months. These data demonstrate that single subcutaneous doses of AD-85481 (also referred to ALN-AGT01) were well tolerated in patients with mild to moderate hypertension, with no treatment-related serious adverse events. Administration of AD-85481 led to a dose-dependent and durable reduction of serum AGT. AGT reductions of greater than 90% were observed after higher single doses of AD-85481 that persisted for ≥3 months, demonstrating that an infrequent dosing interval is required.

Blood pressure reductions mirrored AGT knockdown following administration of a single fixed dose of AD-85481, with >10 mm Hg reduction in 24 hour SBP observed at 8 weeks after single doses of 100 mg or higher.

As compared to current methods and therapeutics to treat hypertension, many of which are associated with negative effects on kidney function, these data further demonstrate that liver-specific silencing of AGT is effective and, thus, provide improved renal safety. The prolonged duration of action of AD-85481 is further superior to current therapeutics in that it may provide a consistent and durable blood pressure response, blunting of diurnal BP variation, and enhanced adherence since infrequent dosing is required and there is a reduction in overall pill burden.

### Example 2. Phase II Clinical Trial of AGT dsRNA

A randomized, double-blind, placebo-controlled, dose-ranging, multicenter study was designed to evaluate the safety, efficacy, and pharmacodynamics (PD) of ALN-AGT01, administered subcutaneously (SC), in patients with mild-to-moderate hypertension. Before randomization, patients will discontinue prior antihypertensive medications (if taking) for a Washout period of at least 4 weeks. Patients who complete the Screening period and meet all inclusion/exclusion criteria for enrollment will receive ALN-AGT01 or placebo for the first 6 months of the 12-month Double-blind (DB) treatment period.

### Diagnosis and Main Eligibility Criteria

This study includes adults (18 to 75 years, inclusive) with untreated hypertension or on stable therapy with 1 or more antihypertensive medications of the following classes: an angiotensin converting enzyme inhibitor, angiotensin II-receptor blocker, renin inhibitor, calcium channel blocker, thiazide diuretic, and/or thiazide-like diuretic. Patients should have a daytime mean systolic blood pressure (SBP)≥135 mmHg and ≤160 mmHg by ABPM at least 4 weeks after washout of background antihypertensive medication. Patients with secondary hypertension or orthostatic hypotension will be excluded.

### Study Drug, Dose, and Mode of Administration

ALN-AGT01, also known as AD-85481, is a subcutaneously administered N-acetylgalactosamine-conjugated small interfering RNA targeting liver-expressed messenger RNA for angiotensinogen (AGT). The unmodified and modified nucleotide sequences of ALN-AGT01 are provided above.

Patients randomized to receive ALN-AGT01 will be administered 150 mg ALN-AGT01 SC once every 6 months, 300 mg ALN-AGT01 SC once every 6 months, 300 mg ALN-AGT01 SC once every 3 months, or 600 mg ALN-AGT01 SC once every 6 months during the 12-month DB period and DB Extension period. Patients randomized to receive placebo will be randomized to 1 of the 4 initial dose regimens of ALN-AGT01 beginning at Month 6.

### Reference Treatment, Dose, and Mode of Administration

Placebo (sodium chloride 0.9% w/v for SC administration) will be administered once every 3 months and at the same volume as the study drug. Patients receiving once every 6 months

ALN-AGT01 regimens will receive placebo SC at dosing visits at which they do not receive ALN-AGT01 to maintain the blind.

### Duration of Treatment and Study Participation

The duration of treatment with ALN-AGT01 is up to 24 months. The estimated total time on study for each patient is up to 44 months, including up to 2 months of screening, followed by up to 24 months of treatment, and up to 18 months in the Follow-up period.

### Study Rationale

Study ALN-AGT01-002 (KARDIA-1) is a randomized, double-blind, placebo-controlled, dose-ranging, multicenter Phase 2 study designed to evaluate the safety, efficacy, and pharmacodynamics (PD) of ALN-AGT01, administered SC, in patients with mild-to-moderate hypertension. Patients will be randomized to 1 of 4 ALN-AGT01 treatment regimens or placebo for the first 6 months of the 12-month Double-blind (DB) period. After the first 6 months of the DB period, patients from the placebo arm will be re-randomized to 1 of the 4 initial

ALN-AGT01 regimens for the remaining 6 months of the DB period, while patients randomized to ALN-AGT01 will remain on their originally assigned regimens. After completion of the 12-month DB period, patients may be eligible to participate in an ALN-AGT01 open-label extension (OLE) study. If an individual patient reaches Month 12 prior to availability of the OLE study, they may continue their current blinded dosing in the DB Extension period for up to 12 additional months until the OLE study is open and then transition.

The primary objective of the study is to evaluate the efficacy of ALN-AGT01 for the treatment of hypertension by evaluating the impact on systolic blood pressure (SBP) from baseline to Month 3, as assessed by ambulatory blood pressure monitoring (ABPM). Secondary and exploratory objectives of the study include evaluating the efficacy of ALN-AGT01 on other measures of blood pressure response and evaluating the PD effect of ALN-AGT01, including reduction in circulating AGT concentration.

Objectives and Endpoints

| Objectives | Endpoints |
|---|---|
| Primary | |
| To evaluate the effect of ALN-AGT01 on | Change in SBP from baseline to Month 3, |
| SBP as assessed by ABPM at Month 3 | assessed by ABPM |
| Secondary | |
| Through Month 6 | Change in SBP and DBP assessed by |
| To evaluate the effect of ALN-AGT01 on | ABPM |
| blood pressure assessed by ABPM | |
| To evaluate the effect of ALN-AGT01 on | Change in office SBP and DBP |
| office blood pressure | |
| To characterize the PD effects of | Change in serum AGT |
| ALN-AGT01 | |
| Exploratory | |
| To evaluate the effect of ALN-AGT01, | Change in SBP and DBP assessed by |
| over time, on other measures of blood | ABPM |
| pressure reduction (through Month 12) | Change in office SBP and DBP |
| | Office blood pressure and ABPM |
| | response rate (by blood pressure |
| | reduction) |
| | Office blood pressure and ABPM and |
| | response rate (by blood pressure |
| | normalization) |
| | Proportion of patients with oral |
| | antihypertensive use |
| | Change in SBP and DBP assessed by |
| | HBPM |
| | Change in daytime and nighttime blood |
| | pressure (including dipping pattern) |
| To characterize the PD effects of | Change in serum AGT |
| ALN-AGT01 (after Month 6) | |
| To characterize the plasma PK of | Plasma concentrations of ALN-AGT01 |
| ALN-AGT01 | and its metabolite AS(N-1)3′ |
| | ALN-AGT01 |
| To assess the effect of ALN-AGT01 on | Change from baseline in plasma renin |
| exploratory biomarkers of the RAAS | concentration, aldosterone, AngI, and |
| | AngII |
| To evaluate the immunogenicity of | Incidence and titers of ADA |
| ALN-AGT01 | |
| To assess the effect of ALN-AGT01 on | Change from baseline in body weight, |
| body weight, BMI, and morphometric | BMI, waist circumference, and waist-to- |
| measurements | hip ratio |
| To assess the effect of ALN-AGT01 on | Change from baseline in HbAlc, fasting |
| metabolic syndrome parameters | plasma glucose, insulin, and serum lipid |
| | profile |
| To correlate blood pressure measurements | Correlation of blood pressure values |
| obtained with a wearable, noninvasive, | obtained with a wearable device versus |
| cuffless device to those obtained by | ABPM, HBPM, and office measurements |
| standard cuff-based methods | |
| To assess the long-term treatment effect | Change from baseline in SBP and DBP |
| of ALN-AGT01 (through Month 24) | assessed by ABPM, office blood pressure, |
| | and HBPM |
| Safety | |
| To evaluate the safety of ALN-AGT01 in | Frequency of AEs |
| patients with mild to moderate | |
| hypertension | |

Abbreviations: ABPM = ambulatory blood pressure monitoring; ADA = anti-drug antibody; AE = adverse event; AGT = angiotensinogen; Ang = angiotensin; BMI = body mass index; DBP = diastolic blood pressure; ECG = electrocardiogram; HbA1c = hemoglobin A1C; HBPM = home blood pressure monitoring; PD = pharmacodynamic; PK = pharmacokinetic; RAAS = renin-angiotensin-aldosterone system; SBP = systolic blood pressure.

### Summary of Study Design

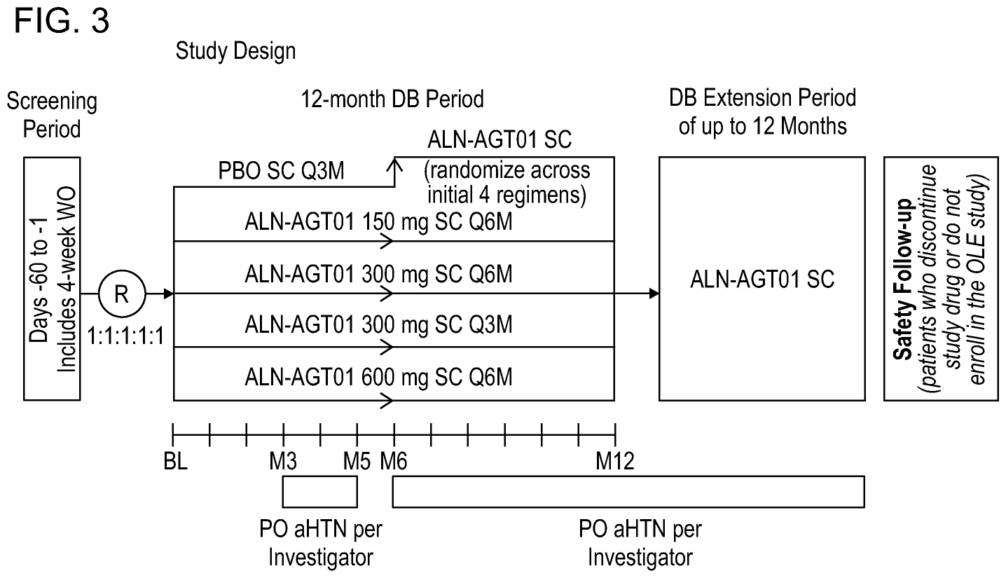

This is a Phase 2, randomized, double-blind, placebo-controlled, dose-ranging, multicenter study designed to evaluate the safety, efficacy, and PD of ALN-AGT01, administered SC, in patients with mild-to-moderate hypertension. A schematic of the study design is provided in FIG. 3.

Before randomization, patients will discontinue prior antihypertensive medications (if taking) for a Washout period of at least 4 weeks.

Patients who complete the Screening period and meet all inclusion/exclusion criteria for enrollment will be randomized 1:1:1:1:1 to receive 1 of the following regimens over a 12-month DB treatment period. Randomization will be stratified by race (black vs all other races) and by baseline blood pressure (mean 24-hour SBP< or ≥145 mmHg).
Placebo SC once every 3 months, with re-randomization (1:1:1:1) at Month 6 to 1 of the initial 4 ALN-AGT01 regimens
150 mg ALN-AGT01 SC once every 6 months
300 mg ALN-AGT01 SC once every 6 months
300 mg ALN-AGT01 SC once every 3 months
600 mg ALN-AGT01 SC once every 6 months

Patients receiving once every 6 month ALN-AGT01 regimens will receive placebo SC at dosing visits at which they do not receive ALN-AGT01 to maintain the blind.

At Month 3, conventional oral antihypertensives may be added for daytime mean SBP≥135 mmHg by ABPM. After Month 3, oral antihypertensive medications may also be added for persistent elevations in blood pressure above target (target defined as office SBP<140 mmHg, home blood pressure monitoring [HBPM] SBP

<135 mmHg, or daytime mean SBP<135 mmHg by ABPM). Oral antihypertensives (if taking) will be temporarily held from Month 5 to Month 6 in appropriate patients (Month 5 office SBP
<155 mmHg if taking 1 oral antihypertensive agent or office SBP<150 mmHg if taking 2
agents) to assess the effect of ALN-AGT01 alone (vs placebo) at Month 6 by ABPM. During this 4-week period, blood pressure will be carefully monitored by daily HBPM and oral antihypertensive medications restarted if confirmed office SBP>170 mmHg (or if confirmed office SBP>160 mmHg accompanied by symptoms).

Patients may be eligible to participate in an ALN-AGT01 OLE study upon completion of the Month 12 predose assessments. If an individual patient reaches Month 12 prior to availability of the OLE study, they may continue their current blinded dosing in the DB Extension period for up to 12 additional months until the OLE study is open and then transition. Once the OLE study is open for enrollment, eligible patients may rollover into the OLE study at Months 12, 18, or 24 (whichever visit occurs first).

In the DB Extension period, blood pressure will be closely monitored and individual modification of antihypertensive therapy will be allowed to maintain blood pressure in target range.

### Inclusion Criteria

Patients are eligible to be included in the study if all the following criteria apply:

### Age and Sex

1. Age 18 to 75 years, inclusive
2. Male or female

### Patient and Disease Characteristics

3. Has untreated hypertension (not taking antihypertensive medication) or is on stable therapy with 1 or more antihypertensive medications of the following classes: an ACE inhibitor, ARB, renin inhibitor, CCB, thiazide diuretic, and/or thiazide-like diuretic. In general, stable therapy is defined as having no change in antihypertensive medication or dose within 30 days prior to screening.
4. Daytime mean SBP≥135 mmHg and ≤160 mmHg by ABPM, without antihypertensive medication. Patients previously taking medication for hypertension must be without antihypertensives for ≥4 weeks prior to this ABPM.
5.

### Exclusion Criteria

Patients are excluded from the study if any of the following criteria apply:

### Disease-Specific Conditions

1. Secondary hypertension
2. Orthostatic hypotension (symptomatic or asymptomatic), defined as a fall of ≥20 mmHg SBP or ≥10 mmHg DBP within approximately 1 to 3 minutes of standing up from a seated position by office blood pressure.

### Laboratory Assessments

3. Has any of the following laboratory parameter assessments after at least 4 weeks of washout:
   a. ALT or aspartate aminotransferase (AST)>2× upper limit of normal (ULN)
   b. Total bilirubin >1.5×ULN. Patients with elevated total bilirubin that is secondary to documented Gilbert's syndrome are eligible if the total bilirubin is <2×ULN
   c. International normalized ratio (INR)>2.0 (patients on oral anticoagulant [e.g., warfarin] with an INR<3.5 will be allowed)
   d. Elevated potassium >5 mEq/L
e. eGFR of ≤30 m/min/1.73 m² (calculation will be based on the Modification of Diet in Renal Disease formula)

### Prior/Concomitant Therapy

4. Received an investigational agent within the last 30 days before randomization or are in follow-up of another clinical study prior to study enrollment. Any agent that has received health agency authorization (including for emergency use) by local or regional regulatory authorities is not considered investigational.
5. Currently taking, taken within 30 days prior to randomization, or anticipated to receive during the course of the study any medication or herbal supplement known to significantly affect blood pressure (with the exception of medications for the treatment of essential hypertension). Patients who require medications such as monoamine oxidase (MAO) inhibitors that are associated with hypertensive crisis should be excluded. Whelton P K, Hypertension. 2018 June;71(6):e13-e115
6. Currently taking, or taken within 30 days prior to randomization, beta blockers
7. Currently taking, taken within 30 days prior to randomization, or anticipated to receive during the course of the study sodium-glucose co-transporter 2 (SGLT2) inhibitors
8. Prescription nonsteroidal anti-inflammatory drugs (NSAIDs) are not permitted. In addition, chronic/standing use of over-the-counter NSAIDs is not permitted.

Paracetamol/acetaminophen (up to 2 g per day) for analgesia will be allowed.
9. Anticipates using organic nitrate preparations (eg, nitroglycerin, isosorbide mononitrate, isosorbide dinitrate, pentaerythritol) during the course of the study
10. Received an RNAi therapeutic (approved or investigational) within 6 months prior to randomization

### Medical Conditions

11. Current or prior history of intolerance to an ARB, ACE inhibitor (other than cough), or direct renin inhibitor
12. Medical condition, other than hypertension, that requires treatment with a RAAS inhibitor
13. History of multiple drug allergies or history of allergic reaction to an oligonucleotide or to GalNAc
14. Type 1 diabetes mellitus, poorly controlled Type 2 diabetes mellitus (hemoglobin A1c [HbA1c]>8.0%), newly diagnosed Type 2 diabetes mellitus (within 6 months prior to randomization), or laboratory evidence of diabetes during screening (fasting plasma glucose ≥126 mg/dL [7.0 mmol/L], random plasma glucose ≥200 mg/dL [11.1 mmol/L], or HbA1c≥6.5%) without known diagnosis of diabetes
15. History of severely increased albuminuria (urine albumin:creatinine ratio >300 mg/g or >300 mg/day) or laboratory results consistent with this diagnosis upon screening
16. Has known human immunodeficiency virus or evidence of current or chronic hepatitis C virus (HCV) or hepatitis B virus (HBV) infection
17. History of any cardiovascular event (eg, stroke, transient ischemic attack, myocardial infarction, unstable angina, coronary artery bypass grafting, percutaneous coronary intervention, hospitalization due to heart failure) within 6 months prior to randomization
18. Clinically significant valvular heart disease
19. New York Heart Association II to IV heart failure
20. Uncontrolled serious cardiac arrhythmia, defined as recurrent and highly symptomatic ventricular tachycardia, atrial fibrillation with rapid ventricular response, or supraventricular tachycardia in the 3 months prior to randomization
21. Has undergone liver transplantation or is anticipated to be on an active liver transplantation waiting list during the study treatment period
22. History of renal transplantation or under immunosuppressive therapy
23. Has other medical conditions or comorbidities which, in the opinion of the Investigator, would interfere with study compliance or data interpretation; or, in the opinion of the Investigator, taking part in the study would jeopardize the safety of the patient
24. Clinically significant illness, in the opinion of the Investigator, within 7 days prior to randomization
25. Known change in body weight >10% in last 6 months prior to screening
26. History of intolerance to SC injection(s) that could potentially hinder study drug administration or evaluation of local tolerability
27. Has planned major surgery or general anesthesia during the study

### Contraception, Pregnancy, and Breastfeeding

28. Is not willing to comply with the contraceptive requirements during the study period
29. Female patient is pregnant, planning a pregnancy, or breast-feeding.

### Alcohol or Nicotine Use and Substance Abuse

30. Unwilling or unable to limit alcohol consumption throughout the course of the study. Alcohol intake of >2 units/day is excluded during the study (unit: 1 glass of wine [approximately 125 mL]=1 measure of spirits [approximately 1 fluid ounce]=½ pint of beer [approximately 284 mL]).
31. History of alcohol or substance abuse (licit or illicit drugs) within the last 12 months before screening, in the opinion of the Investigator
32. Unwilling or unable to abstain from use of nicotine or tobacco-containing products (including but not limited to snuff, chewing tobacco, cigars, cigarettes, pipes, nicotine patches, or e-cigarettes) within 30 minutes prior to office blood pressure measurements

### Other Restrictions

33. Third shift or night shift workers
34. Arm circumference exceeds the maximum cuff size of any of the blood pressure instruments provided by the Sponsor
35. Unable or unwilling to perform HBPM as specified

### Efficacy Analyses

The primary endpoint is the change in SBP from baseline at Month 3 assessed by ABPM. The primary hypothesis of the dose response relationship for the primary endpoint across ALN-AGT01 doses and placebo will be tested using a multiple comparison-modeling (MCP Mod) approach.[Bretz F, Pinheiro J C,Branson M. Combining multiple comparisons and modeling techniques in dose-response studies. Biometrics. 2005 September;61(3):738-48] The presence of a dose-response trend will be initially tested against a set of prespecified dose-response models at a 2-sided significance level of 0.05, adjusted for multiplicity (the MCP step). Several candidate models will be prespecified in the SAP. Then, the dose-response curves will be further estimated (the modeling step) based on the ‘best’ fitted dose response model. Furthermore, each ALN-AGT01 dose group will be compared against placebo using Dunnett's Test.

For the secondary endpoints of change in DBP assessed by ABPM at Month 3 and change in SBP and DBP assessed by ABPM at Month 6, each ALN-AGT01 dose group will be compared with placebo. For change in office SBP and DBP, each ALN-AGT01 dose group will be compared with placebo at Month 3 and Month 6 and also using the time-adjusted average from Month 1 to Month 3 and from Month 1 to Month 6.

No multiplicity adjustment is applied across primary and secondary endpoints.

### Pharmacodynamic Analysis

Pharmacodynamic analyses will include the evaluation of changes in levels of serum AGT and other exploratory biomarkers of the RAAS pathway. Descriptive statistics for observed levels and the relative change from baseline for all measured biomarkers will be presented for each of the postdose time points.

Statistical comparison of the biomarker levels (absolute and/or change from baseline) across treatment groups may be explored. Details of the analysis will be specified in the SAP.

Population PK/PD analysis may be conducted to evaluate the dose-response relationships for PD lowering after ALN-AGT01 treatment. Additionally, the relationship between lowering of serum AGT and blood pressure may be explored within a modeling framework. If conducted, these analyses will be described in a pharmacometrics analysis plan and results will be presented in a separate report.

### Pharmacokinetic Analysis

Plasma concentrations of ALN-AGT01 and its metabolite AS(N-1)3′ ALN-AGT01 will be summarized using descriptive statistics.

Population PK analysis may be conducted on the PK data from this study. If conducted, the analysis methods will be described in a pharmacometrics analysis plan and results will be presented in a separate report.

### Safety Analyses

The primary parameter is the frequency of treatment-emergent AEs (hereafter referred to simply as AEs). Safety parameters also include vital signs, ECGs, clinical laboratory assessments and physical exams. Extent of exposure will be summarized.

Prior and concomitant medications will be coded using the World Health Organization Drug Dictionary. Results will be tabulated by Anatomical Therapeutic Chemical Classification System and Preferred Term (PT).

Adverse events will be classified according to the Medical Dictionary for Regulatory Activities (MedDRA) System Organ Class (SOC) and PT. Adverse events, SAEs, related AEs, AEs leading to discontinuation of study drug, and AEs leading to death will be summarized by SOC and PT for each treatment arm. By-patient listings will be provided for deaths, SAEs, and AEs leading to discontinuation of study drug.

Descriptive statistics will be provided for clinical laboratory parameters, ECG, and vital signs summarizing the observed values and changes from baseline over time. Laboratory shift tables from baseline grade (or category) to worst post-baseline grade (or category) will be presented for laboratory parameters that are graded or categorized. Abnormal physical exam findings will be presented in listings.

### Example 3. Durable Reductions in Circulating AGT and Blood Pressure 6 Months after Single Doses of AD-85481 (Zilebesiran) in Hypertensive Patients

A multicenter, randomized, double-blinded, placebo-controlled, single ascending fixed dose study was performed to assess the safety and efficacy of AD-85481 (Zilebesiran) at 6 months after single fixed dose administration to subjects having hypertension. (ClinicalTrials.gov Identifier: NCT03934307, Part A).

This study included adults 18 to 65 years of age with hypertension (subjects having systolic blood pressure (SBP) of >130 and ≤165 mmHg) without antihypertensive medications, with a 24 hour ambulatory blood pressure monitoring (ABPM) of ≥130 mmHg, and a BMI≥18 and ≤35 kg/m2. Patients with secondary hypertension were excluded. Patients were treatment naïve or, if on prior antihypertensive medication were washed out for ≥2 weeks before screening.

A total of 84 patients with hypertension were enrolled in this ascending single fixed dose study of zilebesiran up to 800 mg. The demographics and baseline characteristics of subjects are presented in the table below.

| | | AD-85481 Dose Cohort | | | | | | | All | |
|---|---|---|---|---|---|---|---|---|---|---|
| | | 10 | 20 | 50 | 100 | 200 | 400 | 800 | AD- | |
| | Placebo | mg | mg | mg | mg | mg | mg | mg | 85481 | |
| Characteristic | (N = 28) | (N = 8) | (N = 8) | (N = 8) | (N = 8) | (N = 8) | (N = 8) | (N = 8) | (N = 56) | |
| Age, years; median | | 52 | 53 | 56 | 41 | 56 | 56 | 58 | 61 | 55 |
| (range) | | (36, 64) | (37, | (47, | (35, | (35, | (43, | (44, | (45, | (35, |
| | | | 60) | 63) | 64) | 65) | 64) | 64) | 62) | 65) |
| Gender | Male | 16 | 7 | 2 | 7 | 3 | 5 | 7 | 4 | 35 |
| | Female | 12 | 1 | 6 | 1 | 5 | 3 | 1 | 4 | 21 |
| Race | White | 21 | 6 | 4 | 3 | 4 | 6 | 6 | 6 | 35 |
| | Black | 6 | 1 | 4 | 4 | 2 | 2 | 1 | 2 | 16 |
| | Asian | 0 | 1 | 0 | 0 | 2 | 0 | 0 | 0 | 3 |
| | Other | 1 | 0 | 0 | 1 | 0 | 0 | 1 | 0 | 2 |
| Blood | 24 h | 142 | 139 | 140 | 135 | 137 | 139 | 138 | 142 | 137 |
| Pressure | SBP | (126, | (130, | (132, | (113, | (131, | (129, | (132, | (131, | (113, |
| (by | median | 153) | 147) | 157) | 144) | 152) | 154) | 160) | 167) | 167) |
| ABPM, | (range) | | | | | | | | | |
| mmHg) | 24 h | 88 | 84 | 91 | 83 | 86 | 83 | 90 | 88 | 85 |
| | DBP | (72, | (76, | (75, | (74, | (80, | (75, | (76, | (75, | (74, |
| | median | 103) | 93) | 103) | 91) | 90) | 95) | 99) | 102) | 103) |
| | (range) | | | | | | | | | |

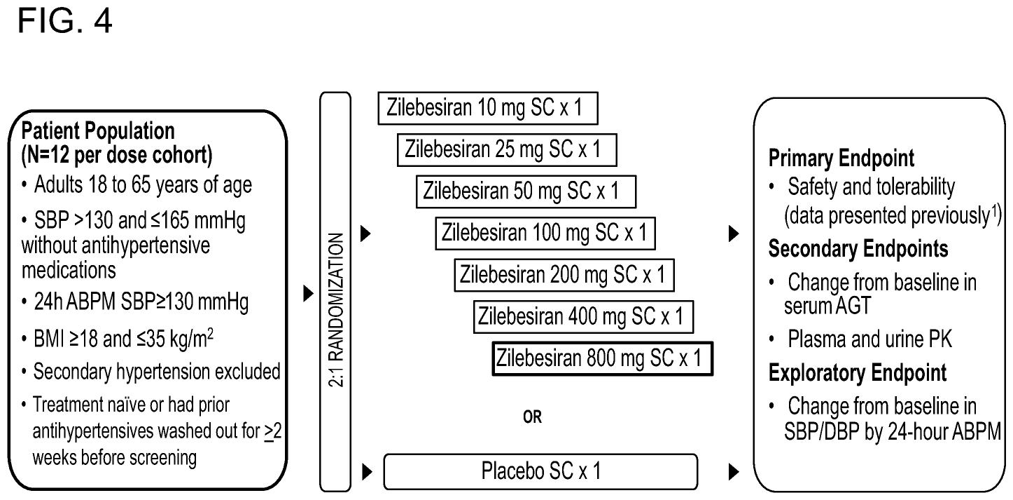

Patients were subcutaneously administered placebo or a single 10 mg, 25 mg, 50 mg, 100 mg, 200 mg, 400 mg or 800 mg fixed dose of AD-85481 (FIG. 4).

The safety and tolerability, change from baseline in serum AGT, and plasma and urine pharmacokinetic parameters were evaluated. Change from baseline in blood pressure was also assessed by 24-hour ABPM. Specifically, change from baseline blood pressure was measured by ambulatory blood pressure monitoring (ABPM) at week 8, week 12, and week 24. After week 8, if a patient developed clinically significant elevated blood pressure, add-on treatment was initiated. Patients were unblinded after Week 12. Patients treated with AD-85481 entered a follow-up period and patients treated with placebo were not required to complete a safety follow-up period.

Safety and tolerability was evaluated per protocol during the double-blinded phase (12-week timepoint). AD-85481 administration was generally well tolerated with no treatment-related serious adverse events (SAEs). Most adverse events (AEs) were mild or moderate in severity and were resolved without intervention. No death or AEs leading to study withdrawal were reported. A severe and serious AE of prostate cancer was reported in one patient who received 200 mg AD-85481, based on a biopsy that was performed during the screening period and reported as positive after dosing. A severe and serious AE of optic ischemic neuropathy was reported in one patient who received placebo. No additional SAEs were observed through Week 24. No patient required intervention for low blood pressure. No clinically significant elevations in serum ALT, serum creatinine or serum potassium were reported. Five patients treated with AD-85481 had injection site reactions, all mild and transient.

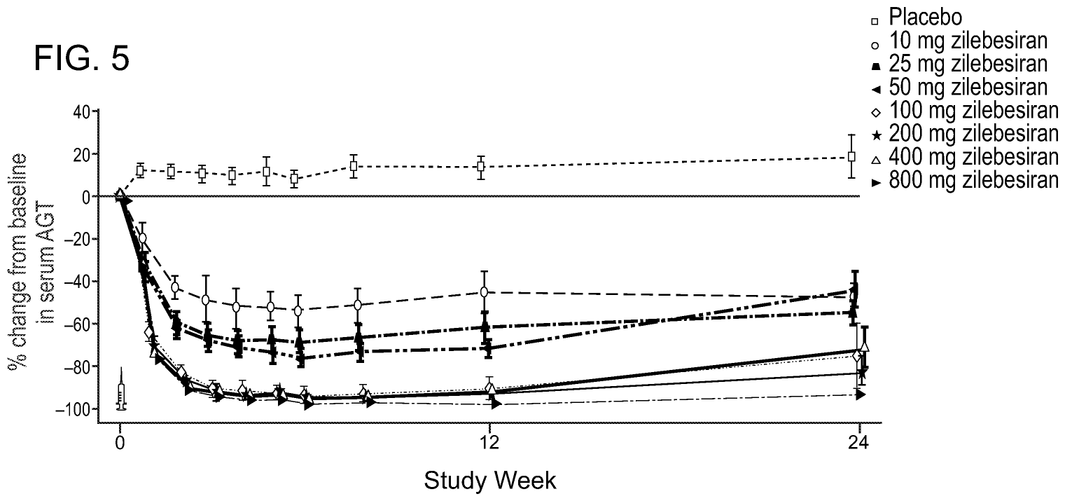

The concentration of serum AGT was measured after single doses of AD-85481. As shown in FIG. 5, a reduction of ≥90% in serum AGT from baseline was observed with single doses of AD-85481 ≥100 mg from Week 3 which were sustained to Week 12. All patients who received a single 800 dose of AD-85481 maintained reduction of ≥90% in serum AGT to Week 24.

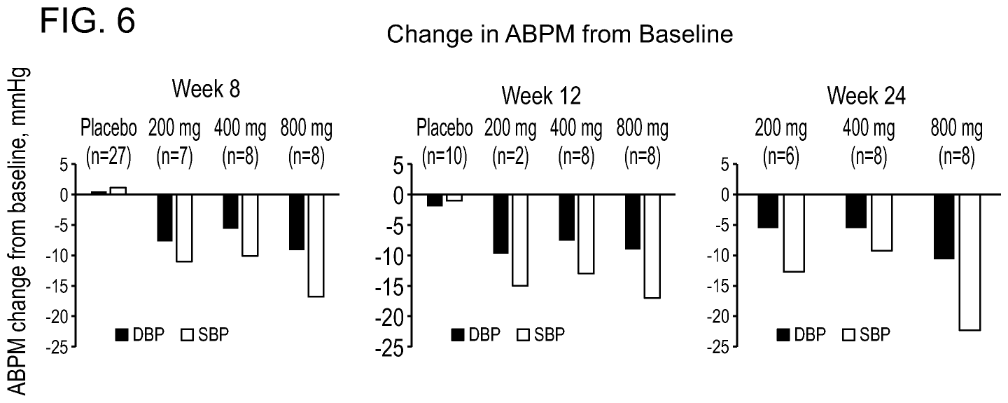

Assessments of AD-85481 also included change from baseline in SBP and DBP determined by 24 hour ambulatory blood pressure monitoring (ABPM). A durable antihypertensive effect of single dose administration of AD-85481 was demonstrated. As shown in FIG. 6, a mean 24-hour SBP reduction of >10 mmHg was achieved at Week 8 across dose groups receiving a fixed dose of ≥200 mg and clinically meaningful reductions in BP were maintained through Week 24. After a single 800 mg dose of AD-85481, a mean 24-h SBP reduction of >20 mmHg was observed at Week 24. Of the 8 patients in this group (800 mg), 6 achieved a mean 24-h SBP reduction of >20 mmHg at Week 24 without add-on antihypertensives. In addition, serum AGT reduction correlated with 24-hour SBP reduction across multiple doses.

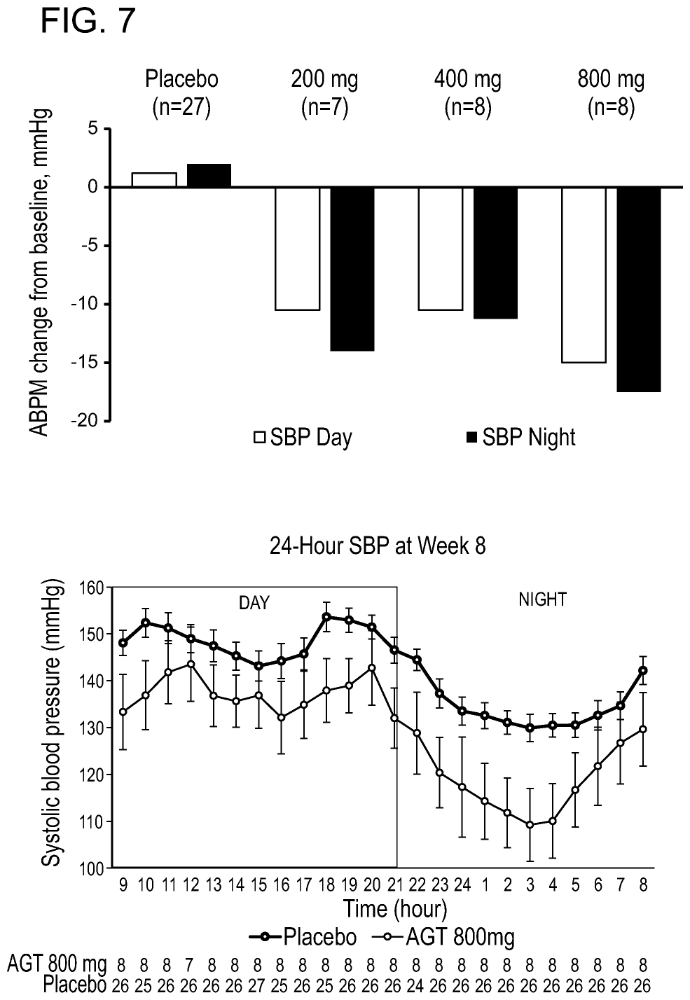

Consistent blood pressure reductions over a 24 hour period were also observed. Marked reductions in both daytime and nighttime SBP were demonstrated with AD-85481 doses ≥200 mg at Week 8 (FIG. 7). These reductions in daytime and nighttime SBP were sustained to later timepoints (Weeks 12 and 24; data not shown). Similar improvements during daytime and nighttime were also seen for DBP (data not shown)

In conclusion, a single subcutaneously administered fixed dose of AD-85481 was well tolerated in patients with mild-to-moderate hypertension, supporting its continued development in patients with hypertension. Sustained reduction in serum AGT>90% and mean reduction in 24-hour SBP>20 mmHg were observed after a single 800 mg fixed dose of AD-85481. AD-85481 led to >10 mmHg reduction in 24-hour SBP at Week 8 across dose groups ≥200 mg, with the clinically meaningful BP reduction maintained through Week 24. AD-85481 produced consistent and sustained reduction in BP during both daytime and nighttime. These data support further evaluation of both quarterly and biannual dose administration of AD-85481 in hypertension.

### Example 4. Safety and Tolerability of AD-85481 (Zilebesiran) in Hypertensive Patients During Sodium Depletion

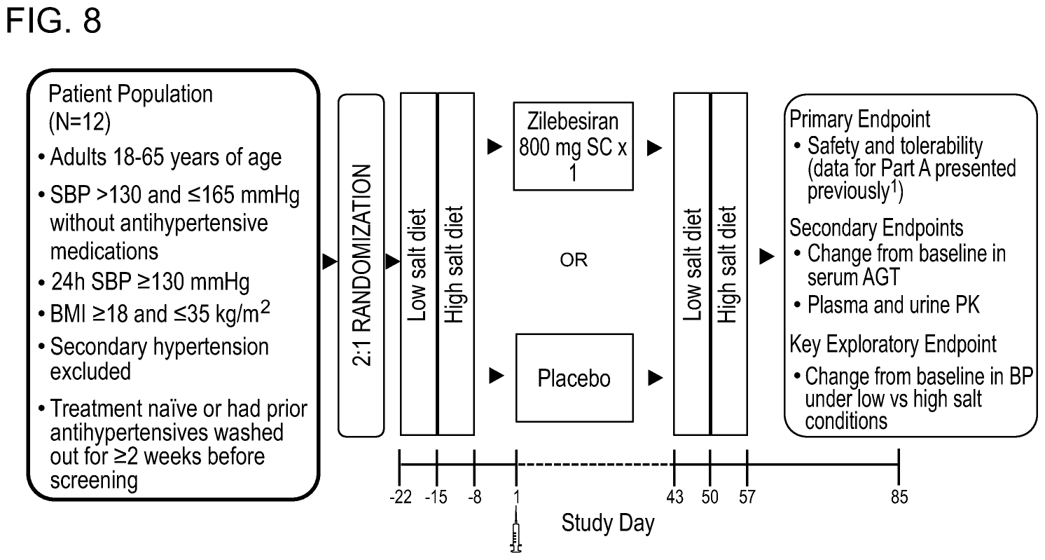

In a multicenter, randomized, double-blinded, placebo-controlled, single ascending fixed dose study to assess the safety and efficacy of AD-85481 (Zilebesiran) after single fixed dose administration to subjects having hypertension (ClinicalTrials.gov Identifier: NCT03934307, Part A), a two-week dietary sub-study varying sodium consumption was performed (NCT03934307, Part B). The purpose of this study was to identify any potential salt-sensitive blood pressure responses to AD-85481 treatment (FIG. 8). Briefly, a low salt diet (0.23 g sodium per day) was given to subjects pre-dose at Day −22 to Day −15, and post-dose at Day 43 to Day 50. A high salt diet (5.75 g sodium per day) was given to the subjects pre-dose at Day −15 to Day −8, and post-dose at Day 50 to Day 57.

Patients received either placebo (n=4 per cohort) or a single subcutaneously administered 800 mg fixed dose of AD-85481 (n=8 per cohort). The demographics and baseline characteristics of the subjects are presented in the table below.

| | | Placebo | Zilebesiran |
|---|---|---|---|
| Characteristic | | (n = 4) | (n = 8) |
| Age, years; | | 52 (42, | 60 (58, |
| median (range) | | 58) | 62) |
| Gender | Male (%) | 3 (75) | 6 (75) |
| Race | White | 3 | 5 |
| | Black | 1 | 2 |
| | Asian | 0 | 1 |
| Blood Pressure | 24 h SBP | 147 (133, | 138 (129, |
| (ABPM, mmHg) | median (range) | 151) | 150) |
| | 24 h DBP | 98 (87, | 88 (77, |
| | median (range) | 103) | 93) |

The safety and tolerability, change from baseline in serum AGT, and plasma and urine pharmacokinetic parameters were evaluated. Change from baseline in blood pressure was also evaluated under low and high salt conditions.

Safety was reported from the start of the study to Day 85. The safety profile of AD-85481 was acceptable with no safety concerns. All adverse events were mild in severity and were resolved without intervention. There was neither death nor serious adverse events (SAEs) reported, and no adverse events (AEs) leading to study withdrawal. There was no AEs of injection site reaction nor hypotension, and no patients required intervention for low blood pressure, including during the sodium deprivation period. No clinically significant elevations in serum alanine aminotransferase (ALT), serum creatinine, or serum potassium in AD-85481 treated group were reported. One placebo-treated patient had transient ALT elevation >3× upper limit of normal (ULN) attributed to alcohol consumption

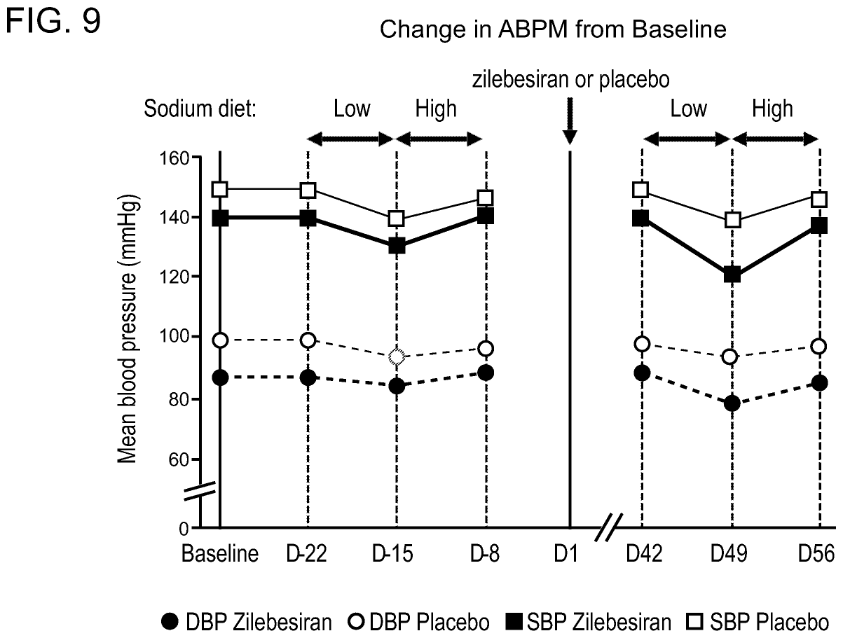

Assessments of AD-85481 also included change from baseline in SBP and DBP determined by 24 hour ambulatory blood pressure monitoring (ABPM). Reduction in serum AGT levels of >90% was achieved and sustained between Week 2 and Week 12 following a single dose of 800 mg AD-85481 alone (data now shown). As depicted in FIG. 9, a reduction in 24-hour SBP and DBP was observed pre-dose for all patients following a low-salt diet. Blood pressure increased upon switching to a high-salt diet. After administering AD-85481 or placebo, blood pressure changes were more profound following a low-salt diet for AD-85481 versus placebo-treated patients, whereas a high-salt diet modulated the blood pressure lowering effect of AD-85481.

### Example 5. A Safety and Tolerability of AD-85481 (Zilebesiran) in Hypertensive Patients During Irbesartan Co-Administration

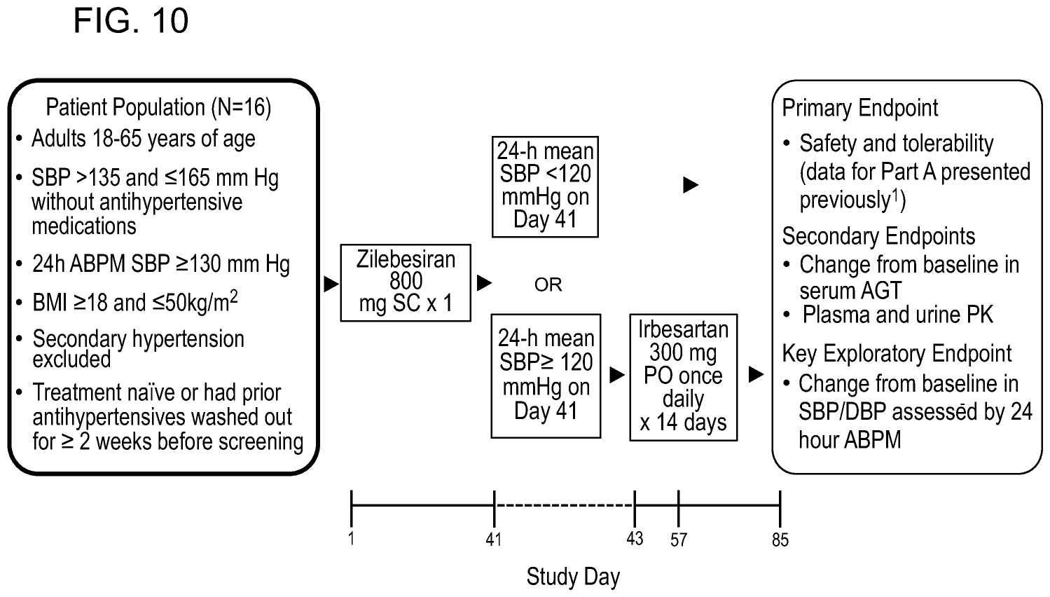

In a multicenter, randomized, double-blinded, placebo-controlled, single ascending fixed dose study to assess the safety and efficacy of AD-85481 (Zilebesiran) after a single fixed dose administration to subjects having hypertension (ClinicalTrials.gov Identifier: NCT03934307, Part A), a sub-study was performed to assess the safety and tolerability of AD-85481 (Zilebesiran) in hypertensive patients during irbesartan co-administration (NCT03934307, Part E). Briefly, all patients received an open-label single 800 mg fixed dose of AD-85481 by subcutaneous administration on Day 1. On Day 41, patients with a 24-hr mean systolic blood pressure (SBP)≥120 mmHg (N=10) proceeded to receive irbesartan (300 mg oral) once daily from Day 43 to Day 57 for 14 days (FIG. 10).

The demographics and baseline characteristics of subjects are presented in the table below.

| | | Zilebesiran | Irbesartan |
|---|---|---|---|
| Characteristic | | Only (N = 6) | Add-On (N = 10) |
| Age, years; | | 56 (44- | 56 (42- |
| median (range) | | 58) | 64) |
| Gender | Male (%) | 5 (83) | 3 (30) |
| Race | White | 6 | 4 |
| | Black | 0 | 3 |
| | Asian | 0 | 1 |
| | Other | 0 | 2 |
| Blood Pressure | 24 h SBP | 135 (124- | 146 (135- |
| (ABPM, mmHg) | median (range) | 141) | 158) |
| | 24 h DBP | 83 (78- | 89 (76- |
| | median (range) | 98) | 99) |

The safety and tolerability, change from baseline in serum AGT, and plasma and urine pharmacokinetic parameters were evaluated. Change from baseline in blood pressure was also assessed by 24-hour ABPM.

Safety was reported from the start of the study to Day 85. The safety profile of AD-85481 was generally well tolerated with no drug-related serious adverse events (SAEs). All adverse events were mild in severity. There was one mild SAE of acute anemia in the irbesartan add-on group, but was considered not related to study drug, which was a complication of esophagogastroduodenoscopy with biopsy performed during screening prior to dose of zilebesiran. There were no AEs of concern for hypotensive events during irbesartan administration. There were also no patient required intervention for low blood pressure. No clinically significant elevations in serum ALT, serum creatinine or serum potassium were reported

Assessments of AD-85481 also included change from baseline in SBP and DBP determined by 24 hour ambulatory blood pressure monitoring (ABPM). Reduction in serum AGT levels of >90% were achieved and sustained between Week 2 and Week 12 following a single dose of 800 mg AD-85481 alone (data now shown). Add-on irbesartan 300 mg orally daily had no additional effect on serum AGT levels. As shown in the Table below, a single subcutaneously administered 800 mg fixed dose of zilebesiran reduced both systolic and diastolic BP (Day 1-41), and daily add-on irbesartan for 2 weeks further reduced systolic and diastolic BP (Day 43-57).

| | Δ24 h SBP | | | Δ24 h DBP | | |
|---|---|---|---|---|---|---|
| | mmHg; mean (SEM) | | | mmHg; mean (SEM) | | |
| | | | Zilebesiran + | | | Zilebesiran + |
| | Zilebesiran | | Irbesartan | Zilebesiran | | Irbesartan |
| Time Period | (N = 6) | | (N = 10) | (N = 6) | | (N = 10) |
| Day 1 | −22.0 | (2.9) | −7.7 (2.5) | −14.3 | (2.3) | −3.3 (1.0) |
| (Baseline) | | | | | | |
| to Day 41 | | | | | | |
| Day 43 | 0.4 | (2.9) | −6.4 (3.1) | −0.5 | (1.6) | −3.2 (1.9) |
| to Day 57 | | | | | | |

### Summary of Examples 4 and 5

Single subcutaneously administered 800 mg fixed doses of zilebesiran were generally well-tolerated in patients with mild to moderate hypertension, with no AE of hypotension reported during the low-salt diet or co-administration with irbesartan. In addition, a high-salt diet was shown to modulate the BP-lowering effect of zilebesiran, providing evidence that the standard intervention could be effective to treat potential hypotensive adverse events. Add-on irbesartan treatment with zilebesiran further reduced BP without clinically significant changes in creatinine or potassium.

### Equivalents

Those skilled in the art will recognize or be able to ascertain using no more than routine experimentation, many equivalents to the specific embodiments and methods described herein. Such equivalents are intended to be encompassed by the scope of the following claims.

1. A method selected from the group consisting of:
(i) a method for inhibiting the expression of an angiotensinogen (AGT) gene in a subject, the method comprising administering to the subject a fixed dose of about 150 mg every six months, about 300 mg every six months, about 300 mg every three months, about 600 mg every six months, about 800 mg every three months, or about 800 mg every six months of a double-stranded ribonucleic acid (RNAi) agent, or salt thereof,
wherein the double-stranded RNAi agent or salt thereof, comprises a sense strand and an antisense strand forming a double stranded region,
wherein the antisense strand comprises a nucleotide sequence comprising at least 19 contiguous nucleotides of the nucleotide sequence UGUACUCUCAUUGUGGAUGACGA of SEQ ID NO: 9, and the sense strand comprises a nucleotide sequence comprising at least 19 contiguous nucleotides of the nucleotide sequence GUCAUCCACAAUGAGAGUACA of SEQ ID NO: 10;
wherein the double-stranded RNAi agent, or salt thereof, comprises at least one modified nucleotide; and
wherein at least one of the modifications on the nucleotides is a thermally destabilizing nucleotide modification, thereby inhibiting the expression of the AGT gene in the subject;
(ii) a method for treating a subject that would benefit from reduction in angiotensinogen (AGT) expression, the method comprising administering to the subject a fixed dose of about 150 mg every six months, about 300 mg every six months, about 300 mg every 3 months, about 600 mg every six months, about 800 mg every 3 months, or about 800 mg every six months of a double-stranded ribonucleic acid (RNAi) agent, or salt thereof,
wherein the double-stranded RNAi agent, or salt thereof, comprises a sense strand and an antisense strand forming a double stranded region,
wherein the antisense strand comprises a nucleotide sequence comprising at least 19 contiguous nucleotides of the nucleotide sequence UGUACUCUCAUUGUGGAUGACGA of SEQ ID NO: 9, and the sense strand comprises a nucleotide sequence comprising at least 19 contiguous nucleotides of the nucleotide sequence GUCAUCCACAAUGAGAGUACA of SEQ ID NO: 10;
wherein the double-stranded RNAi agent, or salt thereof, comprises at least one modified nucleotide; and
wherein at least one of the modifications on the nucleotides is a thermally destabilizing nucleotide modification, thereby treating the subject that would benefit from reduction in AGT expression;
(iii) a method for treating a subject having an angiotensinogen (AGT) associated disorder, the method comprising administering to the subject a fixed dose of about 150 mg every six months, about 300 mg every six months, about 300 mg every 3 months, about 600 mg every six months, about 800 mg every 3 months, or about 800 mg every six months of a double-stranded ribonucleic acid (RNAi) agent, or salt thereof,
wherein the double-stranded RNAi agent, or salt thereof, comprises a sense strand and an antisense strand forming a double stranded region,
wherein the antisense strand comprises a nucleotide sequence comprising at least 19 contiguous nucleotides of the nucleotide sequence UGUACUCUCAUUGUGGAUGACGA of SEQ ID NO: 9, and the sense strand comprises a nucleotide sequence comprising at least 19 contiguous nucleotides of the nucleotide sequence GUCAUCCACAAUGAGAGUACA of SEQ ID NO: 10;
wherein the double-stranded RNAi agent, or salt thereof, comprises at least one modified nucleotide;
wherein at least one of the modifications on the nucleotides is a thermally destabilizing nucleotide modification, thereby treating the subject having the AGT associated disorder;
and
(iv) a method for decreasing blood pressure level in a subject, the method comprising administering to the subject a fixed dose of about 150 mg every six months, about 300 mg every six months, about 300 mg every 3 months, about 600 mg every six months, about 800 mg every 3 months, or about 800 mg every six months of a double-stranded ribonucleic acid (RNAi) agent, or salt thereof,
wherein the double-stranded RNAi agent, or salt thereof, comprises a sense strand and an antisense strand forming a double stranded region,
wherein the antisense strand comprises a nucleotide sequence comprising at least 19 contiguous nucleotides of the nucleotide sequence UGUACUCUCAUUGUGGAUGACGA of SEQ ID NO: 9, and the sense strand comprises a nucleotide sequence comprising at least 19 contiguous nucleotides of the nucleotide sequence GUCAUCCACAAUGAGAGUACA of SEQ ID NO: 10;
wherein the double-stranded RNAi agent, or salt thereof, comprises at least one modified nucleotide;
wherein at least one of the modifications on the nucleotides is a thermally destabilizing nucleotide modification, thereby decreasing the blood pressure level in the subject.

2.-24. (canceled)

25. The method of claim 1, wherein the double stranded RNAi agent, or salt thereof, is administered to the subject subcutaneously or intravenously.

26.-27. (canceled)

28. The method of claim 1, wherein:
(a) the antisense strand comprises a nucleotide sequence comprising at least 20 contiguous nucleotides of the nucleotide sequence UGUACUCUCAUUGUGGAUGACGA of SEQ ID NO: 9, and the sense strand comprises a nucleotide sequence comprising at least 20 contiguous nucleotides of the nucleotide sequence GUCAUCCACAAUGAGAGUACA of SEQ ID NO: 10;
(b) the antisense strand comprises a nucleotide sequence comprising at least 21 contiguous nucleotides of the nucleotide sequence UGUACUCUCAUUGUGGAUGACGA of SEQ ID NO: 9, and the sense strand comprises a nucleotide sequence comprising at least 20 contiguous nucleotides of the nucleotide sequence GUCAUCCACAAUGAGAGUACA of SEQ ID NO: 10;
(c) the antisense strand comprises a nucleotide sequence comprising at least 22 contiguous nucleotides of the nucleotide sequence UGUACUCUCAUUGUGGAUGACGA of SEQ ID NO: 9, and the sense strand comprises a nucleotide sequence comprising at least 20 contiguous nucleotides of the nucleotide sequence GUCAUCCACAAUGAGAGUACA of SEQ ID NO: 10;
(d) the antisense strand comprises the nucleotide sequence UGUACUCUCAUUGUGGAUGACGA of SEQ ID NO: 9, and the sense strand comprises the nucleotide sequence GUCAUCCACAAUGAGAGUACA of SEQ ID NO: 10; or
(e) the antisense strand consists of the nucleotide sequence UGUACUCUCAUUGUGGAUGACGA of SEQ ID NO: 9, and the sense strand consists of the nucleotide sequence GUCAUCCACAAUGAGAGUACA of SEQ ID NO: 10.

29.-32. (canceled)

33. The method of claim 1, wherein:
(a) substantially all of the nucleotides of the sense strand are modified nucleotides;
(b) substantially all of the nucleotides of the antisense strand are modified nucleotides;
(c) all of the nucleotides of the sense strand are modified nucleotides; or
(d) all of the nucleotides of the antisense strand are modified nucleotides.

34.-36. (canceled)

37. The method of claim 1, wherein at least one of the nucleotide modifications is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), and a 2-O—(N-methylacetamide) modified nucleotide; and combinations thereof.

38. (canceled)

39. The method of claim 1, wherein:
(a) the double stranded region is 19-23 nucleotide pairs in length, 19-21 nucleotide pairs in length, 21-23 nucleotide pairs in length, or 21 nucleotide pairs in length;
(b) each strand is independently 19-23 nucleotides in length, 19-25 nucleotides in length, or 21-23 nucleotides in length;
(c) the sense strand is 21 nucleotides in length, and the antisense strand is 23 nucleotides in length; or
(d) at least one strand comprises a 3′ overhang of at least 1 nucleotide or a 3′ overhang of at least 2 nucleotides.

40.-42. (canceled)

43. The method of claim 1, wherein the double-stranded RNAi agent, or salt thereof, further comprises at least one phosphorothioate or methylphosphonate internucleotide linkage.

44. The method of claim 43, where the phosphorothioate or methylphosphonate internucleotide linkage is at the 3′-terminus of one strand; at the 5′-terminus of one strand; or at both the 5′- and 3′-terminus of one strand.

45. The method of claim 44, wherein the one strand is the antisense strand or the sense strand.

46.-51. (canceled)

52. The method of claim 1,
wherein the antisense strand comprises a modified nucleotide sequence comprising at least 19 contiguous nucleotides of the modified nucleotide sequence usGfsuac(Tgn)cucauugUfgGfaugacsgsa of SEQ ID NO: 11, and the sense strand comprises a modified nucleotide sequence comprising at least 19 contiguous nucleotides of the modified nucleotide sequence gsuscaucCfaCfAfAfugagaguaca of SEQ ID NO: 12;
wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is a phosphorothioate linkage, thereby inhibiting the expression of the AGT gene in the subject.

53.-78. (canceled)

79. The method of claim 52, wherein;
(a) the antisense strand comprises a modified nucleotide sequence comprising at least 20 contiguous nucleotides of the modified nucleotide sequence usGfsuac(Tgn)cucauugUfgGfaugacsgsa of SEQ ID NO: 11, and the sense strand comprises a modified nucleotide sequence comprising at least 20 contiguous nucleotides of the modified nucleotide sequence gsuscaucCfaCfAfAfugagaguaca of SEQ ID NO: 12;
(b) the antisense strand comprises a modified nucleotide sequence comprising at least 21 contiguous nucleotides of the modified nucleotide sequence usGfsuac(Tgn)cucauugUfgGfaugacsgsa of SEQ ID NO: 11, and the sense strand comprises a modified nucleotide sequence comprising at least 20 contiguous nucleotides of the modified nucleotide sequence gsuscaucCfaCfAfAfugagaguaca of SEQ ID NO: 12;
(c) the antisense strand comprises a modified nucleotide sequence comprising at least 22 contiguous nucleotides of the modified nucleotide sequence usGfsuac(Tgn)cucauugUfgGfaugacsgsa of SEQ ID NO: 11, and the sense strand comprises a modified nucleotide sequence comprising at least 20 contiguous nucleotides of the modified nucleotide sequence gsuscaucCfaCfAfAfugagaguaca of SEQ ID NO: 12;
(d) the antisense strand comprises the modified nucleotide sequence usGfsuac(Tgn)cucauugUfgGfaugacsgsa of SEQ ID NO: 11, and the sense strand comprises the modified nucleotide sequence gsuscaucCfaCfAfAfugagaguaca of SEQ ID NO: 12; or
(e) the antisense strand consists of the modified nucleotide sequence usGfsuac(Tgn)cucauugUfgGfaugacsgsa of SEQ ID NO: 11, and the sense strand consists of the modified nucleotide sequence gsuscaucCfaCfAfAfugagaguaca of SEQ ID NO: 12.

80.-83. (canceled)

84. The method of claim 1, wherein the double stranded RNAi agent, or salt thereof, further comprises a ligand.

85. The method of claim 84, wherein the ligand is conjugated to the 3′ end of the sense strand.

86. The method of claim 84, wherein the ligand is an N-acetylgalactosamine (GalNAc) derivative.

87. The method of claim 86, wherein the GalNAc derivative comprises one or more GalNAc derivatives attached through a monovalent, bivalent, or trivalent branched linker.

88. The method of claim 86, wherein the ligand is

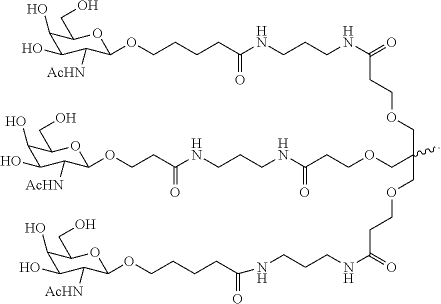 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCOC2OC(CO)C(O)C(O)C2NC(C)=O)OC(CO)C(O)C1O]

89. The method of claim 88, wherein the 3′ end of the sense strand is conjugated to the ligand as shown in the following schematic 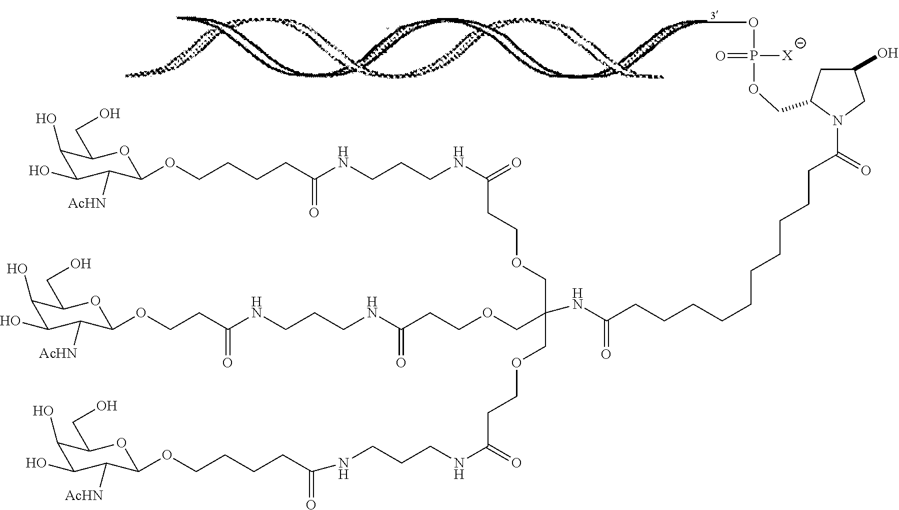
and, wherein X is 0 or S or wherein X is O.

90. The method of claim 1, wherein the subject is a human; and/or the subject is part of a group susceptible to salt sensitivity, is overweight, is obese, is pregnant, is planning to become pregnant, has type 2 diabetes, has type 1 diabetes, or has reduced kidney function.

91. The method of claim 90, wherein the subject has a systolic blood pressure of at least 130 mm Hg and a diastolic blood pressure of at least 80 mm Hg; or a systolic blood pressure of at least 140 mm Hg and a diastolic blood pressure of at least 80 mm Hg.

92.-93. (canceled)

94. The method of claim 1, wherein the subject that would benefit from reduction in AGT expression has an AGT-associated disorder.

95. The method of claim 1, wherein the AGT associated disorder is selected from the group consisting of high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; mild to moderate hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, non-alcoholic Steatohepatitis (NASH), non-alcoholic fatty liver disease (NAFLD); glucose intolerance, type 2 diabetes, and metabolic syndrome.

96. (canceled)

97. The method of claim 1, wherein the method results in:
(a) a decrease in AGT expression by at least 30%, 40% 50%, 60%, 70%, 80%, 90%, or 95%, or
(b) a decrease in systolic blood pressure and/or diastolic blood pressure of the subject.

98.-100. (canceled)

101. The method of claim 1, further comprising administering to the subject an additional therapeutic agent for treatment of hypertension.

102. The method of claim 101, wherein the additional therapeutic agent is selected from the group consisting of a diuretic, an angiotensin converting enzyme (ACE) inhibitor, an angiotensin II receptor antagonist, a beta-blocker, a vasodialator, a calcium channel blocker, an aldosterone antagonist, an alpha2-agonist, a renin inhibitor, an alpha-blocker, a peripheral acting adrenergic agent, a selective D1 receptor partial agonist, a nonselective alpha-adrenergic antagonist, a synthetic, a steroidal antimineralocorticoid agent, CCB, a thiazide diuretic, and/or a thiazide-like diuretic; a combination of any of the foregoing; and a hypertension therapeutic agent formulated as a combination of agents.

103.-106. (canceled)

107. The method of claim 1, wherein the RNAi agent, or salt thereof, is present in a pharmaceutical composition.

108.-112. (canceled)

113. A method for treating a subject having mild-to-moderate hypertension, the method comprising administering to the subject a fixed dose of about 150 mg every six months, about 300 mg every six months, about 300 mg every 3 months, about 600 mg every six months, about 800 mg every 3 months, or about 800 mg every six months of a double-stranded ribonucleic acid (RNAi) agent, or salt thereof,
wherein the double-stranded RNAi agent, or salt thereof, comprises a sense strand and an antisense strand forming a double stranded region,
wherein the antisense strand comprises a modified nucleotide sequence comprising at least 19 contiguous nucleotides of the modified nucleotide sequence usGfsuac(Tgn)cucauugUfgGfaugacsgsa of SEQ ID NO: 11, and the sense strand comprises a modified nucleotide sequence comprising at least 19 contiguous nucleotides of the modified nucleotide sequence gsuscaucCfaCfAfAfugagaguaca of SEQ ID NO: 12;
wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Tgn) is thymidine-glycol nucleic acid (GNA) S-Isomer, and s is a phosphorothioate linkage, thereby treating the subject having mild-to-moderate hypertension.

114. The method of claim 113, further comprising:
(a) measuring the levels of plasma renin, aldosterone, AngI, and/or AngII in the subject;
(b) determining blood pressure of the subject; or
(c) selecting a subject whose blood pressure is not adequately controlled by standard of care antihypertensive medications.

115. The method of claim 113, wherein:
(a) prior to administration of the double-stranded RNAi agent, or salt thereof, the subject has discontinued the administration of antihypertensive medications;
(b) the subject has been previously treated with an antihypertensive medication selected from the group consisting of an angiotensin converting enzyme inhibitor, an angiotensin II-receptor blocker, a renin inhibitor, a calcium channel blocker, a thiazide diuretic, and/or a thiazide-like diuretic;
(c) the subject has a daytime mean systolic blood pressure (SBP)≥135 mmHg and ≤160 mmHg by ABPM at least 4 weeks prior to administration of the double-stranded RNAi agent, or salt thereof; or
(d) the subject does not have secondary hypertension or orthostatic hypotension.

116. The method of claim 115, wherein the subject has discontinued the administration of antihypertensive medications for at least 2 or 4 weeks prior to administration of the double-stranded RNAi agent, or salt thereof.

117.-122. (canceled)

123. A kit for performing the method of claim 113, comprising
a) the RNAi agent, or salt thereof, and
b) instructions for use, and
c) optionally, means for administering the RNAi agent, or salt thereof, to the subject.


SEQUENCE LISTING (12 sequences)

SEQ ID NO: 1 - DNA, 2587 bp, Homo sapiens
  source 1..2587: mol_type = genomic DNA
atcccatgagcgggcagcagggtcagaagtggcccccgtgttgcctaagcaagactctcccctgccctctgccctctgcacctccggcctgcatgtccctgtggcctcttgggggtacatctcccggggctgggtcagaaggcctgggtggttggcctcaggctgtcacacacctagggagatgctcccgtttctgggaaccttggccccgactcctgcaaacttcggtaaatgtgtaactcgaccctgcaccggctcactctgttcagcagtgaaactctgcatcgatcactaagacttcctggaagaggtcccagcgtgagtgtcgcttctggcatctgtccttctggccagcctgtggtctggccaagtgatgtaaccctcctctccagcctgtgcacaggcagcctgggaacagctccatccccacccctcagctataaatagggcatcgtgacccggccgggggaagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactatctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaaccatagctggttatttctcccttgtgttagtaataaacgtcttgccacaataagcctccaaaaaaaa

SEQ ID NO: 2 - DNA, 2587 bp, Homo sapiens
  source 1..2587: mol_type = genomic DNA
ttttttttggaggcttattgtggcaagacgtttattactaacacaagggagaaataaccagctatggttccgcattcaaacagaaattcaggtgcttgcatctttcacgtattgttcaaaaatcacaagcatctgtggaaaaaactaaggtattacagacactacacggaggtcatgttcttacattcaagacactaaatacaaaccgaaggcaatgcaaaaatgtatactttaattttaaaacccaatttttgttctcaacttgaaaagggaacacttttttgtttcacaaacaagctggtcggttggaattctttttggaacagtagtcccgcgctaaacactggttcttgcctccccacccccattctctaaaataaacccagcaaactgggaggtgcatttgtgccgctgcaggcttctactgctcactccatgcagcacacttagaccaaggagaaacggctgctttccagctcaaagtcgactcattagaagaaaaggtgggagactgggggtgacacatcgctgatttgtccggggttgttatctgctgctggcctttgcctcaaaggccaggggcagaggccttgccaggcactgtgttctggggccctggcctcatgctgtgctcagcgggttggccacgcggcccaggaagtgcagggcagtggcgctttgatcatacacagcaaacaggaatgggcggttcagggtcacctccaagacctcaggcttgttaagctgttgggtagactctgtgggctctctctcatccgcttcaagctcaaaaaaaatgctgttcagcacctcccccaccctgatgcggtcattgctcaatttttgcaggttcagctcggtgtgcagaatggcgggcagctcagcctgggcgagcaggtcctgcaggtcataagatccttgcagcaccagttggggcatggtcaggtggatggtccggggagatagtttcttcatccagttgagggagttttgctggaaagtgagaccctccaccttgtccaggtcagaggcatagtgaggctggatcagcagcaggcaggcgctctcagtgaagggcacttgagtcaccgagaagttgtcctggatgtcactccagtgctggaaggtgcccatgccagagagcatgggaacagacactgaggtgctgttgtccacccagaactcctggggctcggccagcagggagaagcccttcatcttcccttggaagtggacgtaggtgttgaaagccagggtgctgtccacactggctcccatcagggagcagccagtcttccatcctgtcacagcctgcatgaacctgtcaatcttctcagcagcaacatccagttctgtgaagtccagagagcgtgggaggaccacaggggtatagagagccaggccctgcacaaacggctgcttcaggtgcaggcctggggctgtgaacacgcccaccaccgtggacagcagcagctgggcctggctatcagccctgccctgggccactagcaggccctgtacagcctgcagggcagacaggaccttgtgcgcatccagccgggaggtgcagttcttgtccttccaaggaacacccaggattgcctgtagcctgtcagctgtgtggtccaaggctcccagatagagagaggccagggtgccaaagacagccgttggggagaggacggtggccccatggaccacgccccatagctcactgtgcatgccatatatacggaagcccaagaagttggccagcatcccgaccattgcggccctcaacttgtcttcggtgtcaagttttgcagcgactagcaccagctggtcctgtagggccttttcatccacaggggatgtcttggcctgaattggagcaggtatgaaggtggggtctttgggcttcccggcattggcctttgccagctgctcacaggtactctcattgtggatgacgaggtggaaggggtgtatgtacacccggtcacctgcagccaggccagcccaggccaggaggcagaggatggtggccctcaggctcacaccggcaggagccatctcagactggggtgctcgcttccgcatacccttctgctgtagtacccagaacaacggcagcttcttcccccggccgggtcacgatgccctatttatagctgaggggtggggatggagctgttcccaggctgcctgtgcacaggctggagaggagggttacatcacttggccagaccacaggctggccagaaggacagatgccagaagcgacactcacgctgggacctcttccaggaagtcttagtgatcgatgcagagtttcactgctgaacagagtgagccggtgcagggtcgagttacacatttaccgaagtttgcaggagtcggggccaaggttcccagaaacgggagcatctccctaggtgtgtgacagcctgaggccaaccacccaggccttctgacccagccccgggagatgtacccccaagaggccacagggacatgcaggccggaggtgcagagggcagagggcaggggagagtcttgcttaggcaacacgggggccacttctgaccctgctgcccgctcatgggat

SEQ ID NO: 3 - DNA, 1834 bp, Macaca fascicularis
  source 1..1834: mol_type = genomic DNA
aagaagctgccattgttctgggtactacagcagaagggtatgcagaagcgagcaccccagtccgagatggctcctgccagcgtgagcctgagggccaccatcctctgcctcctggcctgggctggcctggccacaggtgaccgggtgtacatacaccccttccacctcgtcatccacaatgagagtacctgtgagcagctggcaaaggccgatgctgggaagcccaaagatcccaccttcacacctgttccgatacaggccaagacgtctcctgtggatgaaaaggccctgcaggaccagctagtgctggttgccgcaaaactcgacaccgaggacaagttgagagccgcgatggtcgggatgctggccaacttcttgggcttccgtatatatggcatgcacagtgagctatggggcgtggtccatggggccaccatcctctccccaacggctgtctttggcaccctggcctctctctacctgggagcgttggaccacacagccgacaggctacaggcaatcctgggcgtcccttggaaggacaagaactgcacctcccggctggatgcgcacaaggtcctctctgccctgcaggctgtacagggcctgctggtggcccagggcagggctgacggccagtcccagctgctgttgtccacagtggtgggtctcttcacagccccagatctgcacctgaagcagccgtttgtgcagggcctggctctctatgcccctgtggtcctcccacgctctctggacttcacagacctggaagtcgctgctgagaagattgacaggttcatgcaggctgtgacaggatggaagattagcagccccctgacgggagccagtgcggacagcaccctggttttcaacacctacgtccatttccaagggaagatgagggacttcttcctgctggctgagccccaggagttctgggtggacaacagcacctcagtgtctgtccccatgctgtctggcgtgggcaccttccagcactggagcgacgcccaggacaacttctcagtgactcaagtgccctttactgagagcgcctgcttgctgctgattcagcctcactacgcctctgacctggacaaggtggagggtctcactttccagcaaaactccctcaactggatgaagaaactgtctccccgggccatccacctgaccatgccccgactggtgctgcgaggatcttatgacctgcaggacctgcttgcccaggctgagctgcccgccattctgggcaccgagctgaacctgcaaaaattgagcaatgacaacctcagggtggggaaggtgctgaacagcattctttttgaactcgaagcggatgagagagagcccacagagtctacccgacagctgaacaggcctgagttcttggaggtgaccctggaccgcccattcctgtttgctgtgtatgatcaaagtgccactgccctgcacttcctgggccgtgtggccaacccgctgagcccagcatgaggccagggccccagaacacagcgcctggcaaggcctctgcccctggcctttgaggcgaaggccagcagcagatatgtaactctggaaaaaccagcgatttgtcacccccagtctcccaccttttcttctaatgagtcaacttcgagctggaaagcagtcgtttctccttggtctaagtggtgctgcgtgagcagtaagaaacctgtggcagcacaaatgcgcctcccaggttgctgggtttattttagagaatgggggtggggaggcaagaaccagtgtttagcgcgggaccaccgttccaaaaagaattccaaccgaccagcttgtttgtgaaaca

SEQ ID NO: 4 - DNA, 1834 bp, Macaca fascicularis
  source 1..1834: mol_type = genomic DNA
tgtttcacaaacaagctggtcggttggaattctttttggaacggtggtcccgcgctaaacactggttcttgcctccccacccccattctctaaaataaacccagcaacctgggaggcgcatttgtgctgccacaggtttcttactgctcacgcagcaccacttagaccaaggagaaacgactgctttccagctcgaagttgactcattagaagaaaaggtgggagactgggggtgacaaatcgctggtttttccagagttacatatctgctgctggccttcgcctcaaaggccaggggcagaggccttgccaggcgctgtgttctggggccctggcctcatgctgggctcagcgggttggccacacggcccaggaagtgcagggcagtggcactttgatcatacacagcaaacaggaatgggcggtccagggtcacctccaagaactcaggcctgttcagctgtcgggtagactctgtgggctctctctcatccgcttcgagttcaaaaagaatgctgttcagcaccttccccaccctgaggttgtcattgctcaatttttgcaggttcagctcggtgcccagaatggcgggcagctcagcctgggcaagcaggtcctgcaggtcataagatcctcgcagcaccagtcggggcatggtcaggtggatggcccggggagacagtttcttcatccagttgagggagttttgctggaaagtgagaccctccaccttgtccaggtcagaggcgtagtgaggctgaatcagcagcaagcaggcgctctcagtaaagggcacttgagtcactgagaagttgtcctgggcgtcgctccagtgctggaaggtgcccacgccagacagcatggggacagacactgaggtgctgttgtccacccagaactcctggggctcagccagcaggaagaagtccctcatcttcccttggaaatggacgtaggtgttgaaaaccagggtgctgtccgcactggctcccgtcagggggctgctaatcttccatcctgtcacagcctgcatgaacctgtcaatcttctcagcagcgacttccaggtctgtgaagtccagagagcgtgggaggaccacaggggcatagagagccaggccctgcacaaacggctgcttcaggtgcagatctggggctgtgaagagacccaccactgtggacaacagcagctgggactggccgtcagccctgccctgggccaccagcaggccctgtacagcctgcagggcagagaggaccttgtgcgcatccagccgggaggtgcagttcttgtccttccaagggacgcccaggattgcctgtagcctgtcggctgtgtggtccaacgctcccaggtagagagaggccagggtgccaaagacagccgttggggagaggatggtggccccatggaccacgccccatagctcactgtgcatgccatatatacggaagcccaagaagttggccagcatcccgaccatcgcggctctcaacttgtcctcggtgtcgagttttgcggcaaccagcactagctggtcctgcagggccttttcatccacaggagacgtcttggcctgtatcggaacaggtgtgaaggtgggatctttgggcttcccagcatcggcctttgccagctgctcacaggtactctcattgtggatgacgaggtggaaggggtgtatgtacacccggtcacctgtggccaggccagcccaggccaggaggcagaggatggtggccctcaggctcacgctggcaggagccatctcggactggggtgctcgcttctgcatacccttctgctgtagtacccagaacaatggcagcttctt

SEQ ID NO: 5 - DNA, 1856 bp, Mus musculus
  source 1..1856: mol_type = genomic DNA
gatagctgtgcttgtctaggttggcgctgaaggatacacagaagcaaatgcacagatcggagatgactcccacgggggcaggcctgaaggccaccatcttctgcatcttgacctgggtcagcctgacggctggggaccgcgtatacatccaccccttccatctcctttaccacaacaagagcacctgcgcccagctggagaaccccagtgtggagacactcccagagtcaacgttcgagcctgtgcccattcaggccaagacctcccctgtgaatgagaagaccctgcatgatcagctcgtgctggccgccgagaagctagaggatgaggaccggaagcgggctgcccaggtcgcaatgatcgccaacttcgtgggcttccgcatgtacaagatgctgaatgaggcaggaagtggggccagtggggccatcctctcaccaccagctctctttggcaccctggtctctttctaccttggatccttagatcccacggccagccagctgcagacgctgctggatgtccctgtgaaggagggagactgcacctcccgactagatggacacaaggtcctcgctgccctgcgggccattcagggcttgctggtcacccagggtgggagcagcagccagacacccctgctacagtccattgtggtggggctcttcactgctccaggctttcgtctaaagcactcatttgttcagagcctggctctctttacccctgccctcttcccacgctctctggatttatccactgacccagttcttgccactgagaaaatcaacaggttcataaaggctgtgacagggtggaagatgaacttgccactggagggggtcagtacagacagcaccctacttttcaacacctacgttcacttccaaggaacgatgagaggtttctctcagctgcctggagtccatgaattctgggtggacaacagcatctcggtgtctgtgcccatgatctccggcactggcaacttccagcactggagtgacacccagaacaacttctccgtgacgtgcgtgcccctaggtgagagagccaccctgctgctcatccagccccactgcacctcagatctcgacagggtggaggccctcatcttccggaacgacctcctgacttggatagagaacccgcctcctcgggccatccgcctgactctgccccagctggaaatccgaggatcctacaatctgcaggacctgctggctgaggacaagctgcccacccttttgggtgcggaggcaaatctgaacaacattggtgacaccaacccccgagtgggagaggttctcaatagcatcctcctcgaactcaaagcaggagaggaggaacagccgaccacgtctgtccagcagcctggctcaccggaggcactggatgtgaccctgagcagccccttcctgttcgccatctacgagcaggactcaggcacgctgcactttctgggcagagtgaataacccccagagtgtggtgtgaggccttgtgcctagccatggagacaaggccggtgtcggagaaccgttctgggcaaaactcagtgctgtcacccctggctccccatcacgccttgtagcgcggcagaggccgtctccttggagactgcgctgaccgagaataaatgatgagcagcagagcctcctgggatgtgggtttgtttggatactggggtgacagccagaagctggcactctgcacaggactgccactctggaagaaatttggaccaaaaaactgtttgtgacaccaaaaagcacccccccttttttttatttgaggacagaaattgggttttaacattaaaatgcacattatccccttaaaaaaaaaaaaaaaaaa

SEQ ID NO: 6 - DNA, 1856 bp, Mus musculus
  source 1..1856: mol_type = genomic DNA
ttttttttttttttttttaaggggataatgtgcattttaatgttaaaacccaatttctgtcctcaaataaaaaaaagggggggtgctttttggtgtcacaaacagttttttggtccaaatttcttccagagtggcagtcctgtgcagagtgccagcttctggctgtcaccccagtatccaaacaaacccacatcccaggaggctctgctgctcatcatttattctcggtcagcgcagtctccaaggagacggcctctgccgcgctacaaggcgtgatggggagccaggggtgacagcactgagttttgcccagaacggttctccgacaccggccttgtctccatggctaggcacaaggcctcacaccacactctgggggttattcactctgcccagaaagtgcagcgtgcctgagtcctgctcgtagatggcgaacaggaaggggctgctcagggtcacatccagtgcctccggtgagccaggctgctggacagacgtggtcggctgttcctcctctcctgctttgagttcgaggaggatgctattgagaacctctcccactcgggggttggtgtcaccaatgttgttcagatttgcctccgcacccaaaagggtgggcagcttgtcctcagccagcaggtcctgcagattgtaggatcctcggatttccagctggggcagagtcaggcggatggcccgaggaggcgggttctctatccaagtcaggaggtcgttccggaagatgagggcctccaccctgtcgagatctgaggtgcagtggggctggatgagcagcagggtggctctctcacctaggggcacgcacgtcacggagaagttgttctgggtgtcactccagtgctggaagttgccagtgccggagatcatgggcacagacaccgagatgctgttgtccacccagaattcatggactccaggcagctgagagaaacctctcatcgttccttggaagtgaacgtaggtgttgaaaagtagggtgctgtctgtactgaccccctccagtggcaagttcatcttccaccctgtcacagcctttatgaacctgttgattttctcagtggcaagaactgggtcagtggataaatccagagagcgtgggaagagggcaggggtaaagagagccaggctctgaacaaatgagtgctttagacgaaagcctggagcagtgaagagccccaccacaatggactgtagcaggggtgtctggctgctgctcccaccctgggtgaccagcaagccctgaatggcccgcagggcagcgaggaccttgtgtccatctagtcgggaggtgcagtctccctccttcacagggacatccagcagcgtctgcagctggctggccgtgggatctaaggatccaaggtagaaagagaccagggtgccaaagagagctggtggtgagaggatggccccactggccccacttcctgcctcattcagcatcttgtacatgcggaagcccacgaagttggcgatcattgcgacctgggcagcccgcttccggtcctcatcctctagcttctcggcggccagcacgagctgatcatgcagggtcttctcattcacaggggaggtcttggcctgaatgggcacaggctcgaacgttgactctgggagtgtctccacactggggttctccagctgggcgcaggtgctcttgttgtggtaaaggagatggaaggggtggatgtatacgcggtccccagccgtcaggctgacccaggtcaagatgcagaagatggtggccttcaggcctgcccccgtgggagtcatctccgatctgtgcatttgcttctgtgtatccttcagcgccaacctagacaagcacagctatc

SEQ ID NO: 7 - DNA, 1958 bp, Rattus norvegicus
  source 1..1958: mol_type = genomic DNA
ccttgctccatcttggctaagcctggattcccatggtcccccgacctgggtcctcccccagcctctgtacagagtagcctgggaatagatccatcttcaccccctcgagtataaataaggctgcttggttcaccaggggatagctgtgcttgtctgggctggagctaaaggacacacagaagcaagtccacagatccgtgatgactcccacgggggcaggcctgaaggccaccatcttctgcatcctgacctgggtcagcctgacagctggggaccgcgtatacatccacccctttcatctcctctactacagcaagagcacctgcgcccagctggagaaccccagtgtggagacgctcccagagccaacctttgagcctgtgcccattcaggccaagacctcccccgtggatgagaagaccctgcgagataagctcgtgctggccactgagaagctagaggctgaggatcggcagcgagctgcccaggtcgcgatgattgccaacttcatgggtttccgcatgtacaagatgctgagtgaggcaagaggtgtagccagtggggccgtcctctctccaccggccctctttggcaccctggtctctttctaccttggatcgttggatcccacggccagccagttgcaggtgctgctgggcgtccctgtgaaggagggagactgcacctcccggctggacggacataaggtcctcactgccctgcaggctgttcagggcttgctggtcacccagggtggaagcagcagccagacacccctgctacagtccaccgtggtgggcctcttcactgccccaggcttgcgcctaaaacagccatttgttgagagcttgggtcccttcacccccgccatcttccctcgctctctggacttatccactgacccagttcttgctgcccagaaaatcaacaggtttgtgcaggctgtgacagggtggaagatgaacttgccactagagggggtcagcacggacagcaccctatttttcaacacctacgttcacttccaagggaagatgagaggcttctcccagctgactgggctccatgagttctgggtggacaacagcacctcagtgtctgtgcccatgctctcgggcactggcaacttccagcactggagtgacgcccagaacaacttctccgtgacacgcgtgcccctgggtgagagtgtcaccctgctgctgatccagccccagtgcgcctcagatctcgacagggtggaggtcctcgtcttccagcacgacttcctgacttggataaagaacccgcctcctcgggccatccgtctgaccctgccgcagctggaaattcggggatcctacaacctgcaggacctgctggctcaggccaagctgtctacccttttgggtgctgaggcaaatctgggcaagatgggtgacaccaacccccgagtgggagaggttctcaacagcatcctccttgaactccaagcaggcgaggaggagcagcccacagagtctgcccagcagcctggctcacccgaggtgctggacgtgaccctgagcagtccgttcctgttcgccatctacgagcgggactcaggtgcgctgcactttctgggcagagtggataacccccaaaatgtggtgtgatgcctcctgtgtagccatggagacaaggccagcgtcagagagctatcctgggcaaaaatcagtgccttcacccctggcttcccgtcactccttccagcaaggcagaggccgtctccttggagatggcgctaactgagaataaatgatgagcagcagcctcctggggtgtgggtttgtttggacactggggtgagagccaggagctggcactctgtataggaggactgccatcctggaaaaaaaaaatggaccaaacaactgtttgtgaaataaaaaaaaaaaaattccctttttatttgaaaaaaaaaaaaaaaaaaaaaaaaa

SEQ ID NO: 8 - DNA, 1958 bp, Rattus norvegicus
  source 1..1958: mol_type = genomic DNA
tttttttttttttttttttttttttcaaataaaaagggaatttttttttttttatttcacaaacagttgtttggtccattttttttttccaggatggcagtcctcctatacagagtgccagctcctggctctcaccccagtgtccaaacaaacccacaccccaggaggctgctgctcatcatttattctcagttagcgccatctccaaggagacggcctctgccttgctggaaggagtgacgggaagccaggggtgaaggcactgatttttgcccaggatagctctctgacgctggccttgtctccatggctacacaggaggcatcacaccacattttgggggttatccactctgcccagaaagtgcagcgcacctgagtcccgctcgtagatggcgaacaggaacggactgctcagggtcacgtccagcacctcgggtgagccaggctgctgggcagactctgtgggctgctcctcctcgcctgcttggagttcaaggaggatgctgttgagaacctctcccactcgggggttggtgtcacccatcttgcccagatttgcctcagcacccaaaagggtagacagcttggcctgagccagcaggtcctgcaggttgtaggatccccgaatttccagctgcggcagggtcagacggatggcccgaggaggcgggttctttatccaagtcaggaagtcgtgctggaagacgaggacctccaccctgtcgagatctgaggcgcactggggctggatcagcagcagggtgacactctcacccaggggcacgcgtgtcacggagaagttgttctgggcgtcactccagtgctggaagttgccagtgcccgagagcatgggcacagacactgaggtgctgttgtccacccagaactcatggagcccagtcagctgggagaagcctctcatcttcccttggaagtgaacgtaggtgttgaaaaatagggtgctgtccgtgctgaccccctctagtggcaagttcatcttccaccctgtcacagcctgcacaaacctgttgattttctgggcagcaagaactgggtcagtggataagtccagagagcgagggaagatggcgggggtgaagggacccaagctctcaacaaatggctgttttaggcgcaagcctggggcagtgaagaggcccaccacggtggactgtagcaggggtgtctggctgctgcttccaccctgggtgaccagcaagccctgaacagcctgcagggcagtgaggaccttatgtccgtccagccgggaggtgcagtctccctccttcacagggacgcccagcagcacctgcaactggctggccgtgggatccaacgatccaaggtagaaagagaccagggtgccaaagagggccggtggagagaggacggccccactggctacacctcttgcctcactcagcatcttgtacatgcggaaacccatgaagttggcaatcatcgcgacctgggcagctcgctgccgatcctcagcctctagcttctcagtggccagcacgagcttatctcgcagggtcttctcatccacgggggaggtcttggcctgaatgggcacaggctcaaaggttggctctgggagcgtctccacactggggttctccagctgggcgcaggtgctcttgctgtagtagaggagatgaaaggggtggatgtatacgcggtccccagctgtcaggctgacccaggtcaggatgcagaagatggtggccttcaggcctgcccccgtgggagtcatcacggatctgtggacttgcttctgtgtgtcctttagctccagcccagacaagcacagctatcccctggtgaaccaagcagccttatttatactcgagggggtgaagatggatctattcccaggctactctgtacagaggctgggggaggacccaggtcgggggaccatgggaatccaggcttagccaagatggagcaagg

SEQ ID NO: 9 - RNA, 23 nt, synthetic construct
  source 1..23: mol_type = other RNA
tgtactctcattgtggatgacga

SEQ ID NO: 10 - RNA, 21 nt, synthetic construct
  source 1..21: mol_type = other RNA
gtcatccacaatgagagtaca

SEQ ID NO: 11 - RNA, 23 nt, synthetic construct
  source 1..23: mol_type = other RNA
  modified_base 1: mod_base = OTHER; note = 2'-O-methyluridine-3'-phosphorothioate
  modified_base 2: mod_base = OTHER; note = 2'-deoxy-2'-fluoroguanosine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = Thymidine-glycol nucleic acid (GNA), S-Isomer
  modified_base 7: mod_base = OTHER; note = 2'-O-methylcytidine-3'-phosphate
  modified_base 8: mod_base = OTHER; note = 2'-O-methyluridine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methylcytidine-3'-phosphorothioate
  modified_base 22: mod_base = OTHER; note = 2'-O-methylguanosine-3'-phosphorothioate
  modified_base 23: mod_base = OTHER; note = 2'-O-methyladenosine-3'-phosphate
tgtactctcattgtggatgacga

SEQ ID NO: 12 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methyluridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methylguanosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methylcytidine-3'-phosphate
  modified_base 21: mod_base = OTHER; note = 2'-O-methyladenosine
gtcatccacaatgagagtaca